(19) United States
(12) Patent Application Publication
(10) Pub. No.: US 2025/0066320 A1
(43) Pub. Date: Feb. 27, 2025

(54) Active Ingredients with Sialagogue and Tingling/Fizzy Effect, and Preparations Containing Same

(71) Applicant: Symrise AG, Holzminden (DE)

(72) Inventors: Fabia HENTSCHEL, Holzminden (DE); Bernd WIEDWALD, Holzminden (DE); Susanne PAETZ, Höxter (DE); Ulrike SIMCHEN, Holzminden (DE); Michael BACKES, Holzminden (DE); Judith MINAKAR, Holzminden (DE)

(21) Appl. No.: 18/722,561

(22) PCT Filed: Dec. 23, 2021
(86) PCT No.: PCT/EP2021/087586

§ 371 Date: Jun. 20, 2024

(51) Int. Cl. C07D 401/06; A23L 27/00; A23L 27/20; A61K 31/445; A61K 31/496; A61K 31/55; C07D 211/16; C07D 243/08; C07D 295/192

(52) U.S. Cl. CPC C07D 401/06; A23L 27/2054; A23L 27/88; A61K 31/445; A61K 31/496; A61K 31/55; C07D 211/16; C07D 243/08; C07D 295/192

(57) ABSTRACT

The present invention is in the field of flavoring agents and relates to new representatives of this group, in particular active ingredients with a mouth-watering effect, the use of these active ingredients and preparations comprising them, preferably preparations for nutrition, enjoyment or oral hygiene or oral pharmaceutical preparations.

### FIELD OF THE INVENTION

The present invention is in the field of flavoring agents and relates to new representatives of this group, in particular active ingredients with a mouth-watering and tingling/fizzing effect, the use of these active ingredients and preparations comprising them, preferably preparations for nutrition, enjoyment or oral hygiene or oral pharmaceutical preparations.

### TECHNOLOGICAL BACKGROUND

The overall organoleptic impression of a product usually consists of aroma, taste, trigeminal perception and texture. The volatile compounds of the aroma are perceived by the olfactory receptors of the nose. The volatile compounds can be absorbed via the olfactory mucosa of the nose or retronasally, i.e., backwards via the palate. In contrast to aroma, the taste of a product is perceived gustatory via the human taste organ. Taste refers to the qualities of sweet, sour, salty, bitter and umami, which are perceived on the tongue with the taste buds. The trigeminal sensations of a product such as mouth-watering, tingling/fizzing, numbing, warming, burning, spicy, astringent, pungent, stinging, cooling, etc. are caused by substances or active ingredients that stimulate the facial nerve nervus trigeminus. The stimulation takes place in the free sensitive end area of the trigeminal neuron. Free nerve endings that react to chemical stimuli are found in the eye and in the mucous membranes of the nasal and oral cavities.

Mouth-watering substances or active ingredients that stimulate the flow of saliva in the mouth have long played an important role in food, luxury food, oral hygiene and pharmaceutical preparations. Saliva-stimulating active ingredients are used, for example, to stimulate appetite, to combat pathological dryness of the mouth or to improve oral hygiene by flushing harmful substances or germs out of the oral cavity through increased saliva flow.

### STATE OF THE ART

Natural or synthetic substances or active ingredients with a mouth-watering and/or tingling/fizzing effect are known from the state of the art:

In food preparations, edible acids such as citric acid, tartaric acid or malic acid are usually used to stimulate salivation. Special substances that stimulate the salivary gland, such as pilocarpine extracted from the jaborandi tree, are used to treat dry mouth. However, such highly potent cholinergic parasympathomimetics usually have serious side effects and are sometimes toxic. The naturally occurring alkamides spilanthol (e.g., spilanthol from jambu extract, also known as paracress) and pellitorin have a saliva-stimulating and tingling effect, but also have a long-lasting and anaesthetic effect in the mouth, which is not desirable for all applications. They are also difficult to synthesize due to their highly unsaturated structure.

### TASK OF THE INVENTION

The primary task of the present invention was to identify new substances or active ingredients which produce a trigeminal effect when consumed, preferably active ingredients for stimulating the flow of saliva in the mouth and for producing a sensation of tingling in the mouth, but at the same time have significantly fewer off-notes (aftertaste) and/or off-flavors, such as a bitter taste.

In addition, these substances or active ingredients should produce a rapid and immediate onset and long-lasting trigeminal effect even at low concentrations. Furthermore, these substances or active ingredients should have a relatively neutral odor and taste profile so that they can be incorporated into preparations, in particular dietary preparations, oral hygiene preparations or oral pharmaceutical preparations. The specified active ingredients should ultimately be easily accessible and stable synthetically.

To solve the problem according to the invention, the search was primarily for active ingredients that can impart a particularly rapid and immediate onset and long-lasting trigeminal effect as described above. The active ingredients should be efficient, i.e., they should have a mouth-watering effect or sensation even in low concentrations.

A further task was to provide substances or active ingredients which, in addition to the effects described above, also reduce or mask off-notes or off-flavors. This relates in particular to bitter, astringent, dry, dusty, mealy, chalky or metallic, preferably bitter or astringent, off-notes or off-flavors.

The problem is solved by the objects of the independent patent claims.

Further aspects of the present invention are apparent from the wording of the dependent claims, the following description and the examples.

### DETAILED DESCRIPTION OF THE INVENTION

The primary task of the present invention is solved according to the invention by an active ingredient of the general formula (I)

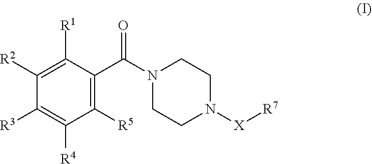

or the general formula (II)

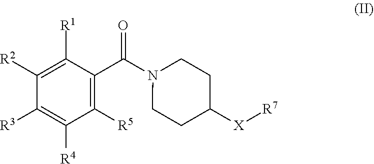

or the general formula (III)

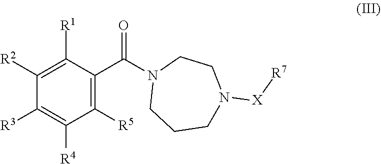

in which the radicals R1 to R5 and R7 can each be identical or different and independently of one another have the following meanings:
R1 Hor
   OH; or
   Q; or
   an optionally substituted alkyl group; or
   an optionally substituted alkenyl group; or
   an optionally substituted alkoxy group; or
   an optionally substituted alkylthio group; or
   an optionally substituted acyl group R—(C═O)—; or
   an optionally substituted cycloalkyl group; or
   an optionally substituted aryl group; or
   an optionally substituted heterocyclyl group; or
   is an optionally substituted heteroaryl group; or
   R1 forms with the adjacent radical R2, together with the carbon atoms to which they are attached, an optionally substituted four-, five-, six- or seven-membered cycloalkyl or aryl ring, or forms an optionally substituted four-, five-, six- or seven-membered heterocyclyl or heteroaryl ring, wherein the heterocyclyl or heteroaryl ring may comprise one, two or more identical or different heteroatoms selected from the group consisting of nitrogen, oxygen and sulfur;
R2 Hor
   OH; or
   Q; or
   an optionally substituted alkyl group; or
   an optionally substituted alkenyl group; or
   an optionally substituted alkoxy group; or
   an optionally substituted alkylthio group; or
   an optionally substituted acyl group R—(C═O)—; or
   an optionally substituted cycloalkyl group; or
   an optionally substituted aryl group; or
   an optionally substituted heterocyclyl group; or
   is an optionally substituted heteroaryl group; or
   R2 forms with one of the adjacent radicals R1 or R3, together with the carbon atoms to which they are attached, an optionally substituted four-, five-, six- or seven-membered cycloalkyl or aryl ring, or forms an optionally substituted four-, five-, six- or seven-membered heterocyclyl or heteroaryl ring, wherein the heterocyclyl or heteroaryl ring may comprise one, two or more identical or different heteroatoms selected from the group consisting of nitrogen, oxygen and sulfur; and/or
R3 Hor
   OH; or
   Q; or
   an optionally substituted alkyl group; or
   an optionally substituted alkenyl group; or
   an optionally substituted alkoxy group; or
   an optionally substituted alkylthio group; or
   an optionally substituted acyl group R—(C═O)—; or
   an optionally substituted cycloalkyl group; or
   an optionally substituted aryl group; or
   an optionally substituted heterocyclyl group; or
   is an optionally substituted heteroaryl group; or
   R3 forms with one of the adjacent radical R2 or R4, together with the carbon atoms to which they are attached, an optionally substituted four-, five-, six- or seven-membered cycloalkyl or aryl ring, or forms an optionally substituted four-, five-, six- or seven-membered heterocyclyl or heteroaryl ring, wherein the heterocyclyl or heteroaryl ring may comprise one, two or more identical or different heteroatoms selected from the group consisting of nitrogen, oxygen and sulfur;
R4 Hor
   OH; or
   Q; or
   an optionally substituted alkyl group; or
   an optionally substituted alkenyl group; or
   an optionally substituted alkoxy group; or
   an optionally substituted alkylthio group; or
   an optionally substituted acyl group R—(C═O)—; or
   an optionally substituted cycloalkyl group; or
   an optionally substituted aryl group; or
   an optionally substituted heterocyclyl group; or
   is an optionally substituted heteroaryl group; or
   R4 forms with one of the adjacent radical R3 or R5, together with the carbon atoms to which they are attached, an optionally substituted four-, five-, six- or seven-membered cycloalkyl or aryl ring, or forms an optionally substituted four-, five-, six- or seven-membered heterocyclyl or heteroaryl ring, wherein the heterocyclyl or heteroaryl ring may comprise one, two or more identical or different heteroatoms selected from the group consisting of nitrogen, oxygen and sulfur; and/or
R5 Hor
   OH; or
   Q; or
   an optionally substituted alkyl group; or
   an optionally substituted alkenyl group; or
   an optionally substituted alkoxy group; or
   an optionally substituted alkylthio group; or
   an optionally substituted acyl group R—(C═O)—; or
   an optionally substituted cycloalkyl group; or
   an optionally substituted aryl group; or
   an optionally substituted heterocyclyl group; or
   is an optionally substituted heteroaryl group; or
   R5 forms with the adjacent radical R4, together with the carbon atoms to which they are attached, an optionally substituted four-, five-, six- or seven-membered cycloalkyl or aryl ring or forms an optionally substituted four-, five-, six- or seven-membered heterocyclyl or heteroaryl ring, wherein the heterocyclyl or heteroaryl ring forms one, two or more identical or different heterocyclic or heteroaryl rings, six- or seven-membered heterocyclyl or heteroaryl ring, wherein the heterocyclyl or heteroaryl ring may comprise one, two or more identical or different heteroatoms selected from the group consisting of nitrogen, oxygen and sulfur; and/or
R7 an optionally substituted cycloalkyl group; or
   an optionally substituted aryl group; or
   an optionally substituted heterocyclyl group; or
   is an optionally substituted heteroaryl group;
X an optionally substituted alkyl group; or
   an optionally substituted alkenyl group; or
   an optionally substituted alkoxy group; or
   an optionally substituted alkylthio group; or
   is an optionally substituted acyl group R(C═O)—;
optionally one or both nitrogen atom(s) of the saturated six-membered ring in the general formula (I) or the nitrogen atom of the saturated six-membered ring in the general formula (II) or one or both nitrogen atom(s) of the saturated seven-membered ring in the general formula (III) is/are replaced by a C atom;
optionally the saturated six-membered ring in the general formula (I) or the general formula (II) or the saturated seven-membered ring in the general formula (III) is mono- or polysubstituted;
wherein
the group Q is a radical selected from the group consisting of: halogen, —OY, —SY, —NZZ, —NY, —N—OY, trihalomethyl, —CF₃, —CN, —OCN, —SCN, —NO, —NO₂, —NH₂, —N₃, —S(O)₂Y, —S(O)₂OY, —OS(O)₂Y, —OS(O)₂OY, —P(O)(OY)₂, —P(O)(OY)(OY), —C(O)Y, —C(S)Y, —C(NY)Y, —C(O)OY, —C(S)OY, —C(O)NZZ, —C(NY)NZZ, —OC(O)Y, —OC(S)Y, —OC(O)OY, —OC(S)OY, —NYC(O)Y, —NYC(S)Y, —NYC(O)OY, —NYC(S)OY, —NYC(O)NZZ, —NYC(NY)Y or —NYC(NY)NZZ;
wherein
Y is selected from the group consisting of:
   Hydrogen;
   alkyl group, in particular C₁- to C₁₀-alkyl group, in particular C₁- to C₆-alkyl group, especially C₁-, C₂-, C₃- or C₄-alkyl group;
   alkoxy group, in particular C₁-C₆-alkoxy group, in particular optionally substituted C₁-, C₂-, C₃- or C₄-alkoxy group;
   alkylthio group, in particular C₁- to C₆-alkylthio group, in particular optionally substituted C₁-, C₂-, C₃- or C₄-alkylthio group;
   Cycloalkyl group;
   Aryl Group;
   Heterocyclyl group;
   heteroaryl group; and/or
Z represents Y or alternatively, two Z's together with the nitrogen atom to which they are attached represent a four, five, six or seven membered heterocyclyl ring or heteroaryl ring, wherein the heterocyclyl ring or heteroaryl ring may comprise one, two or more identical or different heteroatoms selected from the group consisting of nitrogen, oxygen and sulfur;
the optionally substituted alkyl group is an optionally substituted C₁- to C₁₀-alkyl group, in particular an optionally substituted C₁- to C₆-alkyl group, in particular an optionally substituted C₁-, C₂-, C₃- or C₄-alkyl group;
the optionally substituted alkenyl group is an optionally substituted C₁- to C₁₀-alkenyl group, in particular an optionally substituted C₁- to C₆-alkenyl group, is an optionally substituted C₁-, C₂-, C₃- or C₄-alkenyl group;
the optionally substituted alkoxy group is an optionally substituted C₁- to C₁₀-alkoxy group, in particular an optionally substituted C₁- to C₆-alkoxy group, is an optionally substituted C₁-, C₂-, C₃- or C₄-alkoxy group;
the optionally substituted alkylthio group is an optionally substituted C₁- to C₁₀-alkylthio group, in particular an optionally substituted C₁- to C₆-alkylthio group, is in particular an optionally substituted C₁-, C₂-, C₃- or C₄-alkylthio group;
in the acyl group R—(C═O)— the radical R represents hydrogen or an optionally substituted C₁- to C₁₀-alkyl group, in particular an optionally substituted C₁- to C₆-alkyl group,
in particular represents an optionally substituted C₁-, C₂-, C₃- or C₄-acyl group;
the optionally substituted cycloalkyl group is an optionally substituted four- to ten-membered, in particular five-, six- or seven-membered, monocyclic cycloalkyl group or an optionally substituted nine- to twelve-membered polycyclic, in particular bicyclic, cycloalkyl group;
the optionally substituted aryl group is an optionally substituted four- to ten-membered, in particular five-, six- or seven-membered, monocyclic aryl group or an optionally substituted nine- to twelve-membered polycyclic, in particular bicyclic aryl group;
the optionally substituted heterocyclyl group is an optionally substituted four-, five-, six- or seven-membered monocyclic heterocyclyl group or an optionally substituted nine- to twelve-membered polycyclic, in particular bicyclic, heterocyclyl group, wherein the heterocyclyl group may comprise one, two, three or four identical or different heteroatoms selected from the group consisting of nitrogen, oxygen and sulfur;
the optionally substituted heteroaryl group is an optionally substituted four-, five, six- or seven-membered monocyclic heteroaryl group or an optionally substituted nine- to twelve-membered polycyclic, in particular bicyclic, heteroaryl group, wherein the heteroaryl group may comprise one, two, three or four identical or different heteroatoms selected from the group consisting of nitrogen, oxygen and sulfur;
   and their salts, in particular acid addition salts, with inorganic or organic acids.

The active compounds according to the general formula (I), (II) or (III) of the invention may be present in stereoisomerically pure form or as mixtures of different stereoisomers.

According to the general formula (I) as reproduced above, the structural unit N—X—R⁷ is located in the para position to the nitrogen atom, or optionally to the carbon atom if the nitrogen atom is replaced by a carbon atom, of the six-membered ring in the general formula (I). In an alternative variant according to the first aspect of the present invention, the structural unit N—X—R⁷ in the formula (I) can also be arranged at a different position of the six-membered heterocyclic ring in the general formula (I), namely in the ortho or meta position to the nitrogen atom or optionally to the carbon atom if the nitrogen atom is replaced by a carbon atom.

According to the general formula (II) as reproduced above, the structural unit C—X—R⁷ is located in the para position to the nitrogen atom, or optionally to the carbon atom if the nitrogen atom is replaced by a carbon atom, of the six-membered ring in the general formula (II). In an alternative variant according to the first aspect of the present invention, the structural unit C—X—R⁷ in the formula (II) can also be arranged at a different position of the six-membered heterocyclic ring in the general formula (II), namely in the ortho or meta position to the nitrogen atom or optionally to the carbon atom if the nitrogen atom is replaced by a carbon atom.

According to the general formula (III) as reproduced above, the structural unit N—X—R⁷ is located in the para position to the nitrogen atom, or optionally to the carbon atom if the nitrogen atom is replaced by a carbon atom, of the seven-membered ring in the general formula (III). In an alternative variant according to the first aspect of the present invention, the structural unit N—X—R⁷ in the formula (III) can also be arranged at a different position of the seven-membered heterocyclic ring in the general formula (III), namely in the ortho or meta position to the nitrogen atom or optionally to the carbon atom if the nitrogen atom is replaced by a carbon atom.

In the context of the present invention and in particular for the definition of the active ingredients of the general formulae (I), (II) and (III), the following general meanings apply:

The term “or” or “and/or” is used as a function word to indicate that two words or expressions should be taken together or separately.

The terms “comprising”, “with”, “including” and “containing” are to be understood as open terms, i.e., “comprising, including or containing”, but not limited to.

The end points of all areas that are directed to the same component or property can be combined inclusively and independently of each other.

The term “active compound(s)” or “active compound(s) of the present invention” refers to all compounds encompassed by the structural formulae formula (I), formula (II) or formula (II) disclosed herein, and includes any subgenus and specific compounds within the formula whose structure is disclosed herein. The active ingredients can be identified by either their chemical structure and/or their chemical name. When the chemical structure and chemical name are in conflict, the chemical structure determines the identity of the compound. The compounds described herein may contain one or more chiral centers and/or double bonds and therefore may exist as stereoisomers, such as double bond isomers, i.e., geometric isomers, enantiomers, or diastereomers. Accordingly, the chemical structures of the general formulae (I) to (III) shown here comprise all possible enantiomers and diastereomers or stereoisomers.

In the context of the present invention, an active ingredient is an active substance, for example in a preparation, as opposed to an excipient. In other words, an active ingredient is a substance or component that has a specific effect or causes a specific reaction. In the context of the present invention, the active ingredients according to the invention are substances that have a trigeminal effect. Preferably, the active ingredients according to the present invention elicit a salivary and/or tingling/fizzing effect in the mouth.

The term “mouth-watering or tingling/fizzing effect” in the context of the present invention means that when an active ingredient according to the invention is consumed or applied, the flow of saliva in the mouth is stimulated by stimulating the trigeminal nerve and/or a tingling/fizzing sensation is triggered in the mouth.

The terms “alkyl” or “alkyl group” or “alkyl radical”, used synonymously, by themselves or as part of another substituent according to the present invention, refer to a saturated linear or branched monovalent hydrocarbon radical obtained by removing a hydrogen atom from a single carbon atom of a starting alkane.

In a preferred variant, the term “alkyl group” comprises saturated linear or branched hydrocarbon radicals having 1 to 10, 1 to 8, 1 to 6 or 1 to 4 carbon atoms.

If the alkyl group is further alkylene residue or an alkylene group. In other words, the term “alkylene” also refers to a divalent alkyl. For example, —CH₂CH₃ is an ethyl, while —CH₂CH₂— is an ethylene.

The term “alkylene group” alone or as part of another substituent refers to a saturated linear or branched divalent hydrocarbon radical obtained by removing two hydrogen atoms from a single carbon atom or two different carbon atoms of a starting alkane.

According to the present invention, an alkyl group or an alkylene group comprises 1 to 10 carbon atoms. In other preferred variants, an alkyl group or alkylene group comprises 1 to 6 carbon atoms. Most preferred is an alkyl group or alkylene group with 1 to 4 carbon atoms.

Most preferably, according to the invention, the alkyl group or alkenyl group is a saturated linear or branched C₁- to C₄-alkyl group or a saturated linear or branched C₁- to C₄-alkylene group.

Preferred alkyl groups or alkyl radicals include, but are not limited to: C₁- to C₆-alkyl comprising methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1 2-trimethylpropyl, 1,2,2-trimethylpropyl, 1-ethyl-1-methylpropyl and 1-ethyl-2-methylpropyl.

The alkyl group or the alkylene group, as defined above, is optionally substituted.

The terms “alkenyl” or “alkenyl group” or “alkenyl radical”, used synonymously, by themselves or as part of another substituent according to the present invention, refer to an unsaturated linear or branched monovalent hydrocarbon radical having at least one carbon-carbon double bond. The radical may be in either the cis or trans conformation around the double bond(s).

In preferred variants according to the present invention, an alkenyl group comprises 1 to 10 carbon atoms. In other preferred variants, an alkenyl group comprises 1 to 6 carbon atoms. In still further preferred variants, an alkenyl group comprises 1 to 4 carbon atoms.

Most preferably, the alkenyl group is a mono- or di-unsaturated linear or branched C₁- to C₄-alkenyl group.

Preferred alkenyl groups or alkenyl radicals include, but are not limited to, ethenyl, propenyls such as prop-1-en-1-yl, prop-1-en-2-yl, prop-2-en-1-yl (allyl), prop-2-en-2-yl, cycloprop-1-en-1-yl, cycloprop-2-en-1-yl, butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl and the like.

The alkenyl group, as defined above, is optionally substituted.

The terms “alkoxy” or “alkoxy group” or “alkoxy radical”, used synonymously, by themselves or as part of another substituent according to the present invention, refer to a radical of formula —O—R, wherein R is alkyl or substituted alkyl, as defined above.

According to the present invention, an alkoxy group comprises 1 to 10 carbon atoms. In other preferred variants, an alkoxy group comprises 1 to 6 carbon atoms. An alkoxy group with 1 to 4 carbon atoms is most preferred.

Most preferably, the alkoxy group is a saturated linear or branched C₁- to C₄ alkoxy group.

Preferred alkoxy groups or alkoxy radicals include, but are not limited to: C₁ to C₆-alkoxy comprising C₁- to C₄-alkoxy, such as, for example methoxy, ethoxy, n-propoxy, 1-methylethoxy, butoxy, 1-methylpropoxy, 2-methylpropoxy or 1,1-dimethylethoxy; as well as pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

The alkoxy group, as defined above, is optionally substituted.

The terms “alkylthio” or “alkylthio group” or “alkylthio moiety”, used synonymously, by themselves or as part of another substituent according to the present invention, refer to a radical of the formula —S—R, wherein R is alkyl or substituted alkyl, as defined above.

According to the present invention, an alkylthio group comprises 1 to 10 carbon atoms. In other preferred variants, an alkylthio group comprises 1 to 6 carbon atoms.

Most preferred is an alkylthio group with 1 to 4 carbon atoms.

Most preferably, the alkylthio group is a saturated linear or branched C₁- to C₄-alkylthio group.

The alkylthio group, as defined above, is optionally substituted.

The terms “acyl” or “acyl group” or “acyl residue”, which are used synonymously, by themselves or as part of another substituent according to the present invention, refer to a radical —R(C═O)—, where R is hydrogen, alkyl, substituted alkyl, cycloalkyl, substituted cycloalkyl, aryl, substituted aryl, arylalkyl, substituted arylalkyl, heteroalkyl, substituted heteroalkyl, heteroarylalkyl or substituted heteroarylalkyl as defined herein.

According to the present invention, an acyl group comprises an alkyl group having 1 to 10 carbon atoms. In other preferred variants, an acyl group comprises an alkyl group of 1 to 6 carbon atoms. Most preferably, an acyl group comprises an alkyl group having 1 to 4 carbon atoms.

Representative examples include, but are not limited to, formyl, acetyl, propionyl, butyryl, valeryl, benzoyl, cyclohexylcarbonyl, cyclohexylmethylcarbonyl, benzylcarbonyl, and the like.

Most preferably, the acyl group is a linear or branched C₁- to C₄-acyl group.

The acyl group, as defined above, is optionally substituted.

The terms “cycloalkyl” or “cycloalkyl group” or “cycloalkyl radical”, used synonymously, by themselves or as part of another substituent according to the present invention, refer to a saturated non-aromatic cyclic monovalent hydrocarbon radical in which the carbon atoms are ring-linked, and which has no heteroatom.

The carbon ring can occur as a monocyclic compound, which has only a single ring, or as a polycyclic compound, which has two or more rings.

In a preferred variant, the term “cycloalkyl” comprises a four- to ten-membered monocyclic cycloalkyl group or a nine- to twelve-membered polycyclic, in particular bicyclic, cycloalkyl group. In other even more preferred variants, the cycloalkyl group comprises a five- to seven-membered monocyclic cycloalkyl group. Most preferred is a monocyclic C₅—, C₆- or C₇-cycloalkyl group.

Preferred cycloalkyl groups include, but are not limited to, saturated carbocyclic radicals having from 4 to 10 carbon atoms comprising cyclobutyl, cyclopentyl, cyclohexyl, cycloheptyl, cyclooctyl, cyclononyl, and cyclodecyl; cyclopentyl, cyclohexyl, and cycloheptyl are preferred.

According to the invention, the terms “cycloalkyl” or “cycloalkyl group” or “cycloalkyl radical”, which are used synonymously, also include cycloalkenyls, i.e., unsaturated cyclic hydrocarbon radicals containing C═C double bonds between two carbon atoms of the ring molecule. In a broader sense, cycloalkenyls are compounds with one, two or more double bond(s), whereby the number of possible, mostly conjugated double bonds in the molecule depends on the ring size.

Typical cycloalkenyls include, but are not limited to, cyclopropenyl, cyclopentenyl, cyclohexenyl, cyclopentadienyl and the like.

According to the invention, the term “cycloalkyl” or “cycloalkyl group” or “cycloalkyl radical” also includes cycloalkynyls, i.e., unsaturated cyclic hydrocarbon radicals containing C≡C triple bonds between two carbon atoms of the ring molecule, the triple bond being dependent on the ring size for reasons of ring tension.

Typical cycloalkynes include cyclooctyne.

The cycloalkyl group or the cycloalkyl residue can be attached to an atom of the molecule of formula (I), formula (II) or formula (III) via any suitable C atom.

The cycloalkyl group, as defined above, is optionally substituted.

The terms “aryl” or “aryl group” or “aryl radical”, used synonymously, by themselves or as part of another substituent according to the present invention, refer to a monovalent aromatic hydrocarbon radical derived by removing a hydrogen atom from a single carbon atom of an aromatic ring system.

The carbon ring can occur as a monocyclic compound, which has only a single ring, or as a polycyclic compound, which has two or more rings.

In a preferred variant, the term “aryl” comprises a four- to ten-membered monocyclic aryl group or a nine- to twelve-membered polycyclic, in particular bicyclic, aryl group. In other even more preferred variants, the aryl group comprises a five-, six- or seven-membered monocyclic aryl group. Most preferred according to the invention are monocyclic C₅—, C₆- or C₇-aryl groups.

Preferred aryl radicals include, but are not limited to, benzene, phenyl, biphenyl, naphthyl such as 1- or 2-naphthyl, tetrahydronaphthyl, fluorenyl, indenyl and phenanthrenyl. Typical carboaryl radicals further include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane. Indene, naphthalene, octacene, octaphene, octalene, ovalene, penta-2,4-diene, pentacene, pentalene, pentaphene, perylene, phenalene, phenanthrene, picene, pleiades, pyrene, pyranthrene, rubicene, triphenylene, trinaphthalene and the like.

Preferred aromatic polycyclic aryl radicals or aryl groups according to the invention include, but are not limited to, naphthalene, biphenyl, etc.

The aryl group or the aryl residue can be bonded to an atom of the molecule of formula (I), formula (II) or formula (III) via any suitable C atom of the aryl group.

The aryl group, as defined above, is optionally substituted.

The terms “heterocyclyl” or “heterocyclyl group” or “heterocyclyl residue”, used synonymously, by themselves or as part of other substituents, refer to alkyl groups in which one or more of the carbon atom(s) have been independently replaced by the same or a different heteroatom. The heteroatoms or heteroatomic groups may be located at any internal position of the alkyl group.

The term “heterocyclyl” alone or as part of another substituent according to the present invention refers to a saturated, non-aromatic, cyclic monovalent hydrocarbon radical in which one or more carbon atom(s) is/are independently replaced by the same or another heteroatom or by the same or another heteroatomic group(s). Typical heteroatoms or heteroatomic groups that may replace the carbon atoms include, but are not limited to, —O—, —S—, —N—, —Si—, —NH—, —S(O)—, —S(O)₂—, —S(O)NH—, —S(O)₂NH— and the like, and combinations thereof. Typical heteroatomic groups that may be included in these groups include, but are not limited to, —O—, —S—, —O—O—, —SS—, —O—S—, —NRR—, =NN═, —N═N—, —N═N—NRR, —PR—, —P(O)₂—, —POR—, —O—P(O)₂—, —SO—, —SO₂—, —SnR₂OR— and the like, wherein R is independently hydrogen, alkyl, substituted alkyl, aryl, substituted aryl, substituted aryl, substituted aryl and the like, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl, as defined above.

Typical heterocyclyl groups include, but are not limited to, groups derived from epoxides, azirines, thiiranes, imidazolidine, morpholine, piperazine, piperidine, pyrazolidine, pyrrolidone, quinuclidine and the like.

The heterocyclyl group can occur as a monocyclic compound, which has only a single ring, or as a polycyclic compound, which has two or more rings.

Preferably, the term “heterocyclyl” comprises a four- to seven-membered saturated heterocyclyl group comprising one, two, three or more heteroatoms selected from the group consisting of O, N and S. The heteroatom or heteroatoms may occupy any position in the heterocyclyl ring. The heteroatom or heteroatoms may occupy any position in the heterocyclyl ring.

In a preferred variant, the term “heterocyclyl” comprises a three- to seven-membered monocyclic heterocyclyl group or a nine- to twelve-membered polycyclic heterocyclyl group. In other even more preferred variants, the heterocyclyl group comprises a five-, six- or seven-membered monocyclic heterocyclyl group or a nine- to twelve-membered bicyclic heterocyclyl group. According to the invention, a monocyclic heterocyclyl group with 5 to 7 ring atoms is most preferred.

Preferred heterocyclyl groups include, but are not limited to: A five- or six-membered saturated heterocyclyl containing one or two nitrogen atoms and/or one oxygen or sulfur atom or one or two oxygen and/or sulfur atoms as ring members comprising 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-Isoxazolidinyl, 4-Isoxazolidinyl, 5-Isoxazolidinyl, 3-Isothiazolidinyl, 4-Isothiazolidinyl, 5-Isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-Imidazolidinyl, 4-Imidazolidinyl, 2-pyrrolin-2-yl, 2-pyrrolin-3-yl, 3-pyrrolin-2-yl, 3-pyrrolin-3-yl, 1-piperidinyl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl and the like.

Of the monocyclic heterocyclyl groups mentioned above, those heterocyclyl groups which are derived from the five- or six-membered saturated compounds comprising pyrrolidone, tetrahydrofuran, tetrahydrothiophene, piperidine, tetrahydropyran, tetrahydrothipyran are particularly preferred in the context of the present invention.

Of the above-mentioned polycyclic heterocyclyl ring systems, benzofuran, benzothiophene, indole, benzopyrrole, benzimidazole, benzoxazole, quinoline, quinazoline, quinoxaline or benzoxazine, 1,3-benzodioxole and benzodioxane are particularly preferred in the context of the present invention.

The heterocyclyl group or the heterocyclyl residue can be bonded to an atom of the molecule of formula (I), formula (II) or formula (III) via any ring carbon atom or ring heteroatom of the heterocyclyl group.

The heterocyclyl group, as defined above, is optionally substituted. The terms “heteroaryl” or “heteroaryl group” or “heteroaryl radical”, used equivalently side by side, by themselves or as part of another substituent according to the present invention, refer to a monovalent heteroaromatic radical obtained by removing a hydrogen atom from a single atom of a heteroaromatic ring system. Typical heteroaryl radicals or Heteroaryl groups include, but are not limited to, those groups derived from acridine, β-carboline, chroman, chromium, cinnoline, furan, imidazole, indazole, indole, indoline, indolizine, isobenzofuran, isochrome, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, thiazole, thiophene, triazole, xanthene and the like.

The heteroaryl group can occur as a monocyclic compound, which has only a single ring, or as a polycyclic compound, which has two or more rings.

Preferably, the term “heteroaryl” comprises three- to seven-membered monocyclic heteroaryl radicals comprising one, two, three or four heteroatoms selected from the group consisting of O, N and S. The heteroatom or heteroatoms may occupy any position in the heteroaryl ring. The heteroatom or heteroatoms may occupy any position in the heteroaryl ring.

In a preferred variant, the term “heteroaryl group” comprises a four- to seven-membered monocyclic heteroaryl group or a nine- to twelve-membered polycyclic heteroaryl group. In other still more preferred variants, the heteroaryl moiety comprises a five-, six- or seven-membered monocyclic heteroaryl moiety or a nine- to twelve-membered bicyclic heteroaryl moiety. According to the invention, a monocyclic C₃- to C₇-heteroaryl group is most preferred.

Particularly preferred heteroaryl radicals or heteroaryl groups include, but are not limited to, those derived from furan, thiophene, pyrrole, benzothiophene, benzofuran, benzimidazole, indole, pyridine, pyrazole, quinoline, imidazole, oxazole, isoxazole and pyrazine.

Five-membered aromatic heteroaryl radicals containing, in addition to carbon atoms, one, two or three nitrogen atoms or one or two nitrogen atoms and one sulfur or oxygen atom as ring atoms include 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-Imidazolyl, 4-Imidazolyl, and 1,3,4-triazol-2-yl.

Five-membered aromatic heteroaryl radicals containing one, two, three or four nitrogen atoms as ring atoms include 1-, 2- or 3-pyrrolyl, 1-, 3- or 4-pyrazolyl, 1-, 2- or 4-Imidazolyl, 1,2,3-[1H]-triazol-1-yl, 1,2,3-[2H]-triazol-2-yl, 1,2,3-[1H]-triazol-4-yl, 1,2,3-[1H]-triazol-5-yl, 1,2,3-[2H]-triazol-4-yl, 1,2,4-[1H]-triazol-1-yl, 1,2,4-[1H]-triazol-3-yl, 1,2,4-[1H]-triazol-5-yl, 1,2,4-[4H]-triazol-4-yl, 1,2,4-[4H]-triazol-3-yl, [1H]-tetrazol-1-yl, [1H]-tetrazol-5-yl, [2H]-tetrazol-2-yl, [2H]-tetrazol-5-yl and the like.

Five-membered aromatic heteroaryl radicals containing a heteroatom selected from oxygen or sulfur and optionally one, two or three nitrogen atoms as ring atoms include 2-furyl, 3-furyl, 2-thienyl, 3-thienyl, 3- or 4-isoxazolyl, 3- or 4-isothiazolyl, 2-, 4or 5-oxazolyl, 2-, 4 or 5-thiazolyl, 1,2,4-thiadiazol-3-yl, 1,2,4-thiadiazol-5-yl, 1,3,4-thiadiazol-2-yl, 1,2,4-oxadiazol-3-yl, 1,2,4-oxadiazol-5-yl and 1,3,4-oxadiazol-2-yl.

Six-membered heteroaryl radicals containing, in addition to carbon atoms, one or two or one, two or three nitrogen atoms as ring atoms include, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,2,4-triazin-3-yl; 1,2,4-triazin-5-yl, 1,2,4-triazin-6-yl and 1,3,5-triazin-2-yl.

Of the monocyclic heteroaryl radicals mentioned above, those heteroaryl radicals which are derived from the five- or six-membered aromatic compounds comprising pyrrole, furan, thiophene, pyridine, pyrylium ion and thiopyrylium ion, pyrazole, imidazole, imidazoline, pyrimidine, oxazole, thiazole and 1,4-thiazine are particularly preferred in the context of the present invention.

The heteroaryl group or the heteroaryl residue can be bonded to an atom of the molecule of formula (I), formula (II) or formula (III) via any ring carbon atom or ring heteroatom of the heteroaryl group.

The heteroaryl group, as defined above, is optionally substituted.

The term “salt” refers to a salt of a compound or active ingredient according to the present invention. Such salts include: (1) acid addition salts formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid 2-naphthalenesulfonic acid, 4-toluenesulfonic acid, camphorsulfonic acid, 4-methylbicyclo [2.2.2]-oct-2-ene-1-carboxylic acid, glucoheptonic acid, phenylpropionic acid, trimethylacetic acid, tertiary butylacetic acid, laurylsulfuric acid, gluconic acid, glutamic acid, hydroxynaphthoic acid, salicylic acid, stearic acid, muconic acid and the like; or (2) salts formed when an acidic proton present in the starting compound is replaced by a metal ion, e.g. an alkali metal ion, e.g. an alkali metal ion, an alkaline earth metal ion or an aluminum ion; or coordinated with an organic base such as ethanolamine, diethanolamine, triethanolamine, N-methylglucamine and the like.

The term “substituted” in the context of the present invention means that one or more hydrogen atoms of the specified group or of the specified radical or of the specified radical are independently replaced by the same or a different substituent.

The term “optionally substituted” in the context of the present invention means that the substitution is optional and not mandatory and denotes the absence or presence of a substituent or a substituent group. Thus, the term “optionally substituted XXX group” means that the XXX group may be unsubstituted but may also be substituted. For example, the term “optionally substituted alkyl” includes both substituted alkyl and unsubstituted alkyl.

The term “substituted” specifically contemplates one or more, i.e., two, three, four, five, six or more, substitutions that are common in the art. However, it is generally recognized by those skilled in the art that the substituents should be selected so as not to adversely affect the useful properties of the compound or active ingredient or its function.

In a preferred embodiment according to the present invention, the substituent or substituent group useful for substituting saturated carbon atoms in the specified group or moiety is selected from the group consisting of, but not limited to: -A, Halo, ═O, —OY, —SY, ═S, —NZZ, ═NY, ═N—OY, trihalomethyl, —CF₃, —CN, —OCN, —SCN, —NO, —N₀₂, ═N₂, —N₃, —S(O)₂Y, —S(O)₂OY, —OS(O)₂Y, —OS(O)₂OY, —P(O)(OY)₂, —P(O)(OY)(OY), —C(O)Y, —C(S)Y, —C(NY)Y, —C(O)OY, —C(S)OY, —C(O)NZZ, —C(NY)NZZ, —OC(O)Y, —OC(S)Y, —OC(O)OY, —OC(S)OY, —NYC(O)Y, —NYC(S)Y, —NYC(O)OY, —NYC(S)OY, —NYC(O)NZZ, —NYC(NY)Y or —NYC(NY)NZZ; wherein A is selected from the group consisting of optionally substituted alkyl radical, in particular C₁-C₆-alkyl radical, optionally substituted alkoxy radical, in particular C₁-C₆-alkoxy radical, optionally substituted alkylthio radical, in particular C₁-C₆-alkylthio radical, optionally substituted carbocyclyl radical, optionally substituted carboaryl radical, optionally substituted carboarylalkyl radical, optionally substituted heteroalkyl radical, optionally substituted heterocyclyl radical, optionally substituted heteroaryl radical and optionally substituted heteroarylalkyl radical, and as defined above; and/or Y means hydrogen or A; and/or Z is Y or alternatively two Z together with the nitrogen atom to which they are attached form a four, five, six or seven membered heterocyclyl or heteroaryl ring, wherein the heterocyclyl or heteroaryl ring may comprise one, two, three or four identical or different heteroatoms selected from the group consisting of nitrogen, oxygen and sulfur.

Specific examples of —NZZ should include —NH₂, —NH-alkyl, N-pyrrolidinyl and N-morpholinyl. Further specific examples include: -alkylene-O-alkyl, -alkyleneheteroaryl, -alkylene-cycloheteroalkyl, -alkylene-C(O)OY, -alkylene-C(O)NYY and —CH₂—CH₂—C(O)—CH₃, wherein Y has the meaning given above.

In a further variant, the one or more substituent group(s) together with the atoms to which they are attached may form a cyclic ring, including cycloalkyl or heterocyclyl.

Similarly, substituents or substituent groups useful for substituting unsaturated carbon atoms in the previously disclosed groups or the disclosed radical include, but are not limited to, -A, Halo, ═O, —OY, —SY, ═S, —NZZ, ═NY, ═N—OY, trihalomethyl, —CF₃, —CN, —OCN, —SCN, —NO, —NO₂, ═N₂, —N₃, —S(O)₂Y, —S(O)₂OY, —OS(O)₂Y, —OS(O)₂OY, —P(O)(OY)₂, —P(O)(OY)(OY), —C(O)Y, —C(S)Y, —C(NY)Y, —C(O)OY, —C(S)OY, —C(O)NZZ, —C(NY)NZZ, —OC(O)Y, —OC(S)Y, —OC(O)OY, —OC(S)OY, —NYC(O)Y, —NYC(S)Y, —NYC(O)OY, —NYC(S)OY, —NYC(O)NZZ, —NYC(NY)Y and —NYC(NY)NZZ, wherein A, Y and Z have the same meaning as defined above.

Substituents or substituent groups for the substitution of nitrogen atoms heterocyclyl groups, without being limited thereto, -A, —OY, —SY, —NZZ, trihalomethyl, —CF₃, —CN, —OCN, —SCN, —NO, —NO₂, —S(O)₂Y, —S(O)₂OY, —OS(O)₂Y, —OS(O)₂OY, —P(O)(OY)₂, —P(O)(OY)(OY), —C(O)Y, —C(S)Y, —C(NY)Y, —C(O)OY, —C(S)OY, —C(O)NZZ, —C(NY)NZZ, —OC(O)Y, —OC(S)Y, —OC(O)OY, —OC(S)OY, —NYC(O)Y, —NYC(S)Y, —NYC(O)OY, —NYC(S)OY, —NYC(O)NZZ, —NYC(NY)Y and —NYC(NY)NZZ, wherein A, Y and Z have the same meaning as defined above.

Suitable substituents within the scope of the present invention preferably include halogen groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy groups or heteroaryloxy groups, arylalkyl or heteroarylalkyl groups, arylalkoxy or heteroarylalkoxy groups, amino groups, alkyl and dialkylamino groups, carbamoyl groups, alkylcarbonyl groups, carboxyl groups, alkoxycarbonyl groups, alkylaminocarbonyl groups, dialkylaminocarbonyl groups, arylcarbonyl groups, aryloxycarbonyl groups, alkylsulphonyl groups, arylsulphonyl groups, cycloalkyl groups, cyano groups, C₁- to C₆-alkylthio groups, arylthio groups, nitro groups, keto groups, acyl groups, boronate or boronyl groups, phosphate or phosphonyl groups, sulphamyl groups, sulphonyl groups, sulphinyl groups and combinations thereof. In the case of substituted combinations such as substituted arylalkyl, either the aryl or the alkyl group may be substituted, or both the aryl and the alkyl group may be substituted with one or more substituents.

Preferred substituents for the above-mentioned groups or radicals are in particular selected from COOH, COO-alkyl, NH₂, NO₂, OH, SH, CN, Si, halogens, linear or branched C₁- to C₆-alkyl groups, linear or branched C₁- to C₆-alkyloxy groups or linear or branched C₁- to C₆-alkylthio groups, where one or more H atoms in the alkyl groups may be replaced by halogen.

In addition, in some cases suitable substituents may be combined to form one or more rings as known to those skilled in the art.

According to the invention, the substituents or substituent groups of the groups or radicals defined above are each selected independently of one another.

In accordance with the invention, the substituents used to substitute a particular group or residue or radical may in turn be further substituted, typically with one or more of the same or different groups or radicals selected from the various groups or radicals disclosed above and as defined in detail above.

Surprisingly, it was found that the compounds or active ingredients according to the invention have the common property of stimulating the trigeminalnerve, even at low doses, and that the active ingredients produce a salivation-promoting and/or tingling/fizzing effect when consumed or applied. The salivation-promoting effect is particularly pronounced with the active ingredients according to the invention. This means that in the final preparation, even a lower dosage of the active ingredient according to the invention or a mixture of active ingredients according to the invention is sufficient to cause an intense mouth-watering and/or tingling effect when used or consumed. Thus, the compounds described herein are particularly efficient active ingredients.

The compounds or active ingredients according to the invention are further characterized by the fact that they are largely tasteless and odorless, so that they are also excellently suited for incorporation into neutral and/or flavored preparations without creating a taste impression that is perceived as negative, for example as bitter or metallic, or adversely affecting the intended taste impression.

In addition, the active ingredients according to the invention are characterized by the fact that when they are used or consumed, the trigeminal effect, in particular the mouth-watering effect, sets in quickly and immediately and lasts for a long time.

The active ingredients according to the invention are also colorless and non-discoloring, which is particularly advantageous for their storage and/or use in the end product.

Equally surprising was the fact that the active ingredients according to the invention are able to reduce or mask the known taste disadvantages of flavors, especially of sweeteners such as steviosides, such as off-notes and/or off-flavors. In particular, bitter, burning, astringent, dry, dusty, floury, chalky or metallic flavors are reduced or neutralized even when small amounts of an active ingredient according to the invention are added.

In an even more preferred variant according to the first aspect of the present invention, it is an active ingredient wherein in the general formula (I)
R1, R4 and R5 each represent H independently of each other;
R2 and/or R3 are identical or different and each independently represents H, OH or an optionally substituted C₁-, C₂-, C₃- or C₄-alkoxy group;
X stands for an optionally substituted C₁-, C₂-, C₃- or C₄-alkyl group or an optionally substituted C₁-, C₂-, C₃- or C₄-alkenyl group or an optionally substituted C₁—, C₂—, C₃- or C₄-alkoxy group;
R7 represents an optionally substituted five or six membered cycloalkyl group or an optionally substituted five or six membered aryl group or an optionally substituted five or six membered heterocyclyl group having one, two or more identical or different heteroatom(s) or an optionally substituted five or six membered heteroaryl group having one, two or more identical or different heteroatom(s), wherein the heteroatoms are selected from the group consisting of nitrogen, oxygen and sulfur;
optionally one or both nitrogen atom(s) of the saturated six-membered ring in the general formula (I) is/are replaced by a C atom;
optionally the saturated six-membered ring in the general formula (I) is mono- or polysubstituted; or
   wherein in the general formula (II)
R1, R4 and R5 each represent H independently of each other;
R2 and/or R3 are identical or different and each independently represents H, OH or an optionally substituted C₁-, C₂-, C₃- or C₄-alkoxy group;
X stands for an optionally substituted C₁-, C₂-, C₃- or C₄-alkyl group or an optionally substituted C₁-, C₂-, C₃- or C₄-alkenyl group or an optionally substituted C₁—, C₂—, C₃- or C₄-alkoxy group;
R7 represents an optionally substituted five or six membered cycloalkyl group or an optionally substituted five or six membered aryl group or an optionally substituted five or six membered heterocyclyl group having one, two or more identical or different heteroatom(s) or an optionally substituted five or six membered heteroaryl group having one, two or more identical or different heteroatom(s), wherein the heteroatoms are selected from the group consisting of nitrogen, oxygen and sulfur;
optionally the nitrogen atom of the saturated six-membered ring in the general formula (II) is replaced by a C atom;
optionally the saturated six-membered ring in the general formula (II) is mono- or polysubstituted; or
   wherein in the general formula (III)
R1, R4 and R5 each represent H independently of each other;
R2 and/or R3 are identical or different and each independently represents H, OH or an optionally substituted C₁-, C₂-, C₃- or C₄-alkoxy group;
X stands for an optionally substituted C₁-, C₂-, C₃- or C₄-alkyl group or an optionally substituted C₁-, C₂-, C₃- or C₄-alkenyl group or an optionally substituted C₁—, C₂—, C₃- or C₄-alkoxy group;
R7 represents an optionally substituted five or six membered cycloalkyl group or an optionally substituted five or six membered aryl group or an optionally substituted five or six membered heterocyclyl group having one, two or more identical or different heteroatom(s) or an optionally substituted five or six membered heteroaryl group having one, two or more identical or different heteroatom(s), wherein the heteroatoms are selected from the group consisting of nitrogen, oxygen and sulfur;
optionally one or both nitrogen atom(s) of the saturated seven-membered ring in the general formula (III) is/are replaced by a C atom;
optionally the saturated seven-membered ring in the general formula (III) is mono- or polysubstituted.

Even more preferred according to the invention are active compounds wherein in the general formula (I)
R1, R4 and R5 each represent H independently of each other;
R2 and/or R3 are identical or different and each independently represents H, OH or an optionally substituted C₁—, C₂- or C₃-alkoxy group;
X stands for an optionally substituted C₁-, C₂-, C₃- or C₄-alkyl group or an optionally substituted C₁-, C₂-, C₃- or C₄-alkenyl group or an optionally substituted C₁—, C₂—, C₃- or C₄-alkoxy group;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally one or both nitrogen atom(s) of the saturated six-membered ring in the general formula (I) is/are replaced by a C atom;
optionally the saturated six-membered ring in the general formula (I) is mono- or polysubstituted; or
   wherein in the general formula (II)
R1, R4 and R5 each represent H independently of each other;
R2 and/or R3 are identical or different and each independently represents H, OH or an optionally substituted C₁—, C₂- or C₃-alkoxy group;
X stands for an optionally substituted C₁-, C₂-, C₃- or C₄-alkyl group or an optionally substituted C₁-, C₂-, C₃- or C₄-alkenyl group or an optionally substituted C₁—, C₂—, C₃- or C₄-alkoxy group;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally the nitrogen atom of the saturated six-membered ring in the general formula (II) is replaced by a C atom;
optionally the saturated six-membered ring in the general formula (II) is mono- or polysubstituted; or
   wherein in the general formula (III)
R1, R4 and R5 each represent H independently of each other;
R2 and/or R3 are identical or different and each independently represents OH or an optionally substituted C₁—, C₂- or C₃-alkoxy group;
X stands for an optionally substituted C₁-, C₂-, C₃- or C₄-alkyl group or an optionally substituted C₁-, C₂-, C₃- or C₄-alkenyl group or an optionally substituted C₁—, C₂—, C₃- or C₄-alkoxy group;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally one or both nitrogen atom(s) of the saturated seven-membered ring in the general formula (III) is/are replaced by a C atom;
optionally the saturated seven-membered ring in the general formula (III) is mono- or polysubstituted.

Active ingredients with particularly advantageous properties, i.e., a particularly intensive and effective mouth-watering and/or tingling/fizzing effect, are regularly found in structures/active ingredients in which in the general formula (I) or the general formula (II) or the general formula (III)
R1, R4 and R5 each represent H independently of each other;
R2 and/or R3 are the same or different and each independently represents H or OH or C1-alkoxy or C2-alkoxy or C3-alkoxy and X represents C3-alkenyl; or
R2 and/or R3 are the same or different and each independently represents H or OH or C1-alkoxy or C2-alkoxy or C3-alkoxy and X represents C4-alkyl; or
R2 and/or R3 are the same or different and each independently represents H or OH or C1-alkoxy or C2-alkoxy or C3-alkoxy and X represents C3-alkyl; or
R2 and/or R3 are the same or different and each independently represents H or OH or C1-alkoxy or C2-alkoxy or C3-alkoxy and X represents C2-alkyl; or
R2 and/or R3 are the same or different and each independently represents H or OH or C1-alkoxy or C2-alkoxy or C3-alkoxy and X represents C1-alkyl; or
R2 and/or R3 are the same or different and each independently represents H or OH or C1-alkoxy or C2-alkoxy or C3-alkoxy and X represents C2-alkoxy;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally one or both nitrogen atom(s) of the saturated six-membered ring in the general formula (I) or the nitrogen atom of the saturated six-membered ring in the general formula (II) or one or both nitrogen atom(s) of the saturated seven-membered ring in the general formula (III) is/are replaced by a C atom;
optionally the saturated six-membered ring in the general formula (I) or in the general formula (II) or the saturated seven-membered ring in the general formula (III) is mono- or polysubstituted.

In an even more preferred variant according to the first aspect of the present invention, it is an active compound, wherein in the general formula (I)
R1, R4 and R5 each independently represent H; and
R2 and/or R3 are the same or different and each independently represents H, OH, C1-alkoxy, C2-alkoxy or C3-alkoxy and X represents C2-alkyl, C3-alkyl, C4-alkyl, C3-alkenyl or C2-alkoxy;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally one or both nitrogen atom(s) of the saturated six-membered ring in the general formula (I) is/are replaced by a C atom;
optionally the saturated six-membered ring in the general formula (I) is mono- or polysubstituted; or
   wherein in the general formula (II)
R1, R4 and R5 each represent H independently of each other;
R2 and/or R3 are the same or different and each independently represents H, C1-alkoxy or C2-alkoxy and X represents C3-alkyl;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally the nitrogen atom of the saturated six-membered ring in the general formula (II) is replaced by a C atom;
optionally the saturated six-membered ring in the general formula (II) is mono- or polysubstituted; or
   wherein in the general formula (III)
R1, R4 and R5 each represent H independently of each other;
R2 and/or R3 are the same or different and each independently represents H, C1-alkoxy or C2-alkoxy and X represents C2-alkyl;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally one or both nitrogen atom(s) of the saturated seven-membered ring in the general formula (III) is/are replaced by a C atom;
optionally the saturated six-membered ring in the general formula (III) is mono- or polysubstituted.

In an even more preferred variant according to the first aspect of the present invention, it is an active compound, wherein in the general formula (I)
R1, R4 and R5 each represent H independently of each other;
R2, R3 and X have the following options:

| Option | R2 | R3 | X |
|---|---|---|---|
| No. 1 | Propoxy | Propoxy | Propenyl |
| No. 2 | Methoxy | OH | Propenyl |
| No. 3 | Propoxy | Propoxy | Propenyl |
| No. 4 | OH | Methoxy | Propenyl |
| No. 5 | Propoxy | Propoxy | Propenyl |
| No. 6 | Ethoxy | Ethoxy | Propyl |
| No. 7 | Ethoxy | Ethoxy | Ethyl |
| No. 8 | Ethoxy | Ethoxy | Ethoxy |
| No. 9 | H | H | Ethyl |
| No. 10 | Ethoxy | Ethoxy | Ethyl |
| No. 11 | Methoxy | Methoxy | Butyl |
| No. 12 | Methoxy | Methoxy | Ethyl |
| No. 13 | H | Methoxy | Butyl |
| No. 14 | H | Methoxy | Ethyl |
| No. 15 | H | Ethoxy | Butyl |
| No. 16 | H | Ethoxy | Ethyl |

R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally one or both nitrogen atom(s) of the saturated six-membered ring in the general formula (I) is/are replaced by a C atom;
optionally the saturated six-membered ring in the general formula (I) is mono- or polysubstituted; or
   wherein in the general formula (II)
R1, R4 and R5 each represent H independently of each other;
R2, R3 and X have the following options:

| Option | R2 | R3 | X |
|---|---|---|---|
| No. 40 | Ethoxy | Ethoxy | Propyl |
| No. 41 | Methoxy | Methoxy | Propyl |
| No. 42 | H | Methoxy | Propyl |
| No. 43 | H | Ethoxy | Propyl |

R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally the nitrogen atom of the saturated six-membered ring in the general formula (II) is replaced by a C atom;
optionally the saturated six-membered ring in the general formula (II) is mono- or polysubstituted; or
   wherein in the general formula (III)
R1, R4 and R5 each represent H independently of each other;
R2, R3 and X have the following options:

| Option | R2 | R3 | X |
|---|---|---|---|
| No. 45 | Ethoxy | Ethoxy | Ethyl |
| No. 46 | Methoxy | Methoxy | Ethyl |
| No. 47 | H | Methoxy | Ethyl |
| No. 48 | H | Ethoxy | Ethyl |

R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally one or both nitrogen atom(s) of the saturated seven-membered ring in the general formula (III) is/are replaced by a C atom;
optionally the saturated seven-membered ring in the general formula (III) is mono- or polysubstituted.

In an even more preferred variant according to the first aspect of the present invention, it is an active ingredient, wherein the active ingredient of the general formula (I) is selected from the group consisting of:

TABLE 1

| Comp. | | | |
|---|---|---|---|
| No. | | Compound | Chemical name |
| 1 | 1 | 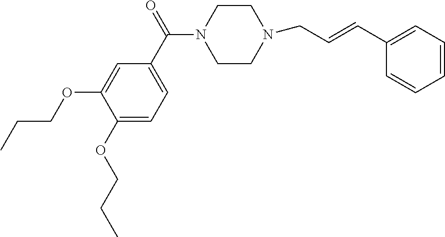 [SMILES: CCCOc1ccc(C(=O)N2CCN(C/C=C/c3ccccc3)CC2)cc1OCCC] | [4-[(E)-cinnamyl]piperazin-1- yl]-(3,4-dipropoxy- phenyl)methanone |
| 2 | 4 | 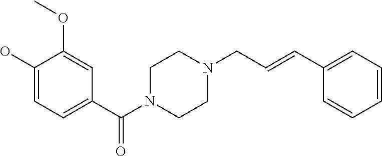 [SMILES: COc1cc(C(=O)N2CCN(C/C=C/c3ccccc3)CC2)ccc1O] | [4-[(E)-cinnamyl]piperazin-1- yl]-(4-hydroxy-3-methoxy- phenyl)methanone |
| 3 | 6 | 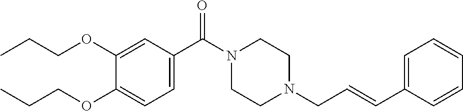 [SMILES: CCCOc1ccc(C(=O)N2CCN(C/C=C/c3ccccc3)CC2)cc1OCCC] | [4-[(E)-cinnamyl]piperazin-1- yl]-(3,4-dipropoxy- phenyl)methanone |
| 4 | 9 | 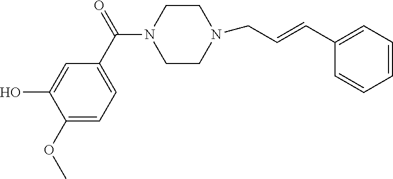 [SMILES: COc1ccc(C(=O)N2CCN(C/C=C/c3ccccc3)CC2)cc1O] | [4-[(E)-cinnamyl]piperazin-1- yl]-(3-hydroxy-4-methoxy- phenyl)methanone |
| 5 | 10 | 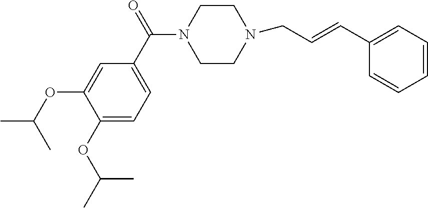 [SMILES: CC(C)Oc1ccc(C(=O)N2CCN(C/C=C/c3ccccc3)CC2)cc1OC(C)C] | [4-[(E)-cinnamyl]piperazin-1- yl]-(3,4-diisopropoxy- phenyl)methanone |
| 6 | 11 | 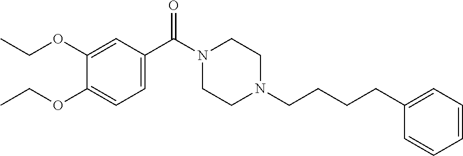 [SMILES: CCOc1ccc(C(=O)N2CCN(CCCCc3ccccc3)CC2)cc1OCC] | (3,4-Diethoxyphenyl)-[4-(4- phenylbutyl)piperazin-1- yl]methanone |
| 7 | 12 | 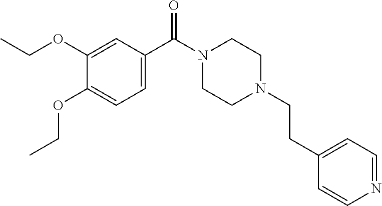 [SMILES: CCOc1ccc(C(=O)N2CCN(CCc3ccncc3)CC2)cc1OCC] | (3,4-Diethoxyphenyl)-[4-[2-(4- pyridyl)ethyl]piperazin-1- yl]methanone |
| 8 | 13 | 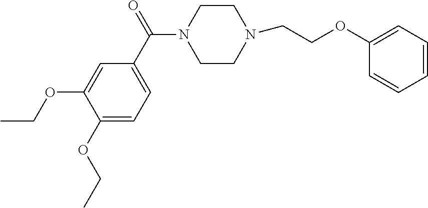 [SMILES: CCOc1ccc(C(=O)N2CCN(CCOc3ccccc3)CC2)cc1OCC] | (3,4-Diethoxyphenyl)-[4-(2- phenoxyethyl)piperazin-1- yl]methanone |
| 9 | 19 | 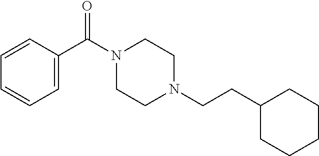 [SMILES: O=C(c1ccccc1)N1CCN(CCC2CCCCC2)CC1] | [4-(2-cyclohexylethyl) piperazin-1-yl]-phenyl-methanone |
| 10 | 24 | 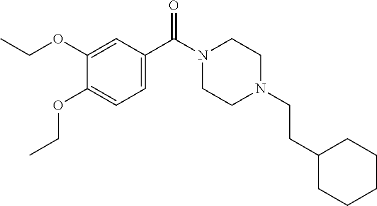 [SMILES: CCOc1ccc(C(=O)N2CCN(CCC3CCCCC3)CC2)cc1OCC] | [4-(2-cyclohexylethyl) piperazin-1-yl]-(3,4-diethoxy- phenyl)methanone |
| 11 | 27 | 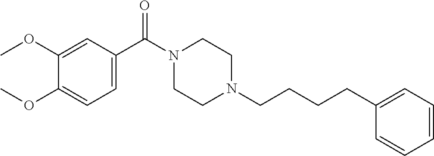 [SMILES: COc1ccc(C(=O)N2CCN(CCCCc3ccccc3)CC2)cc1OC] | (3,4-Dimethoxyphenyl)-[4-(4- phenylbutyl)piperazin-1- yl]methanone |
| 12 | 29 | 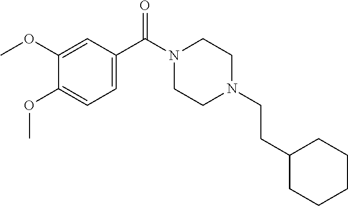 [SMILES: COc1ccc(C(=O)N2CCN(CCC3CCCCC3)CC2)cc1OC] | [4-(2-cyclohexylethyl) piperazin-1-yl]-(3,4-dimethoxy- phenyl)methanone |
| 13 | 34 | 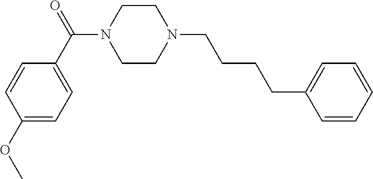 [SMILES: COc1ccc(C(=O)N2CCN(CCCCc3ccccc3)CC2)cc1] | (4-Methoxyphenyl)-[4-(4- phenylbutyl)piperazin-1-yl] methanone |
| 14 | 36 | 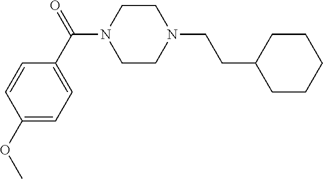 [SMILES: COc1ccc(C(=O)N2CCN(CCC3CCCCC3)CC2)cc1] | [4-(2-cyclohexylethyl) piperazin-1-yl]-(4-methoxy- phenyl)methanone |
| 15 | 41 | 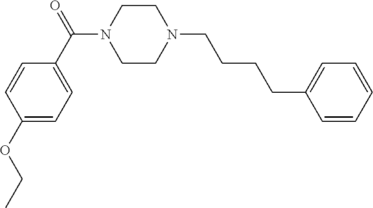 [SMILES: CCOc1ccc(C(=O)N2CCN(CCCCc3ccccc3)CC2)cc1] | (4-ethoxyphenyl)-[4-(4- phenylbutyl)piperazin-1-yl] methanone |
| 16 | 44 | 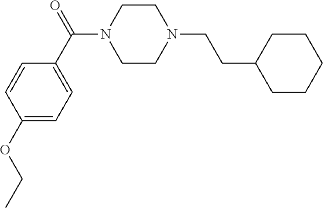 [SMILES: CCOc1ccc(C(=O)N2CCN(CCC3CCCCC3)CC2)cc1] | [4-(2-cyclohexylethyl) piperazin-1-yl]-(4-ethoxy- phenyl)methanone |
| 17 | 2 | 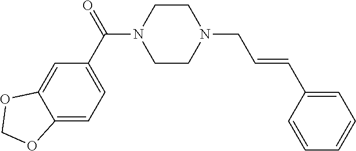 [SMILES: O=C(c1ccc2c(c1)OCO2)N1CCN(C/C=C/c2ccccc2)CC1] | 1,3-benzodioxol-5-yl-[4-[(E)- cinnamyl]piperazin-1-yl] methanone |
| 18 | 3 | 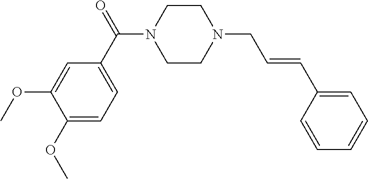 [SMILES: COc1ccc(C(=O)N2CCN(C/C=C/c3ccccc3)CC2)cc1OC] | [4-[(E)-cinnamyl]piperazin-1- yl]-(3,4-dimethoxy- phenyl)methanone |
| 19 | 5 | 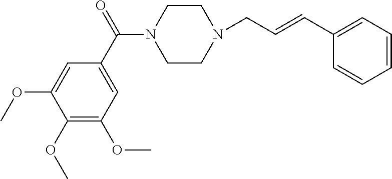 [SMILES: COc1cc(C(=O)N2CCN(C/C=C/c3ccccc3)CC2)cc(OC)c1OC] | [4-[(E)-cinnamyl]piperazin-1- yl]-(3,4,5-trimethoxy- phenyl)methanone |
| 20 | 7 | 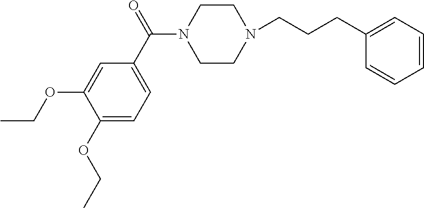 [SMILES: CCOc1ccc(C(=O)N2CCN(CCCc3ccccc3)CC2)cc1OCC] | (3,4-Diethoxyphenyl)-[4-(3- phenylpropyl)piperazin-1- yl]methanone |
| 21 | 8 | 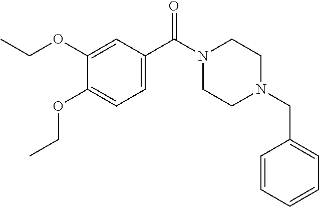 [SMILES: CCOc1ccc(C(=O)N2CCN(Cc3ccccc3)CC2)cc1OCC] | (4-benzylpiperazin-1-yl)-(3,4- diethoxyphenyl)methanone |
| 22 | 14 | 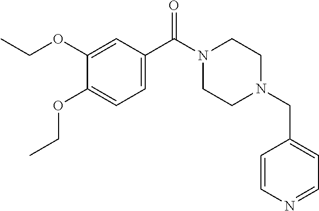 [SMILES: CCOc1ccc(C(=O)N2CCN(Cc3ccncc3)CC2)cc1OCC] | (3,4-Diethoxyphenyl)-[4-(4- pyridylmethyl)piperazin-1- yl]methanone |
| 23 | 15 | 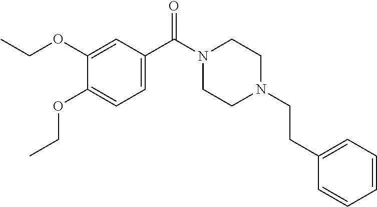 [SMILES: CCOc1ccc(C(=O)N2CCN(CCc3ccccc3)CC2)cc1OCC] | (3,4-Diethoxyphenyl)-[4-(2- phenylethyl)piperazin-1- yl]methanone |
| 24 | 16 | 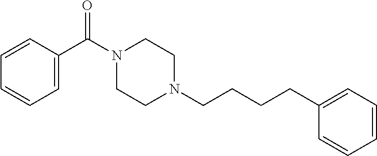 [SMILES: O=C(c1ccccc1)N1CCN(CCCCc2ccccc2)CC1] | Phenyl-[4-(4-phenylbutyl) piperazin-1-yl]methanone |
| 25 | 17 | 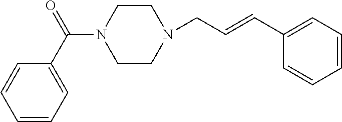 [SMILES: O=C(c1ccccc1)N1CCN(C/C=C/c2ccccc2)CC1] | [4-[(E)-cinnamyl]piperazin-1- yl]-phenyl-methanone |
| 26 | 18 | 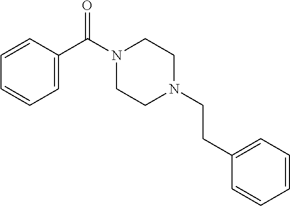 [SMILES: O=C(c1ccccc1)N1CCN(CCc2ccccc2)CC1] | Phenyl-[4-(2-phenylethyl) piperazin-1-yl]methanone |
| 27 | 21 | 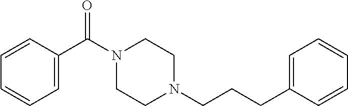 [SMILES: O=C(c1ccccc1)N1CCN(CCCc2ccccc2)CC1] | Phenyl-[4-(3-phenylpropyl) piperazin-1-yl]methanone |
| 28 | 23 | 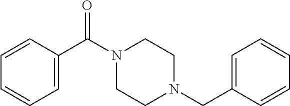 [SMILES: O=C(c1ccccc1)N1CCN(Cc2ccccc2)CC1] | (4-benzylpiperazin-1-yl)- phenyl-methanone |
| 29 | 28 | 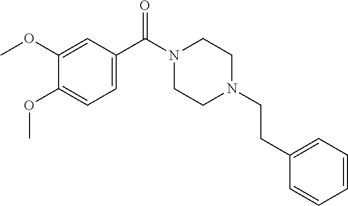 [SMILES: COc1ccc(C(=O)N2CCN(CCc3ccccc3)CC2)cc1OC] | (3,4-Dimethoxyphenyl)-[4-(2- phenylethyl)piperazin-1- yl]methanone |
| 30 | 31 | 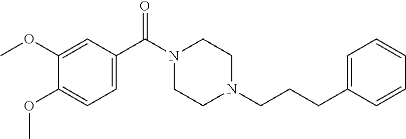 [SMILES: COc1ccc(C(=O)N2CCN(CCCc3ccccc3)CC2)cc1OC] | (3,4-Dimethoxyphenyl)-[4-(3- phenylpropyl)piperazin-1- yl]methanone |
| 31 | 33 | 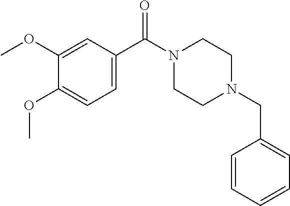 [SMILES: COc1ccc(C(=O)N2CCN(Cc3ccccc3)CC2)cc1OC] | (4-benzylpiperazin-1-yl)-(3,4- dimethoxyphenyl)methanone |
| 32 | 35 | 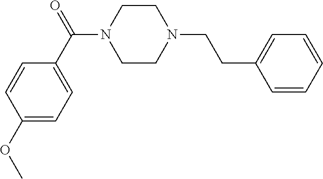 [SMILES: COc1ccc(C(=O)N2CCN(CCc3ccccc3)CC2)cc1] | (4-Methoxyphenyl)-[4-(2- phenylethyl)piperazin-1-yl] methanone |
| 33 | 38 | 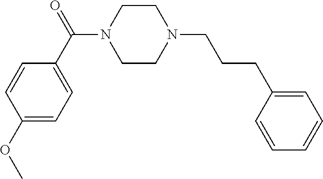 [SMILES: COc1ccc(C(=O)N2CCN(CCCc3ccccc3)CC2)cc1] | (4-Methoxyphenyl)-[4-(3- phenylpropyl)piperazin-1-yl] methanone |
| 34 | 40 | 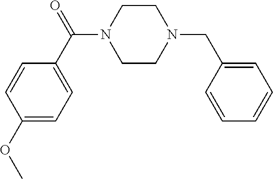 [SMILES: COc1ccc(C(=O)N2CCN(Cc3ccccc3)CC2)cc1] | (4-benzylpiperazin-1-yl)-(4- methoxyphenyl)methanone |
| 35 | 42 | 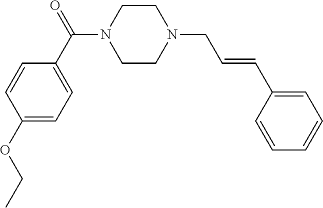 [SMILES: CCOc1ccc(C(=O)N2CCN(C/C=C/c3ccccc3)CC2)cc1] | [4-[(E)-cinnamyl]piperazin-1- yl]-(4-ethoxyphenyl)methanone |
| 36 | 43 | 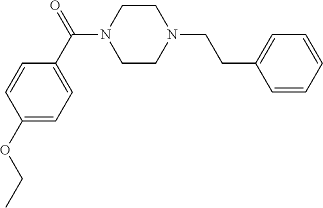 [SMILES: CCOc1ccc(C(=O)N2CCN(CCc3ccccc3)CC2)cc1] | (4-ethoxyphenyl)-[4-(2- phenylethyl)piperazin-1-yl] methanone |
| 37 | 46 | 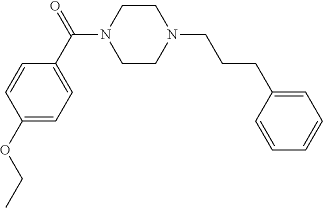 [SMILES: CCOc1ccc(C(=O)N2CCN(CCCc3ccccc3)CC2)cc1] | (4-ethoxyphenyl)-[4-(3- phenylpropyl)piperazin-1-yl] methanone |
| 38 | 48 | 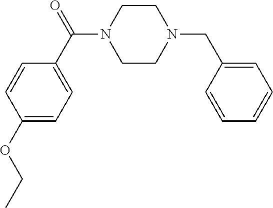 [SMILES: CCOc1ccc(C(=O)N2CCN(Cc3ccccc3)CC2)cc1] | (4-benzylpiperazin-1-yl)-(4- ethoxyphenyl)methanone |

or the active ingredient of the general formula (II) is selected from the group consisting of

TABLE 2

| Comp. | | | |
|---|---|---|---|
| No. | | Compound | Chemical name |
| 39 | 20 | 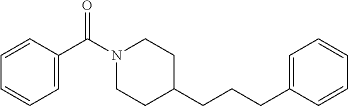 [SMILES: O=C(c1ccccc1)N1CCC(CCCc2ccccc2)CC1] | Phenyl-[4-(3-phenylpropyl)-1- piperidyl]methanone |
| 40 | 25 | 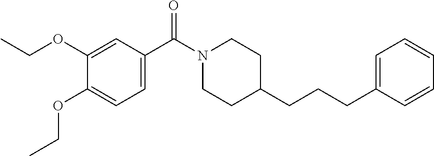 [SMILES: CCOc1ccc(C(=O)N2CCC(CCCc3ccccc3)CC2)cc1OCC] | (3,4-Diethoxyphenyl)-[4-(3- phenylpropyl)-1- piperidyl]methanone |
| 41 | 30 | 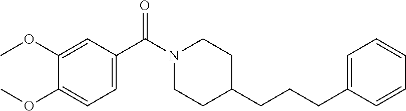 [SMILES: COc1ccc(C(=O)N2CCC(CCCc3ccccc3)CC2)cc1OC] | (3,4-Dimethoxyphenyl)-[4-(3- phenylpropyl)-1- piperidyl]methanone |
| 42 | 37 | 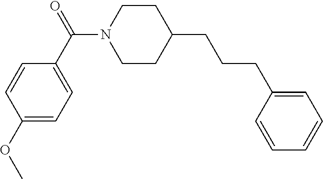 [SMILES: COc1ccc(C(=O)N2CCC(CCCc3ccccc3)CC2)cc1] | (4-Methoxyphenyl)-[4-(3- phenylpropyl)-1-piperidyl] methanone |
| 43 | 45 | 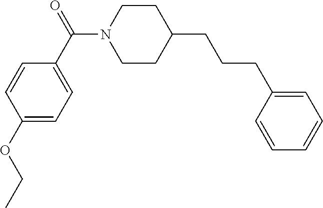 [SMILES: CCOc1ccc(C(=O)N2CCC(CCCc3ccccc3)CC2)cc1] | (4-ethoxyphenyl)-[4-(3- phenylpropyl)-1-piperidyl] methanone |

or the active ingredient of the general formula (III) is selected from the group consisting of

TABLE 3

| Comp. | | | |
|---|---|---|---|
| No. | | Compound | Chemical name |
| 44 | 22 | 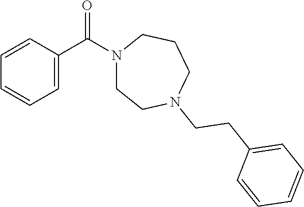 [SMILES: O=C(c1ccccc1)N1CCCN(CCc2ccccc2)CC1] | Phenyl-[4-(2-phenylethyl)-1,4- diazepan-1-yl]methanone |
| 45 | 26 | 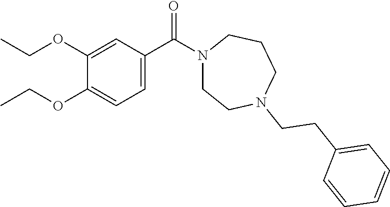 [SMILES: CCOc1ccc(C(=O)N2CCCN(CCc3ccccc3)CC2)cc1OCC] | (3,4-Diethoxyphenyl)-[4-(2- phenylethyl)-1,4-diazepan-1- yl]methanone |
| 46 | 32 | 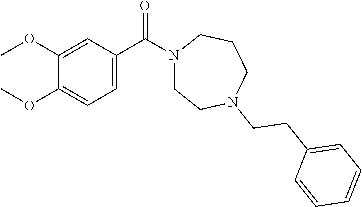 [SMILES: COc1ccc(C(=O)N2CCCN(CCc3ccccc3)CC2)cc1OC] | (3,4-Dimethoxyphenyl)-[4-(2- phenylethyl)-1,4-diazepan-1- yl]methanone |
| 47 | 39 | 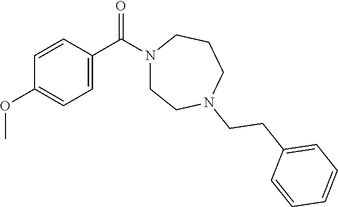 [SMILES: COc1ccc(C(=O)N2CCCN(CCc3ccccc3)CC2)cc1] | (4-Methoxyphenyl)-[4-(2- phenylethyl)-1,4-diazepan-1- yl]methanone |
| 48 | 47 | 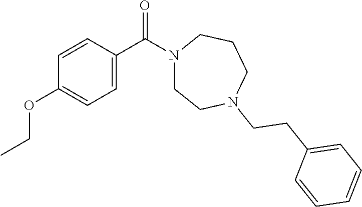 [SMILES: CCOc1ccc(C(=O)N2CCCN(CCc3ccccc3)CC2)cc1] | (4-ethoxyphenyl)-[4-(2- phenylethyl)-1,4-diazepan-1- yl]methanone |

as well as their salts, in particular acid addition salts, with inorganic or organic acids, whereby the active ingredients may be present in stereonisomericaly pure form or as mixtures of different stereoisomers.

In a still further preferred variant according to the first aspect of the present invention, it is an active ingredient, wherein the active ingredient of the general formula (I) is selected from the group consisting of:

TABLE 4

| Comp. | | | |
|---|---|---|---|
| No. | | Compound | Chemical name |
| 1 | 1 | 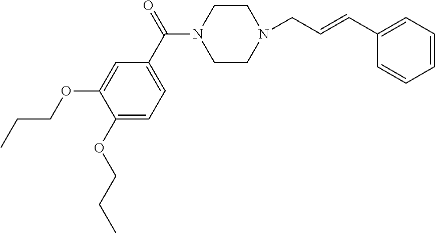 [SMILES: CCCOc1ccc(C(=O)N2CCN(C/C=C/c3ccccc3)CC2)cc1OCCC] | [4-[(E)-cinnamyl]piperazin-1- yl]-3,4-dipropoxy- phenyl)methanone |
| 6 | 11 | 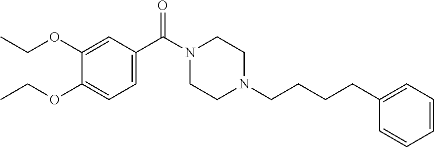 [SMILES: CCOc1ccc(C(=O)N2CCN(CCCCc3ccccc3)CC2)cc1OCC] | (3,4-Diethoxyphenyl)-[4-(4- phenylbutyl)piperazin-1- yl]methanone |
| 7 | 12 | 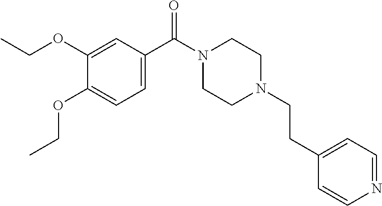 [SMILES: CCOc1ccc(C(=O)N2CCN(CCc3ccncc3)CC2)cc1OCC] | (3,4-Diethoxyphenyl)-[4-[2-(4- pyridyl)ethyl]piperazin-1- yl]methanone |
| 8 | 13 | 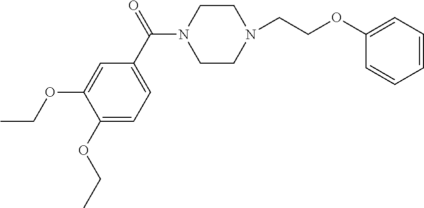 [SMILES: CCOc1ccc(C(=O)N2CCN(CCOc3ccccc3)CC2)cc1OCC] | (3,4-Diethoxyphenyl)-[4-(2- phenoxyethyl)piperazin-1- yl]methanone |
| 9 | 19 | 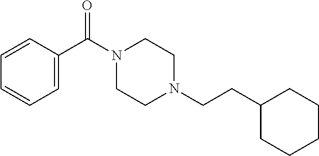 [SMILES: O=C(c1ccccc1)N1CCN(CCC2CCCCC2)CC1] | [4-(2-cyclohexylethyl)piperazin- 1-yl]-phenyl-methanone |
| 11 | 27 | 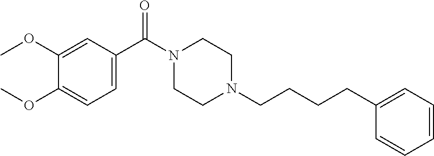 [SMILES: COc1ccc(C(=O)N2CCN(CCCCc3ccccc3)CC2)cc1OC] | (3,4-Dimethoxyphenyl)-[4-(4- phenylbutyl)piperazin-1- yl]methanone |
| 13 | 34 | 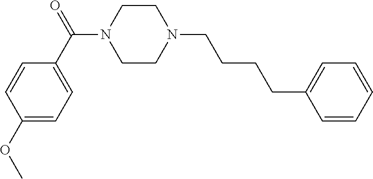 [SMILES: COc1ccc(C(=O)N2CCN(CCCCc3ccccc3)CC2)cc1] | (4-Methoxyphenyl)-[4-(4- phenylbutyl)piperazin-1-yl] methanone |
| 14 | 36 | 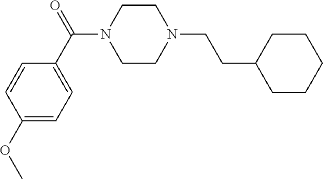 [SMILES: COc1ccc(C(=O)N2CCN(CCC3CCCCC3)CC2)cc1] | [4-(2-cyclohexylethyl) piperazin-1-yl]-(4-methoxy- phenyl)methanone |
| 15 | 41 | 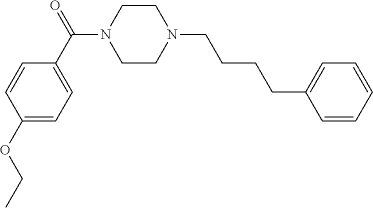 [SMILES: CCOc1ccc(C(=O)N2CCN(CCCCc3ccccc3)CC2)cc1] | (4-ethoxyphenyl)-[4-(4- phenylbutyl)piperazin-1-yl] methanone |
| 16 | 44 | 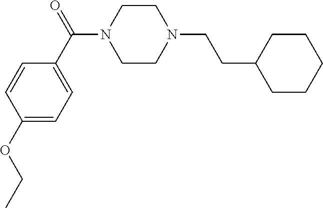 [SMILES: CCOc1ccc(C(=O)N2CCN(CCC3CCCCC3)CC2)cc1] | [4-(2-cyclohexylethyl) piperazin-1-yl]-(4-ethoxy- phenyl)methanone |
| 18 | 3 | 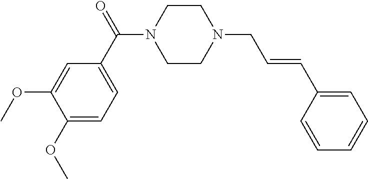 [SMILES: COc1ccc(C(=O)N2CCN(C/C=C/c3ccccc3)CC2)cc1OC] | [4-[(E)-cinnamyl]piperazin-1- yl]-(3,4-dimethoxy- phenyl)methanone |
| 20 | 7 | 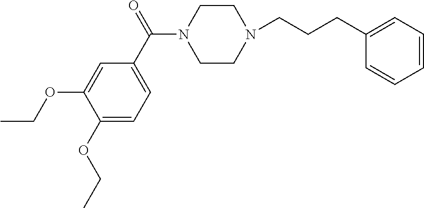 [SMILES: CCOc1ccc(C(=O)N2CCN(CCCc3ccccc3)CC2)cc1OCC] | (3,4-Diethoxyphenyl)-[4-(3- phenylpropyl)piperazin-1- yl]methanone |
| 21 | 8 | 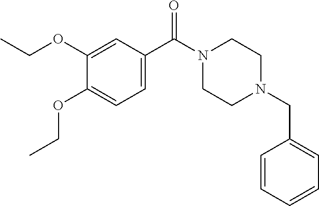 [SMILES: CCOc1ccc(C(=O)N2CCN(Cc3ccccc3)CC2)cc1OCC] | (4-benzylpiperazin-1-yl)-(3,4- diethoxyphenyl)methanone |
| 22 | 14 | 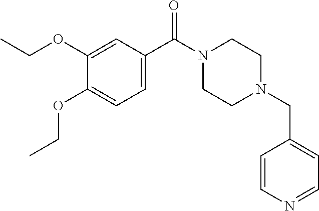 [SMILES: CCOc1ccc(C(=O)N2CCN(Cc3ccncc3)CC2)cc1OCC] | (3,4-Diethoxyphenyl)-[4-(4- pyridylmethyl)piperazin-1- yl]methanone |
| 23 | 15 | 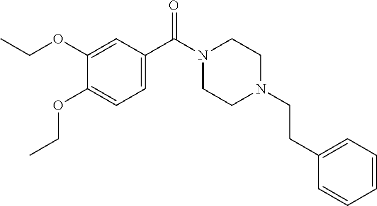 [SMILES: CCOc1ccc(C(=O)N2CCN(CCc3ccccc3)CC2)cc1OCC] | (3,4-Diethoxyphenyl)-[4-(2- phenylethyl)piperazin-1- yl]methanone |
| 25 | 17 | 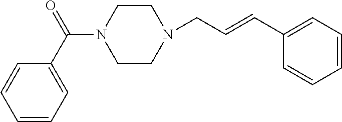 [SMILES: O=C(c1ccccc1)N1CCN(C/C=C/c2ccccc2)CC1] | [4-[(E)-cinnamyl]piperazin-1- yl]-phenyl-methanone |
| 28 | 23 | 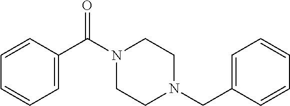 [SMILES: O=C(c1ccccc1)N1CCN(Cc2ccccc2)CC1] | (4-benzylpiperazin-1-yl)- phenyl-methanone |
| 30 | 31 | 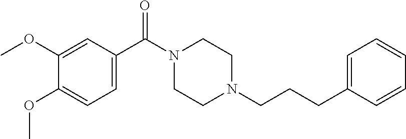 [SMILES: COc1ccc(C(=O)N2CCN(CCCc3ccccc3)CC2)cc1OC] | (3,4-Dimethoxyphenyl)-[4-(3- phenylpropyl)piperazin-1- yl]methanone |
| 32 | 35 | 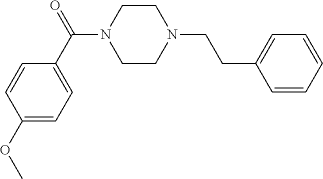 [SMILES: COc1ccc(C(=O)N2CCN(CCc3ccccc3)CC2)cc1] | (4-Methoxyphenyl)-[4-(2- phenylethyl)piperazin-1-yl] methanone |
| 33 | 38 | 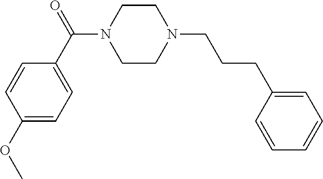 [SMILES: COc1ccc(C(=O)N2CCN(CCCc3ccccc3)CC2)cc1] | (4-Methoxyphenyl)-[4-(3- phenylpropyl)piperazin-1-yl] methanone |
| 34 | 40 | 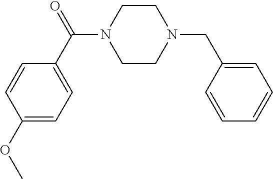 [SMILES: COc1ccc(C(=O)N2CCN(Cc3ccccc3)CC2)cc1] | (4-benzylpiperazin-1-yl)-(4- methoxyphenyl)methanone |
| 35 | 42 | 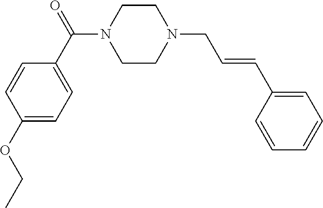 [SMILES: CCOc1ccc(C(=O)N2CCN(C/C=C/c3ccccc3)CC2)cc1] | [4-[(E)-cinnamyl]piperazin-1- yl]-(4-ethoxyphenyl)methanone |
| 36 | 43 | 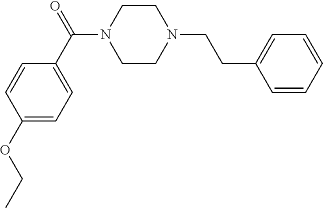 [SMILES: CCOc1ccc(C(=O)N2CCN(CCc3ccccc3)CC2)cc1] | (4-ethoxyphenyl)-[4-(2- phenylethyl)piperazin-1-yl] methanone |
| 37 | 46 | 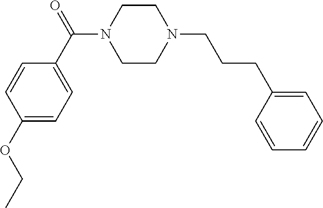 [SMILES: CCOc1ccc(C(=O)N2CCN(CCCc3ccccc3)CC2)cc1] | (4-ethoxyphenyl)-[4-(3- phenylpropyl)piperazin-1-yl] methanone |
| 38 | 48 | 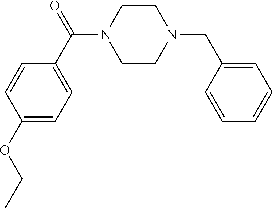 [SMILES: CCOc1ccc(C(=O)N2CCN(Cc3ccccc3)CC2)cc1] | (4-benzylpiperazin-1-yl)-(4- ethoxyphenyl)methanone |

or the active ingredient of the general formula (II) is selected from the group consisting of

TABLE 5

| Comp. | | | |
|---|---|---|---|
| No. | | Compound | Chemical name |
| 43 | 45 | 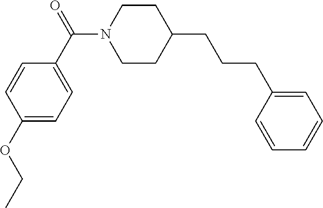 [SMILES: CCOc1ccc(C(=O)N2CCC(CCCc3ccccc3)CC2)cc1] | (4-ethoxyphenyl)-[4-(3- phenylpropyl)-1-piperidyl] methanone |

or the active ingredient of the general formula (III) is selected from the group consisting of

TABLE 6

| Comp. | | | |
|---|---|---|---|
| No. | | Compound | Chemical name |
| 44 | 22 | 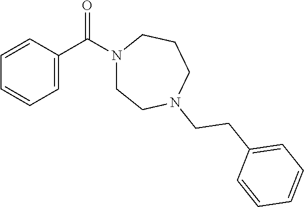 [SMILES: O=C(c1ccccc1)N1CCCN(CCc2ccccc2)CC1] | Phenyl-[4-(2-phenylethyl)-1,4- diazepan-1-yl]methanone |
| 47 | 39 | 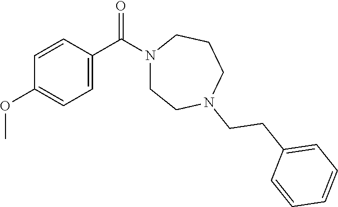 [SMILES: COc1ccc(C(=O)N2CCCN(CCc3ccccc3)CC2)cc1] | (4-Methoxyphenyl)-[4-(2- phenylethyl)-1,4-diazepan-1- yl]methanone |
| 48 | 47 | 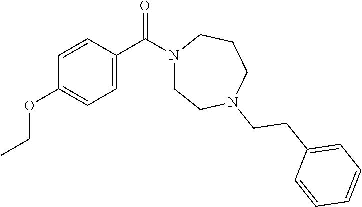 [SMILES: CCOc1ccc(C(=O)N2CCCN(CCc3ccccc3)CC2)cc1] | (4-ethoxyphenyl)-[4-(2- phenylethyl)-1,4-diazepan-1- yl]methanone |

as well as their salts, in particular acid addition salts, with inorganic or organic acids, whereby the active ingredients may be present in stereo isomerically pure form or as mixtures of different stereoisomers.

In an even more preferred variant according to the first aspect of the present invention, it is an active ingredient, wherein the active ingredient of general formula (I) is selected from the group consisting of:

TABLE 7

| Comp. | | | |
|---|---|---|---|
| No. | | Compound | Chemical name |
| 1 | 1 | 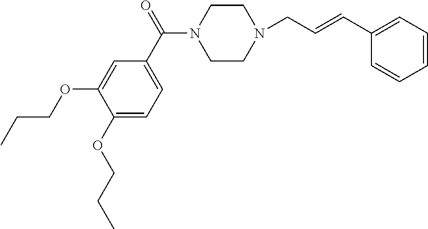 [SMILES: CCCOc1ccc(C(=O)N2CCN(C/C=C/c3ccccc3)CC2)cc1OCCC] | [4-[(E)-cinnamyl]piperazin-1- yl]-(3,4-dipropoxy- phenyl)methanone |
| 2 | 4 | 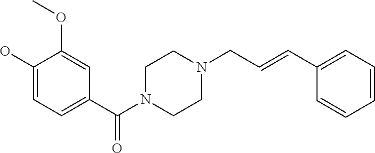 [SMILES: COc1cc(C(=O)N2CCN(C/C=C/c3ccccc3)CC2)ccc1O] | [4-[(E)-cinnamyl]piperazin-1- yl]-(4-hydroxy-3-methoxy- phenyl)methanone |
| 3 | 6 | 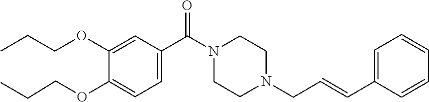 [SMILES: CCCOc1ccc(C(=O)N2CCN(C/C=C/c3ccccc3)CC2)cc1OCCC] | [4-[(E)-cinnamyl]piperazin-1- yl]-(3,4-dipropoxy- phenyl)methanone |
| 4 | 9 | 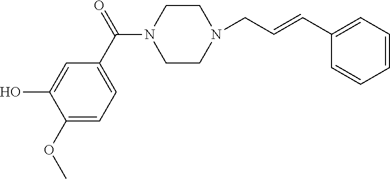 [SMILES: COc1ccc(C(=O)N2CCN(C/C=C/c3ccccc3)CC2)cc1O] | [4-[(E)-cinnamyl]piperazin-1- yl]-(3-hydroxy-4-methoxy- phenyl)methanone |
| 5 | 10 | 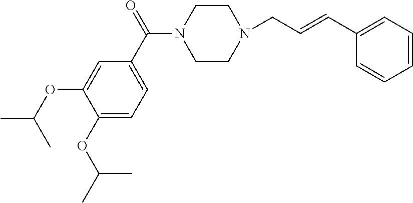 [SMILES: CC(C)Oc1ccc(C(=O)N2CCN(C/C=C/c3ccccc3)CC2)cc1OC(C)C] | [4-[(E)-cinnamyl]piperazin-1- yl]-(3,4-diisopropoxy- phenyl)methanone |
| 6 | 11 | 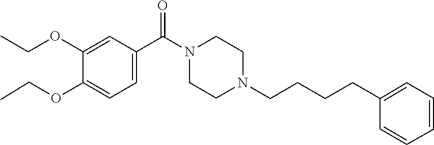 [SMILES: CCOc1ccc(C(=O)N2CCN(CCCCc3ccccc3)CC2)cc1OCC] | (3,4-Diethoxyphenyl)-[4-(4- phenylbutyl)piperazin-1- yl]methanone |
| 7 | 12 | 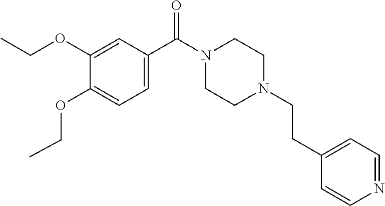 [SMILES: CCOc1ccc(C(=O)N2CCN(CCc3ccncc3)CC2)cc1OCC] | (3,4-Diethoxyphenyl)-[4-[2-(4- pyridyl)ethyl]piperazin-1- yl]methanone |
| 8 | 13 | 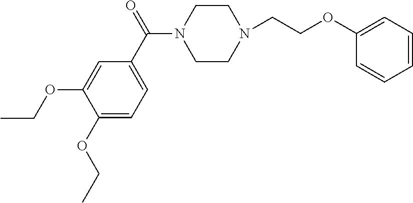 [SMILES: CCOc1ccc(C(=O)N2CCN(CCOc3ccccc3)CC2)cc1OCC] | (3,4-Diethoxyphenyl)-[4-(2- phenoxyethyl)piperazin-1- yl]methanone |
| 9 | 19 | 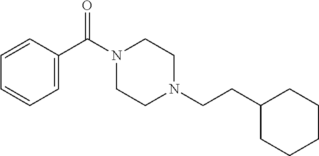 [SMILES: O=C(c1ccccc1)N1CCN(CCC2CCCCC2)CC1] | [4-(2-cyclohexylethyl)piperazin- 1-yl]-phenyl-methanone |
| 10 | 24 | 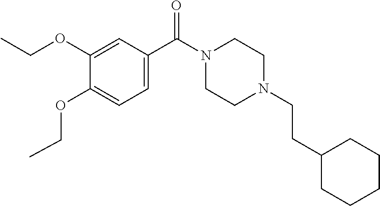 [SMILES: CCOc1ccc(C(=O)N2CCN(CCC3CCCCC3)CC2)cc1OCC] | [4-(2-cyclohexylethyl)piperazin- 1-yl]-(3,4-diethoxy- phenyl)methanone |
| 11 | 27 | 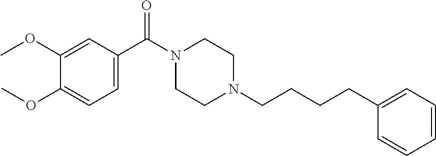 [SMILES: COc1ccc(C(=O)N2CCN(CCCCc3ccccc3)CC2)cc1OC] | (3,4-Dimethoxyphenyl)-[4-(4- phenylbutyl)piperazin-1- yl]methanone |
| 12 | 29 | 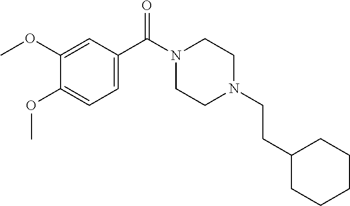 [SMILES: COc1ccc(C(=O)N2CCN(CCC3CCCCC3)CC2)cc1OC] | [4-(2-cyclohexylethyl) piperazin-1-yl]-(3,4-dimethoxy- phenyl)methanone |
| 13 | 34 | 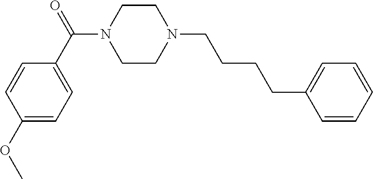 [SMILES: COc1ccc(C(=O)N2CCN(CCCCc3ccccc3)CC2)cc1] | (4-Methoxyphenyl)-[4-(4- phenylbutyl)piperazin-1-yl] methanone |
| 14 | 36 | 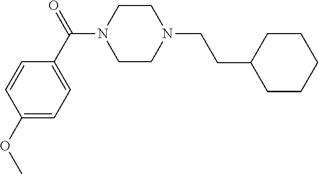 [SMILES: COc1ccc(C(=O)N2CCN(CCC3CCCCC3)CC2)cc1] | [4-(2-cyclohexylethyl) piperazin-1-yl]-(4-methoxy- phenyl)methanone |
| 15 | 41 | 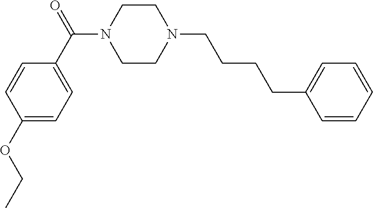 [SMILES: CCOc1ccc(C(=O)N2CCN(CCCCc3ccccc3)CC2)cc1] | (4-ethoxyphenyl)-[4-(4- phenylbutyl)piperazin-1-yl] methanone |
| 16 | 44 | 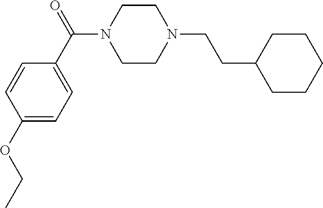 [SMILES: CCOc1ccc(C(=O)N2CCN(CCC3CCCCC3)CC2)cc1] | [4-(2-cyclohexylethyl) piperazin-1-yl]-(4-ethoxy- phenyl)methanone |

or the active ingredient of the general formula (II) is selected from the group consisting of

TABLE 8

| Comp. | | | |
|---|---|---|---|
| No. | | Compound | Chemical name |
| 40 | 25 | 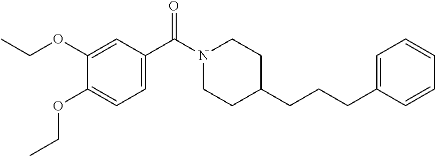 [SMILES: CCOc1ccc(C(=O)N2CCC(CCCc3ccccc3)CC2)cc1OCC] | (3,4-Diethoxyphenyl)-[4-(3- phenylpropyl)-1- piperidyl]methanone |
| 41 | 30 | 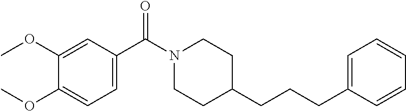 [SMILES: COc1ccc(C(=O)N2CCC(CCCc3ccccc3)CC2)cc1OC] | (3,4-Dimethoxyphenyl)-[4-(3- phenylpropyl)-1- piperidyl]methanone |
| 42 | 37 | 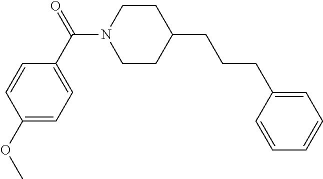 [SMILES: COc1ccc(C(=O)N2CCC(CCCc3ccccc3)CC2)cc1] | (4-Methoxyphenyl)-[4-(3- phenylpropyl)-1-piperidyl] methanone |
| 43 | 45 | 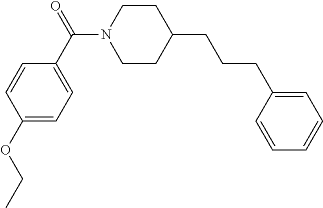 [SMILES: CCOc1ccc(C(=O)N2CCC(CCCc3ccccc3)CC2)cc1] | (4-ethoxyphenyl)-[4-(3- phenylpropyl)-1-piperidyl] methanone |

or the active ingredient of the general formula (III) is selected from the group consisting of

TABLE 9

| Comp. | | | |
|---|---|---|---|
| No. | | Compound | Chemical name |
| 45 | 26 | 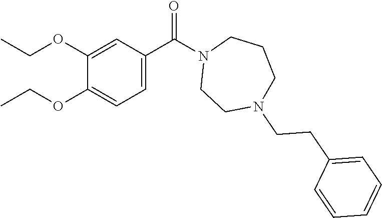 [SMILES: CCOc1ccc(C(=O)N2CCCN(CCc3ccccc3)CC2)cc1OCC] | (3,4-Diethoxyphenyl)-[4-(2- phenylethyl)-1,4-diazepan-1- yl]methanone |
| 46 | 32 | 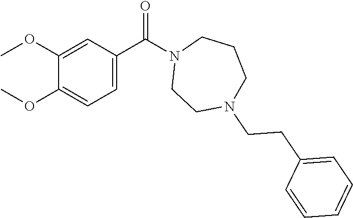 [SMILES: COc1ccc(C(=O)N2CCCN(CCc3ccccc3)CC2)cc1OC] | (3,4-Dimethoxyphenyl)-[4-(2- phenylethyl)-1,4-diazepan-1- yl]methanone |
| 47 | 39 | 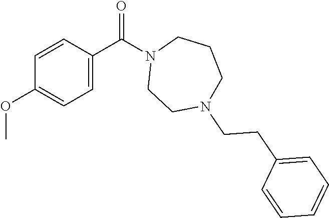 [SMILES: COc1ccc(C(=O)N2CCCN(CCc3ccccc3)CC2)cc1] | (4-Methoxyphenyl)-[4-(2- phenylethyl)-1,4-diazepan-1- yl]methanone |
| 48 | 47 | 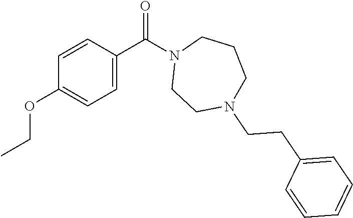 [SMILES: CCOc1ccc(C(=O)N2CCCN(CCc3ccccc3)CC2)cc1] | (4-ethoxyphenyl)-[4-(2- phenylethyl)-1,4-diazepan-1- yl]methanone |

as well as their salts, in particular acid addition salts, with inorganic or organic acids, whereby the active ingredients may be present in stereo isomerically pure form or as mixtures of different stereoisomers.

The active ingredients of the formula (I), (II) or (III) with particularly advantageous properties, i.e., with a particularly intensive trigeminal effect, i.e., with a particularly saliva-promoting and/or tingling/tingling effect, are compounds in which the active ingredient of the general formula (I) is selected from the group consisting of:

TABLE 10

| Comp. | | | |
|---|---|---|---|
| No. | | Compound | Chemical name |
| 1 | 1 | 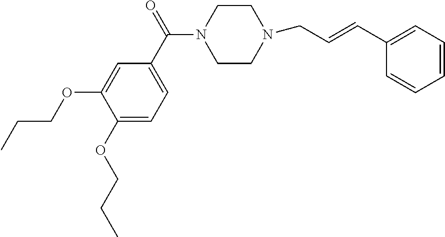 [SMILES: CCCOc1ccc(C(=O)N2CCN(C/C=C/c3ccccc3)CC2)cc1OCCC] | [4-[(E)-cinnamyl]piperazin-1- yl]-(3,4-dipropoxy- phenyl)methanone |
| 6 | 11 | 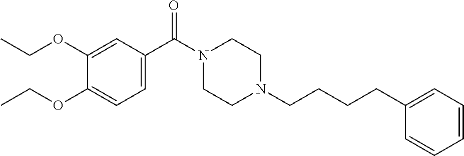 [SMILES: CCOc1ccc(C(=O)N2CCN(CCCCc3ccccc3)CC2)cc1OCC] | (3,4-Diethoxyphenyl)-[4-(4- phenylbutyl)piperazin-1- yl]methanone |
| 7 | 12 | 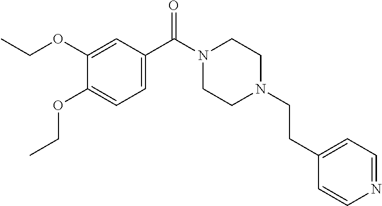 [SMILES: CCOc1ccc(C(=O)N2CCN(CCc3ccncc3)CC2)cc1OCC] | (3,4-Diethoxyphenyl)-[4-[2-(4- pyridyl)ethyl]piperazin-1- yl]methanone |
| 8 | 13 | 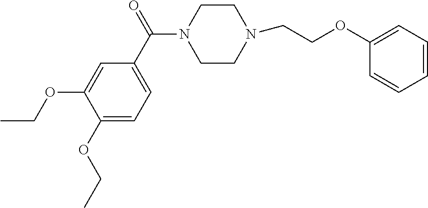 [SMILES: CCOc1ccc(C(=O)N2CCN(CCOc3ccccc3)CC2)cc1OCC] | (3,4-Diethoxyphenyl)-[4-(2- phenoxyethyl)piperazin-1- yl]methanone |
| 9 | 19 | 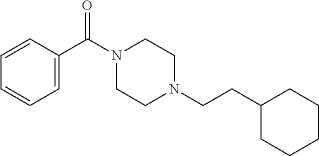 [SMILES: O=C(c1ccccc1)N1CCN(CCC2CCCCC2)CC1] | [4-(2-cyclohexylethyl)piperazin- 1-yl]-phenyl-methanone |
| 11 | 27 | 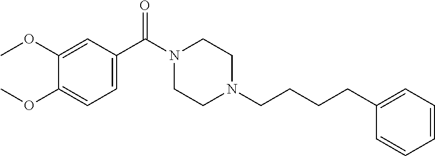 [SMILES: COc1ccc(C(=O)N2CCN(CCCCc3ccccc3)CC2)cc1OC] | (3,4-Dimethoxyphenyl)-[4-(4- phenylbutyl)piperazin-1- yl]methanone |
| 13 | 34 | 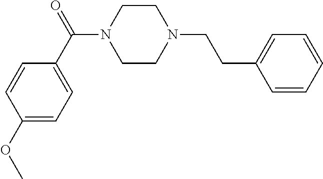 [SMILES: COc1ccc(C(=O)N2CCN(CCc3ccccc3)CC2)cc1] | (4-Methoxyphenyl)-[4-(4- phenylbutyl)piperazin-1-yl] methanone |
| 14 | 36 | 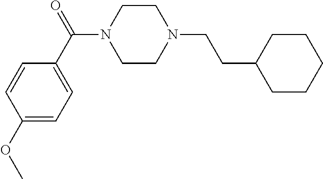 [SMILES: COc1ccc(C(=O)N2CCN(CCC3CCCCC3)CC2)cc1] | [4-(2-cyclohexylethyl) piperazin-1-yl]-(4-methoxy- phenyl)methanone |
| 15 | 41 | 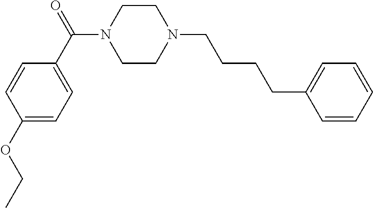 [SMILES: CCOc1ccc(C(=O)N2CCN(CCCCc3ccccc3)CC2)cc1] | (4-ethoxyphenyl)-[4-(4- phenylbutyl)piperazin-1-yl] methanone |
| 16 | 44 | 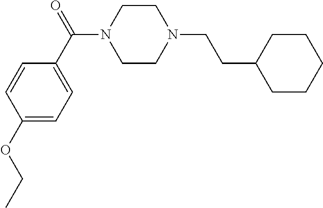 [SMILES: CCOc1ccc(C(=O)N2CCN(CCC3CCCCC3)CC2)cc1] | [4-(2-cyclohexylethyl) piperazin-1-yl]-(4-ethoxy- phenyl)methanone |

or the active ingredient of the general formula (II) is selected from the group consisting of

TABLE 11

| Comp. | | | |
|---|---|---|---|
| No. | | Compound | Chemical name |
| 43 | 45 | 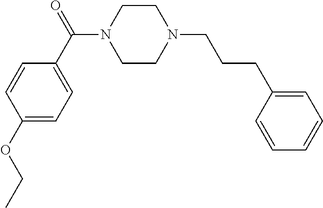 [SMILES: CCOc1ccc(C(=O)N2CCN(CCCc3ccccc3)CC2)cc1] | (4-ethoxyphenyl)-[4-(3- phenylpropyl)-1-piperidyl] methanone |

or the active ingredient of the general formula (III) is selected from the group consisting of

TABLE 12

| Comp. | | | |
|---|---|---|---|
| No. | | Compound | Chemical name |
| 47 | 39 | 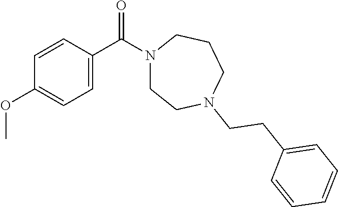 [SMILES: COc1ccc(C(=O)N2CCCN(CCc3ccccc3)CC2)cc1] | (4-Methoxyphenyl)-[4-(2- phenylethyl)-1,4-diazepan-1- yl]methanone |
| 48 | 47 | 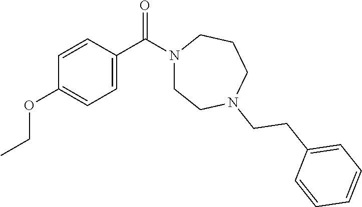 [SMILES: CCOc1ccc(C(=O)N2CCCN(CCc3ccccc3)CC2)cc1] | (4-ethoxyphenyl)-[4-(2- phenylethyl)-1,4-diazepan-1- yl]methanone |

as well as their salts, in particular acid addition salts, with inorganic or organic acids, whereby the active ingredients may be present in stereonisomericaly pure form or as mixtures of different stereoisomers.

In a most preferred embodiment according to the first aspect of the present invention, the active ingredient is selected from the group consisting of the compounds shown in Table 8 below:

TABLE 13

| Comp. | | | |
|---|---|---|---|
| No. | | Compound | Chemical name |
| 8 | 13 | 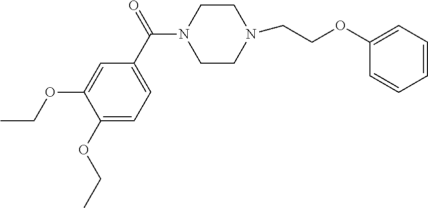 [SMILES: CCOc1ccc(C(=O)N2CCN(CCOc3ccccc3)CC2)cc1OCC] | (3,4-Diethoxyphenyl)-[4-(2- phenoxyethyl)piperazin-1- yl]methanone |
| 9 | 19 | 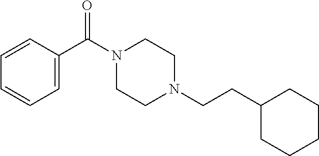 [SMILES: O=C(c1ccccc1)N1CCN(CCC2CCCCC2)CC1] | [4-(2-cyclohexylethyl)piperazin- 1-yl]-phenyl-methanone |
| 13 | 34 | 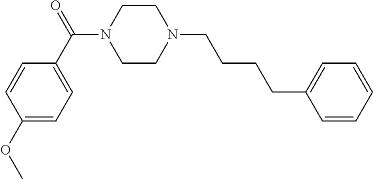 [SMILES: COc1ccc(C(=O)N2CCN(CCCCc3ccccc3)CC2)cc1] | (4-Methoxyphenyl)-[4-(4-phenyl- butyl)piperazin-1-yl]methanone |
| 15 | 41 | 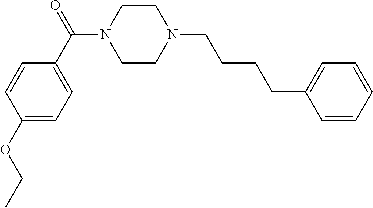 [SMILES: CCOc1ccc(C(=O)N2CCN(CCCCc3ccccc3)CC2)cc1] | (4-ethoxyphenyl)-[4-(4-phenyl- butyl)piperazin-1-yl]methanone |
| 23 | 15 | 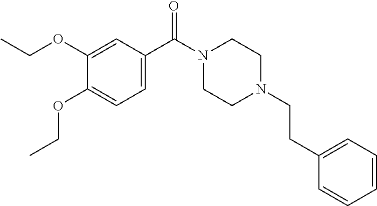 [SMILES: CCOc1ccc(C(=O)N2CCN(CCc3ccccc3)CC2)cc1OCC] | (3,4-Diethoxyphenyl)-[4-(2- phenylethyl)piperazin-1- yl]methanone |
| 36 | 43 | 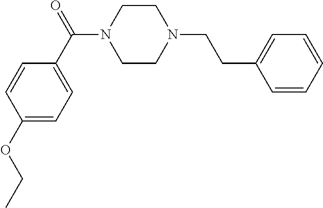 [SMILES: CCOc1ccc(C(=O)N2CCN(CCc3ccccc3)CC2)cc1] | (4-ethoxyphenyl)-[4-(2-phenyl- ethyl)piperazin-1-yl]methanone |
| 37 | 46 | 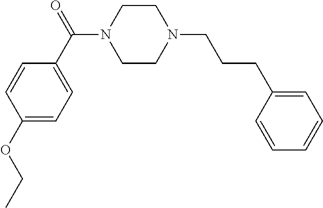 [SMILES: CCOc1ccc(C(=O)N2CCN(CCCc3ccccc3)CC2)cc1] | (4-ethoxyphenyl)-[4-(3-phenyl- propyl)piperazin-1-yl] methanone |
| 44 | 22 | 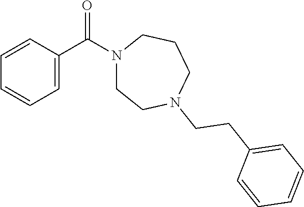 [SMILES: O=C(c1ccccc1)N1CCCN(CCc2ccccc2)CC1] | Phenyl-[4-(2-phenylethyl)-1,4- diazepan-1-yl]methanone |
| 48 | 47 | 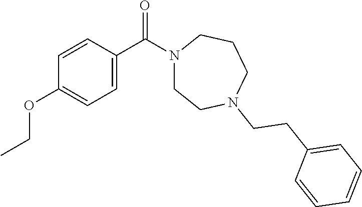 [SMILES: CCOc1ccc(C(=O)N2CCCN(CCc3ccccc3)CC2)cc1] | (4-ethoxyphenyl)-[4-(2-phenyl- ethyl)-1,4-diazepan-1- yl]methanone |

as well as their salts, in particular acid addition salts, with inorganic or organic acids, whereby the active ingredients may be present in stereoisomerically pure form or as mixtures of different stereoisomers.

The active ingredients of formula (I), (II) or (III) listed in Table 13 are characterized by a particularly pronounced trigeminal, i.e., mouth-watering and/or tingling/fizzing effect, with the mouth-watering effect being particularly pronounced. The trigeminal effect of these active ingredients occurs quickly and immediately when they are used or consumed, even in low concentrations, and lasts for a long time.

The active ingredients according to the invention can be produced by standard methods of preparative organic chemistry generally known to the skilled person. Exemplary synthesis methods are given in the following examples.

Another object of the present invention relates to a flavoring preparation which comprises or consists of:
(a) one or more active ingredient(s) according to the invention, as defined above, preferably one or more active ingredient(s) selected from the group consisting of:

TABLE 14

| Comp. | | | |
|---|---|---|---|
| No. | | Compound | Chemical name |
| 8 | 13 | 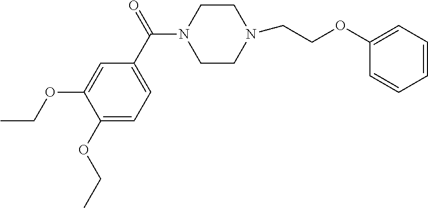 [SMILES: CCOc1ccc(C(=O)N2CCN(CCOc3ccccc3)CC2)cc1OCC] | (3,4-Diethoxyphenyl)-[4- (2-phenoxyethyl)piperazin- 1-yl]methanone |
| 9 | 19 | 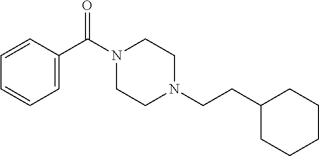 [SMILES: O=C(c1ccccc1)N1CCN(CCC2CCCCC2)CC1] | [4-(2-cyclohexylethyl) piperazin-1-yl]-phenyl- methanone |
| 13 | 34 | 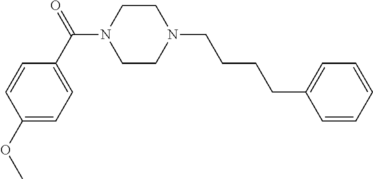 [SMILES: COc1ccc(C(=O)N2CCN(CCCCc3ccccc3)CC2)cc1] | (4-Methoxyphenyl)-[4-(4- phenylbutyl)piperazin-1- yl]methanone |
| 15 | 41 | 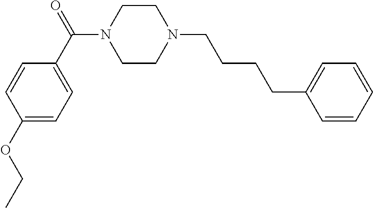 [SMILES: CCOc1ccc(C(=O)N2CCN(CCCCc3ccccc3)CC2)cc1] | (4-ethoxyphenyl)-[4-(4- phenylbutyl)piperazin-1- yl]methanone |
| 23 | 15 | 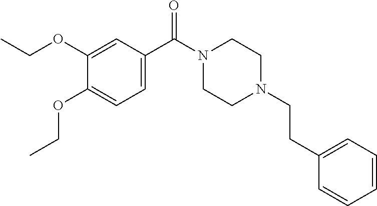 [SMILES: CCOc1ccc(C(=O)N2CCN(CCc3ccccc3)CC2)cc1OCC] | (3,4-Diethoxyphenyl)-[4- (2-phenylethyl)piperazin- 1-yl]methanone |
| 36 | 43 | 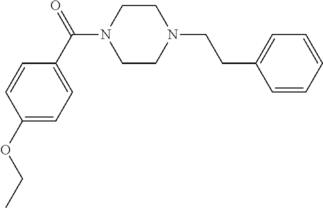 [SMILES: CCOc1ccc(C(=O)N2CCN(CCc3ccccc3)CC2)cc1] | (4-ethoxyphenyl)-[4-(2- phenylethyl)piperazin-1- yl]methanone |
| 37 | 46 | 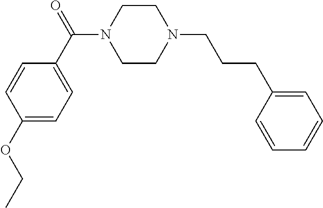 [SMILES: CCOc1ccc(C(=O)N2CCN(CCCc3ccccc3)CC2)cc1] | (4-ethoxyphenyl)-[4-(3- phenylpropyl)piperazin-1- yl]methanone |
| 44 | 22 | 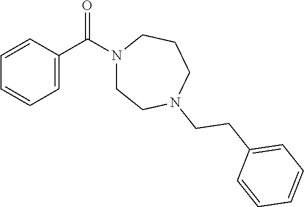 [SMILES: O=C(c1ccccc1)N1CCCN(CCc2ccccc2)CC1] | Phenyl-[4-(2-phe- nylethyl)-1,4-diazepan-1- yl]methanone |
| 48 | 47 | 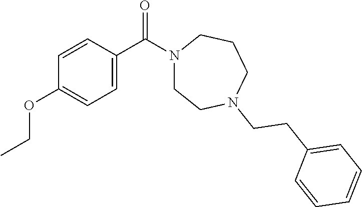 [SMILES: CCOc1ccc(C(=O)N2CCCN(CCc3ccccc3)CC2)cc1] | (4-ethoxyphenyl)-[4-(2- phenylethyl)-1,4-diazepan- 1-yl]methanone |

as well as their salts, in particular acid addition salts, with inorganic or organic acids, whereby the active ingredients may be present in stereo isomerically pure form or as mixtures of different stereoisomers.

In principle, the present invention comprises the individual active ingredients or a mixture of two or more active ingredients or compounds of the general formula (I), formula (II) and formula (III). Particularly advantageous are flavor preparations according to the invention which comprise not only one, but two, three or even several active ingredients according to the invention. The particular advantage of such flavoring preparations or mixtures of active ingredients is that a combination of several trigeminal effects such as mouth-watering and tingling or a synergistic effect can be achieved.

Nevertheless, the active ingredients or compounds according to the invention are also suitable for mixing with other, already known active ingredients, such as flavoring or aromatic substances.

In a further preferred embodiment, the present invention therefore relates to a flavoring preparation further comprising:
(b) at least one further active compound which is different from the active compound (a) of the general formula (I), formula (II) or formula (III).

With a combination of different active ingredients, different sensory, in particular gustatory, effects can be combined with each other in order to evoke different customized taste experiences, i.e., taste effects, during consumption or use.

Particularly advantageous are flavoring preparations according to the invention which comprise combinations of one or more active ingredient(s) according to the invention of the general formula (I), formula (II) or formula (III) which forms component (a), with one or more further active ingredient(s) which forms component (b) and is/are different from the active ingredient(s) according to the invention. Such a combination allows, for example, different sensory effects to be combined with each other, such as mouth-watering and warming effect, mouth-watering and cooling effect, etc., or by using them together another effect, such as a cooling effect, can be enhanced, or another effect, such as an astringent effect, can be weakened.

Suitable further active ingredients which form component (b) and are different from the active ingredient(s) according to the invention which form(s) component (a) are selected from the group consisting of substances which produce a sharp taste or a sensation of warmth or heat on the skin or mucous membranes or a tingling or fizzing sensation in the mouth or throat, substances with an astringent effect and substances with a physiological cooling effect.

The heat-inducing or pungent active ingredients are preferably selected from the group consisting of: Paprika powder, chili pepper powder, extracts from paprika, extracts from pepper, extracts from chili pepper, extracts from ginger roots, extracts from grains of paradise (Aframomum melegueta), extracts from paracress (Jambu oleoresin; Spilanthes acmella, resp. Spilanthes oleracea), extracts from Japanese pepper (Zanthoxylum piperitum), extracts from Kaempferia galanga, extracts from Alpinia galanga, extracts from water pepper (Polygonium hydropiper), capsaicinoids, in particular capsaicin, dihydrocapsaicin or nonivamide; Gingerols, in particular gingerol-[6], gingerol-[8], or gingerol-[10]; shogaols, in particular shogaol-[6], shogaol-[8], shogaol-[10]; gingerdiones, in particular gingerdione-[6], gingerdione-[8] or gingerdione-[10]; paradoles, in particular paradol-[6], paradol-[8] or paradol-[10]; dehydrogingerdiones, in particular dehydrogingerdione-[6], dehydrogingerdione-[8] or dehydrogingerdione-[10]; piperine; piperine derivatives; ethyl 2-(4-hydroxy-3-methoxyphenyl)acetate and 3-phenylpropyl-2-(4-hydroxy-3-methoxy-phenyl)acetate and mixtures thereof.

The active ingredients which can be perceived as pungent or acrid are preferably selected from the group consisting of: aromatic isothiocyanates, in particular phenylethyl isothiocyanate, allyl isothiocyanate, cyclopropyl isothiocyanate, butyl isothiocyanate, 3-methylthiopropyl isothiocyanate, 4-hydroxybenzyl isothiocyanate, 4-methoxybenzyl isothiocyanate and mixtures thereof.

The tingling agents are preferably selected from the group consisting of 2E,4E-decadienoic acid-N-isobutylamide (trans-pellitorin), in particular those as described in WO 2004/043906; 2E,4Z-decadienoic acid-N-isobutylamide (cis-pellitorin), in particular those as described in WO 2004/000787; 2Z,4Z-decadienoic acid-N-isobutylamide; 2Z,4E-decadienoic acid-N-isobutylamide; 2E,4E-decadienoic acid-N-([2S]-2-methylbutyl)amide; 2E,4E-decadienoic acid-N-([2S]-2-methylbutyl)amide; 2E,4E-decadienoic acid-N-([2R]-2-methylbutylamide); 2E,4Z-decadienoic acid-N-(2-methylbutyl)amide; 2E,4E-decadienoic acid-N-piperide (achilleamide); 2E,4E-decadienoic acid-N-piperide (sarmentin); 2E-decenoic acid-N-isobutylamide; 3E-decenoic acid-N-isobutylamide; 3E-nonenoic acid-N-isobutylamide; 2E,6Z,8E-decatrienoic acid-N-isobutylamide (spilanthol); 2E,6Z,8E-decatrienoic acid-N-([2S]-2-methylbutyl)amide (homospilanthol); 2E,6Z,8E-decatrienoic acid-N-([2R]-2-methylbutyl)amide; 2E-decen-4-ynoic acid-N-isobutylamide; 2Z-decen-4-ynoic acid-N-isobutylamide; 2E,6Z,8E,10E-dodecatetraenoic acid-N-(2-methylpropyl)amide (alpha-sanshool); 2E,6Z,8E,10E-dodecatetraenoic acid-N-(2-hydroxy-2-methylpropyl)amide (alpha-hydroxysanshool); 2E,6E,8E,10E-dodecatetraenoic acid-N-(2-hydroxy-2-methylpropyl)amide (gamma-hydroxysanshool); 2E,4E,8Z,10E,12E-tetradecapentaenoic acid-N-(2-hydroxy-2-methylpropyl)amide (gamma-hydroxysanshool); 2E,4E,8E,10E,12E-tetradecapentaenoic acid-N-(2-hydroxy-2-methylpropyl)-amide (gamma-hydroxyisosanshool); 2E,4E,8Z,10E,12E-tetradecapentaenoic acid-N-(2-methyl-2-propenyl)amide (gamma-dehydrosanshool); 2E,4E,8Z,10E,12E-tetradecapentaenoic acid-N-(2-methylpropyl)amide (gamma-sanshool); 2E,4E,8Z,11Z-Tetradecatetraenoic acid-N-(2-hydroxy-2-methylpropyl)amide (bungeanool); 2E,4E,8Z,11E-tetradecatetraenoic acid-N-(2-hydroxy-2-methylpropyl)amide (isobungeanool); 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)amide (tetrahydrobungeanool) and mixtures thereof.

Active ingredients with an astringent effect are preferably selected from the group consisting of: Catechins, in particular epicatechins, gallocatechins, epigallocatechins as well as their respective gallic acid esters, in particular epigallocatechin gallate or epicatechin gallate, their oligomers (procyanidins, proanthocyanidins, prodelphinidins, procyanirins, thearubigenins, theogallins) as well as their C- and O-glycosides; Dihydroflavonoids such as dihydromyricetin, taxifolin, and their C- and O-glycosides, flavonols such as myricetin, quercetin and their C- and O-glycosides such as quercetrin, rutin, gallic acid esters of carbohydrates such as tannin, pentagalloyl glucose or their reaction products such as elligatannin, aluminum salts, e.g. alum, and their reaction products such as elligatannin. e.g. alum, and their mixtures.

The active ingredients with a physiological cooling effect or the so-called cooling agents are substances that produce a cooling sensory effect on the skin or mucous membranes when consumed or applied, although no physical cooling, such as evaporation, actually takes place. Such cooling agents are preferably selected from the group consisting of: Substances with physiological cooling effect, selected from group consisting of: Menthol, Menthol Methyl Ether (FEMA GRAS 4054), Monomenthyl Glutamate (FEMA GRAS 4006), Menthoxy-1,2-propanediol (FEMA GRAS 3784), Dimenthyl Glutarate (FEMA GRAS 4604), Hydroxymethylcyclohexylethanone (FEMA GRAS 4742), 2-(4-ethylphenoxy)-N-(1H-pyrazol-3-yl)-N-(thiophen-2-ylmethyl)acetamide (FEMA GRAS 4880), WS-23 (2-isopropyl-N,2,3-trimethylbutyramide (FEMA GRAS 3804), N-(4-(cyanomethyl)phenyl)-2-isopropyl-5,5-dimethylcyclohexane carboxamide (FEMA GRAS 4882), N-(3-hydroxy-4-methoxyphenyl)-2-isopropyl-5,5-dimethylcyclohexane carboxamide (FEMA GRAS 4881), N-(2-hydroxy-2-phenylethyl)-2-isopropyl-5,5-dimethylcyclohexane-1-carboxamide (FEMA GRAS 4896), 3,4-methylenedioxy cinnamic acid, (E)-3-benzo[1,3]dioxol-5-yl-N,N-diphenyl-2-propenamide (FEMA GRAS 4788), menthol propylene glycol carbonate (FEMA GRAS 3806), menthyl N-ethyloxamate, monomethyl succinate (FEMA GRAS 3810), WS-3 (N-ethyl-p-menthane-3-carboxamide, FEMA GRAS 3455), menthol ethylene glycol carbonate (FEMA GRAS 3805), WS-5 (ethyl 3-(p-menthane-3-carboxamido)acetate, FEMA GRAS 4309), WS-12 (1R,2S,5R)—N-(4-methoxyphenyl)-p-menthanecarboxamide (FEMA GRAS 4681), WS-27 (N-ethyl-2,2-diisopropylbutanamide, FEMA GRAS 4557), N-cyclopropyl-5-methyl-2-isopropylcyclohexanecarboxamide (FEMA GRAS 4693), WS-116 (N-(1,1-dimethyl-2-hydroxyethyl)-2,2-diethylbutanamide, FEMA GRAS 4603), menthoxyethanol (FEMA GRAS 4154), N-(4-cyanomethylphenyl)-p-menthanecarboxamide (FEMA GRAS 4496), N-(2-(pyridin-2-yl)ethyl)-3-p-menthanecarboxamide (FEMA GRAS 4549), N-(2-hydroxyethyl)-2-isopropy-1-2,3-dimethylbutanamide (FEMA GRAS 4602), (2S,5R)—N-[4-(2-amino-2-oxoethyl)phenyl]-p-menthanecarboxamide (FEMA GRAS 4684), N-cyclopropyl-5-methyl-2-isopropylcyclohexanecarboxamide (FEMA GRAS 4693), 2-[(2-p-menthoxy)ethoxy]-ethanol (FEMA GRAS 4718), (2,6-diethyl-5-isopropyl-2-methyltetrahydropyran (FEMA GRAS 4680), trans-4-tert-butylcyclohexanol (FEMA GRAS 4724), 2-(p-Tolyloxy)-N-(1H-pyrazol-5-yl)-N-((thiophen-2-yl)methyl)acetamide (FEMA GRAS 4809), menthone glycerol ketal (FEMA GRAS 3807 and 3808), (−)-menthoxypropane-1,2-diol, 3-(1-menthoxy)-2-methylpropane-1,2-diol (FEMA GRAS 3849), isopulegol, (+)-cis and (−)-trans-p-menthane-3,8-diol (62:38, FEMA GRAS 4053), 2,3-dihydroxy-p-menthane, 3,3,5-trimethylcyclohexanone glycerol ketal, menthyl pyrrolidone carboxylate, (1R,3R,4S)-3-menthyl-3,6-dioxaheptanoate, (1R,2S,5R)-3-menthyl methoxyacetate, (1R,2S,5R)-3-menthyl-3,6,9-trioxadecanoate, (1R,2S,5R)-3-menthyl-3,6,9-trioxadecanoate, (1R,2S,5R)-3-menthyl-(2-hydroxyethoxy)acetate, (1R,2S,5R)-menthyl-1,1-hydroxy-3,6,9-trioxaundecanoate, cubebol (FEMA GRAS 4497), 2-isopropyl-5-methylcyclohexyl-4-(dimethylamino)-4-oxobutanoate (FEMA GRAS 4230), menthyl lactate (FEMA GRAS 3748), 6-isopropyl-3,9-dimethyl-1,4-dioxaspiro[4.5]decan-2-one (FEMA GRAS 4285), N-benzo[1,3]-dioxol-5-yl-3-p-menthanecarboxamide, N-(1-isopropyl-1,2-dimethylpropyl)-1,3-benzodioxole-5-carboxamide, N—(R)-2-oxotetrahydrofuran-3-yl-(1R,2S,5R)-p-menthane-3-carboxamide, mixture of 2,2,5,6,6-pentamethyl-2,3,6,6a-tetrahydropentalene-3a(1H)-ol and 5-(2-hydroxy-2-methylpropyl)-3,4,4-trimethylcyclopent-2-en-1-one; (2S,5R)-2-isopropyl-5-methyl-N-(2-(pyridin-4-yl)ethyl)cyclohexanecarboxamides; (1 S,2S,5R)—N-(4-(cyanomethyl)phenyl)-2-isopropyl-5-methylcyclohexanecarboxamide, 1,7-isopropyl-4,5-methyl-bicyclo[2.2.2]oct-5-ene derivatives, 4-methoxy-N-phenyl-N-[2-(pyridin-2-yl)ethyl]benzamide, 4-methoxy-N-phenyl-N-[2-(pyridin-2-yl)ethyl]benzenesulfonamide, 4-chloro-N-phenyl-N-[2-(pyridin-2-yl)ethyl]benzenesulfonamide, 4-cyano-N-phenyl-N-[2-(pyridin-2-yl)ethyl]-benzenesulfonamide, 4-((benzhydrylamino)methyl)-2-methoxyphenol, 4-((bis(4-methoxyphenyl)methylamino)methyl)-2-methoxyphenol, 4-((1,2-diphenylethylamino)methyl)-2-methoxyphenol, 4-((benzhydryloxy)methyl)-2-methoxyphenol, 4-((9H-fluoren-9-ylamino)methyl)-2-methoxyphenol, 4-((benzhydrylamino)methyl)-2-ethoxyphenol, 1-(4-methoxyphenyl)-2-(1-methyl-1H-benzo[d]imidazol-2-yl)vinyl-4-methoxybenzoate, 2-(1-isopropyl-6-methyl-1H-enzo[d]imidazol-2-yl)-1-(4-methoxyphenyl)vinyl-4-methoxybenzoate, (Z)-2-(1-isopropyl-5-methyl-1H-benzo[d]imidazol-2-yl)-1-(4-methoxyphenyl)vinyl-4-methoxybenzoate, 3-alkyl-p-methan-3-ol derivatives, derivatives of fenchyl, D-bornyl, L-bornyl, exo-norbornyl, 2-methylisobornyl, 2-ethylfenchyl, 2-methylbornyl, cis-pinan-2-yl, verbanyl and isobornyl, menthyloxamate derivatives, menthyl 3-oxocarboxylic acid esters, N-alpha-(menthanecarbonyl)amino acid amides, p-menthane carboxamide and WS-23 analogs, (−)(1R,2R,4S)-dihydroumbellulol, p-menthane alkyloxyamide, cyclohexane derivatives, butanone derivatives, mixture of 3-menthoxy-1-propanol and 1-menthoxy-2-propanol, 1-[2-ydroxyphenyl]-4-[2-nitrophenyl]-1,2,3,6-tetrahydropyrimidin-2-one, 4-methyl-3-(1-pyrrolidinyl)-2-[5H]-furanone and mixtures thereof.

FEMA stands for “Flavor and Extracts Manufacturers Association” and GRAS is defined as “Generally Regarded As Safe”. A FEMA GRAS designation means that the labeled substance has been tested according to standard methods and is considered toxicologically safe. In principle, all known substances with a cooling effect are suitable as component (b). For reasons of food safety, however, those compounds with a FEMA GRAS designation are preferred.

The semi-finished flavor preparations according to the invention preferably contain component (a) or alternatively component (a) plus component (b) in an amount of from 0.1% by weight to 20% by weight, preferably from 1% by weight to 15% by weight, even more preferably from 2% by weight to 10% by weight, most preferably from 3% by weight to 5% by weight.

A flavor preparation as a semi-finished product in the context of the present invention is such a product that is later processed into a finished product or such a product that is later incorporated into another product to produce a ready-to-use product or final formulation.

The flavoring preparations according to the invention may contain the components (a) and (b) in a weight ratio of about 0.1:99.9 to about 99.0:0.1, preferably about 1:99 to about 99:1, even more preferably 10:90 to about 90:10, still more preferably about 25:75 to about 75:25 and in particular about 40:60 to 60:40.

Such a composition of the flavoring preparation according to the invention is particularly advantageous, as it allows the amount of active ingredient(s) in the final formulation or the finished product to be controlled.

In a further preferred variant, the flavoring preparation according to the invention further comprises
(c) at least one aroma or flavoring agent which is different from the active ingredient (a) according to general formula (I), general formula (II) or general formula (III) and the active ingredient (b).

Such combinations can be used to create new aroma or flavor compositions in which olfactory and/or gustatory (sweet, sour, salty, bitter and umami) effects are combined or supplemented with trigeminal effects (mouth-watering, tingling) in an advantageous way.

Preferred is a flavoring preparation according to the invention in which the at least one aroma or flavoring substance which is different from the active ingredient (a) and the active ingredient (b) according to the invention has an unpleasant, in particular bitter taste quality, or an astringent, dry, dusty, floury, chalky and/or metallic note.

The particular advantage of such mixtures or flavoring preparations is that the active ingredients according to the invention are capable of correcting, i.e., reducing or masking or neutralizing unpleasant taste impressions of flavors, for example the bitterness of sweeteners, even in small concentrations.

The one or more flavoring agents contained in the flavoring preparation according to the invention is/are selected from the group consisting of: Acetophenone, allyl capronate, alpha-ionone, beta-ionone, anisaldehyde, anisyl acetate, anisyl formate, anethole, benzaldehyde, benzothiazole, benzyl acetate, benzyl alcohol, benzyl benzoate, beta-ionone, butyl butyrate, butyl capronate, butylidene phthalide, carvone, camphene, caryophyllene, cineole, cinnamyl acetate, citral, citronellol, citronellal, citronellyl acetate, cyclohexyl acetate, cymene, damascone, decalactone, dihydrocoumarin, dimethyl anthranilate, dimethyl anthranilate, dodecalactone, ethoxyethyl acetate, ethyl butyric acid, ethyl butyrate, ethyl caprinate, ethyl capronate, ethyl crotonate, ethyl furaneol, ethyl guaiacol, ethyl isobutyrate, ethyl isovalerate, ethyl lactate, ethyl methyl butyrate, ethyl propionate, eucalyptol, eugenol, ethyl heptylate, 4-(p-hydroxyphenyl)-2-butanone, gamma-decalactone, geraniol, geranyl acetate, geranyl acetate, grapefruit aldehyde, methyldihydrojasmonate (e.g. B. Hedion®), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenyl acetate, cis-3-hexenyl acetate, cis-3-hexenyl capronate, trans-2-hexenyl capronate, cis-3-hexenyl formate, cis-2-hexyl acetate, cis-3-hexyl acetate, trans-2-hexyl acetate, cis-3-hexyl formate, para-hydroxybenzylacetone, isoamyl alcohol, isoamyl isovalerate, isobutyl butyrate, isobutyraldehyde, isoeugenol methyl ether, isopropyl methyl thiazole, lauric acid, leavulinic acid, linalool, linalool oxide, linalyl acetate, menthol, menthofuran, methyl anthranilate, methyl butanol, methyl butyric acid, 2-methyl butyl acetate, methyl capronate, methyl cinnamate, 5-methyl furfural, 3,2,2-methyl cyclopentenolone, 6,5,2-methyl heptenone, methyl dihydrojasmonate, methyl jasmonate, 2-methyl methyl butyrate, 2-methyl-2-pentenolic acid, methyl thiobutyrate, 3,1-methyl thiohexanol, 3-methyl thiohexyl acetate, nerol, neryl acetate, trans,trans-2,4-nonadienal, 2,4-nonadienol, 2,6-nonadienol, 2,4-nonadienol, nootkatone, delta octalactone, gamma octalactone, 2-octanol, 3-octanol, 1,3-octenol, 1-octyl acetate, 3-octyl acetate, palmitic acid, paraldehyde, phellandrene, pentanedione, phenylethyl acetate, phenylethyl alcohol, phenylethyl alcohol, phenylethyl isovalerate, piperonal, propionaldehyde, propyl butyrate, pulegone, pulegol, sinensal, sulfurol, terpinene, terpineol, terpinolene, 8,3-thiomenthanone, 4,4,2-thiomethylpentanone, thymol, delta-undecalactone, gammaundecalactone, valencene, valeric acid, vanillic acid, vanillin, acetoin, ethyl vanillin, ethyl vanillin isobutyrate (=3-ethoxy-4-isobutyryloxybenzaldehyde), 2,5-dimethyl-4-hydroxy-3(2H)furanone and its derivatives (preferably homofuraneol (=2-ethyl-4-hydroxy-5-methyl-3(2H)-furanone), homofuronol (=2-ethyl-5-methyl-4-hydroxy-3(2H)-furanone and 5-ethyl-2-methyl-4-hydroxy-3(2H)-furanone), maltol and maltol derivatives (preferably ethylmaltol), coumarin and coumarin derivatives, gamma-lactones (preferably gamma-undecalactone, gamma-nonalactone, gamma-decalactone), delta-lactones (preferably 4-methyldeltadecalactone, massoilactone, deltadecalactone, tuberolactone), methyl sorbate, divanillin, 4-hydroxy-2(or 5)-ethyl-5(or 2)-methyl-3(2H)furanone, 2-hydroxy-3-methyl-2-cyclopentenone, 3-hydroxy-4,5-dimethyl-2(5H)-furanone, acetic acid isoamyl ester, butyric acid ethyl ester, butyric acid n-butyl ester, butyric acid isoamyl ester, 3-methyl-butyric acid ethyl ester, n-hexanoic acid ethyl ester, n-hexanoic acid allyl ester, n-hexanoic acid n-butyl ester, n-octanoic acid ethyl ester, ethyl 3-methyl-3-phenylglycidate, ethyl 2-trans-4-cis-decadienoate, 4-(p-hydroxyphenyl)-2-butanone, 1,1-dimethoxy-2,2,5-trimethyl-4-hexane, 2,6-dimethyl-5-hepten-1-al and phenylacetaldehyde, 2-methyl-3-(methylthio)furan, 2-methyl-3-furanethiol, bis(2-methyl-3-furyl)disulfide, furfuryl mercaptan, methional, 2-acetyl-2-thiazoline, 3-mercapto-2-pentanone, 2,5-dimethyl-3-furanethiol, 2,4,5-trimethylthiazole, 2-acetylthiazole, 2,4-dimethyl-5-ethylthiazole, 2-acetyl-1-pyrroline, 2-methyl-3-ethylpyrazine, 2-ethyl-3,5-dimethylpyrazine, 2-ethyl-3,6-dimethylpyrazine, 2,3-diethyl-5-methylpyrazine, 3-isopropyl-2-methoxypyrazine, 3-isobutyl-2-methoxypyrazine, 2-acetylpyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-penten-3-one, 4-hydroxy-2,5-dimethyl-3(2H)-furanone, guaiacol, 3-hydroxy-4,5-dimethyl-2(5H)-furanone, 3-hydroxy-4-methyl-5-ethyl-2(5H)-furanone, cinnamaldehyde, cinnamic alcohol, methyl salicylate, isopulegol and stereoisomers, enantiomers, positional isomers, diastereomers, cis/trans isomers or epimers (not explicitly mentioned here) of these substances; and/or epimers of these substances; and/or wherein the flavoring agent forming component (c) is selected from the group consisting of: Erythritol, Threitol, Arabitol, Ribotol, Xylitol, Sorbitol, Mannitol, Dulcitol, Lactitol, Miraculin, Monellin, Thaumatin, Curculin, Brazzein, Magap, Sodium cyclamate, acesulfame K, neohesperidin dihydrochalcone, saccharin sodium salt, aspartame, superaspartame, neotame, alitame, sucralose, stevioside, rebaudioside, Lugduname, Carrelame, Sucrononate, Sucrooctate, Monatine, Phenylodulcin, Glycine, D-Leucine, D-Threonine, D-Asparagine, D-Phenylalanine, D-tryptophn, L-Proline, Hernandulcin, Dihydrochalconglycosides, Glycyrrhizin, Glycerrhetinic acid, its derivatives and salts, extracts of licorice (Glycyrrhizza glabra ssp.), Lippia dulcis extracts, Momordica ssp. extracts, mogrosides, Hydrangea dulcis and steviosides and mixtures thereof. In the case of chiral compounds, the flavoring substances may be present as a racemate or as a single enantiomer or as an enantiomerically enriched mixture.

Other suitable flavoring substances include complex natural raw materials such as extracts and essential oils obtained from plants, or fractions and uniform substances obtained from these, as well as uniform synthetic or biotechnologically obtained flavoring substances.

Examples of natural raw materials are peppermint oils, spearmint oils, Mentha arvensis oils, anise oils, clove oils, citrus oils, cinnamon bark oils, wintergreen oils, cassia oils, davana oils, spruce needle oils, eucalyptus oils, fennel oils, galbanum oils, ginger oils, chamomile oils, caraway oils, rose oils, geranium oils, sage oils, parsley oils, sheaf oils, star anise oils, thyme oils, juniper berry oils, rosemary oils, angelica root oils, and the fractions of these oils.

Preferably, the aroma or flavoring agent having an unpleasant taste quality, in particular an astringent, bitter, dry, dusty, floury, chalky and/or metallic note, is selected from the group consisting of: Xanthine alkaloids, xanthines (caffeine, theobromine, theophylline and methylxanthines), alkaloids (quinine, brucine, strychnine, nicotine), phenolic glycosides (e.g. salicin, arbutin), flavonoid glycosides (e.g. neohesperidin, hesperidin, naringin, quercitrin, rutin, hyperoside, quercetin-3-O-glucoside, myricetin-3-O-glycosides), chalcones or chalcone glycosides (e.g. phloridzin, phloridzin xylosides), hydrolyzable tannins (gallic or elagic acid esters of carbohydrates, e.g. pentagalloyl glucose, tannic acids), non-hydrolyzable tannins (possibly galloylated catechins, gallocatechins, epigallocatechins or epicatechins and their oligomers, e.g. proanthyocyanidins or procyanidins, thearubigenin), flavones (e.g. quercetin, taxifolin, myricetin), phenols such as salicin, polyphenols (e.g. gamma-oryzanol, caffeic acid or its esters (e.g. chlorogenic acid and isomers)), terpenoid bitter and tannins (e.g. Limonoide wie Limonin oder Nomilin aus Zitrusfruchten, Lupolone und Humolone aus Hopfen, Iridoide, Secoiridoide), Absinthin aus Wermut, Amarogentin aus Enzian, metallische Salze (insbesondere Kalium-, Magnesium- und Calciumsalze, Kaliumchlorid, Kaliumgluconat, potassium carbonate, potassium sulphate, potassium lactate, potassium glutamate, potassium succinate, potassium malate, sodium sulphate, magnesium sulphate, aluminum salts, zinc salts, tin salts, iron (II) salts, iron (III) salts, chromium (II) picolinate), active pharmaceutical ingredients (eg. B. fluoroquinolone antibiotics, paracetamol, aspirin, beta-lactam antibiotics, ambroxol, propylthiouracil [PROP], guaifenesin), vitamins (e.g. vitamin H, B-series vitamins such as vitamin B1, B2, B6, B12, niacin, Panthotenssure), Denatoniumbenzoat, Sucraloseoctaacetat, Eisensalze, Aluminiumsalze, Zinksalze, Harnstoff, ungessttigte Fettssuren, insbesondere ungessttigte Fettssuren in Emulsionen, bitter/adstringierend schmeckende Aminossuren (z.e.g. leucine, isoleucine, valine, tryptophan, proline, histidine, tyrosine, lysine or phenylalanine) and bitter or astringent tasting peptides or proteins (in particular peptides with an amino acid from the group leucine, isoleucine, valine, tryptophan, proline or phenylalanine at the N- or C-terminus), saponins, in particular soy saponins, isotropic saponins, isotropic saponins and isotropic saponins. Soy saponins, isoflavonoids (esp. genistein, daidzein, genistin, daidzin, their glycosides and acylated glycosides); as well as substances from the group of sweeteners or sugar substitutes, preferably aspartame, acesulfame K, neotame, superaspartame, saccharin, sucralose, tagatose, monellin, steviosides, rebaudiosides, rebaudioside A, rebaudioside B, rebaudioside C, rebaudioside D, rebaudioside X, rubusosides, hernandulcin, thaumatin, miraculin, glycyrrhizin, glycyrrhetinic acid, balansin A or balansin B, or derivatives thereof, cyclamate or the pharmaceutically acceptable salts of the aforementioned compounds.

For the purposes of the present invention, artificial as well as natural sweeteners and sweetener enhancers may also be considered as aroma or flavoring agents. These can be selected from the group consisting of
Sugar alcohols (e.g., erythritol, threitol, arabitol, ribotol, xylitol, sorbitol, mannitol, dulcitol, lactitol);
proteins (e.g., miraculin, monellin, thaumatin, curculin, brazzein);
Synthetic sweeteners (e.g., Magap, sodium cyclamate, acesulfame K, neohesperidin dihydrochalcone, saccharin sodium salt, aspartame, superaspartame, neotame, alitame, sucralose, stevioside, rebaudioside, lugduname, carrelame, sucrononate, sucrooctate, monatine, phenylodulcin);
Sweet-tasting amino acids (e.g. glycine, D-leucine, D-threonine, D-asparagine, D-phenylalanine, D-tryptophn, L-proline);
Sweet-tasting low-molecular substances, such as hernandulcin, dihydrochalcone glycosides, glycyrrhizin, glycerrhetinic acid, its derivatives and salts, extracts of licorice (Glycyrrhizza glabra ssp.), Lippia dulcis extracts, Momordica ssp. extracts or
plant extracts such as Momordica grosvenori [Luo Han Guo] and the mogrosides obtained from it, Hydrangea dulcis or Stevia ssp. (e.g. Stevia rebaudiana) extracts or steviosides obtained from it.

The active ingredients or flavoring preparations according to the invention can be used in pure form, as solutions or also in specially prepared form and incorporated into ready-to-use products or final formulations.

Preferably, the active ingredient or the flavoring preparation according to the invention is present in the form of a solution and therefore comprises, on the other hand, at least one solvent as a further component.

Individual solvents or solvent systems have proven to be advantageous, wherein the solvent is selected from the group consisting of benzyl alcohol, 2-phenylethanol, benzyl benzoate, diethyl succinate, triethyl citrate, triacetin, ethanol, peppermint oil, anethole, optamint, propylene glycol and mixtures thereof.

The solvent is preferably present in an amount of from 80% to 99% by weight, preferably from 85% to 99% by weight, even more preferably from 90% to 99% by weight, most preferably from 95% to 99% by weight, based on the total weight of the active ingredient preparation or flavoring preparation, with the proviso that components (a) and/or (b) and the solvent together make up 100% by weight.

The active ingredients or flavoring preparations according to the invention can be bound to a carrier, spray-dried or encapsulated. In the bound form, compounds according to the invention can be bound to or in a carrier, e.g., common salt, sugar, starches or sugar melts. The spray-dried form is prepared from the liquid compositions by preparing an emulsion with the addition of certain amounts of a carrier, preferably biopolymers such as starch, modified starches, maltodextrin and gum arabic. This emulsion is dried in spray dryers by finely dispersing it while simultaneously applying a temperature. The result is a powder with the desired load of liquid composition.

In a further preferred variant of the invention, the active ingredient or the flavoring preparation according to the invention is present in encapsulated form.

Capsules are spherical aggregates containing at least one solid or liquid core enclosed in at least one continuous shell. During encapsulation, the one or more refrigerant(s), or the refrigerant mixture or the flavoring preparation is encapsulated with the aid of a coating material/envelope material and is present as macrocapsules with diameters of about 0.1 to about 5 mm or microcapsules with diameters of about 0.0001 to about 0.1 mm.

In these capsules, the liquid or solid active ingredients flavor preparations according to the invention are enclosed and can be released by various mechanisms such as heat application, pH shift or chewing pressure.

Suitable coating materials are, for example, starches, including their degradation products and chemically or physically produced derivatives (in particular dextrins and maltodextrins), gelatine, gum arabic, agar-agar, ghatti gum, gellan gum, modified and unmodified celluloses, pullulan, curdlan, carrageenans, alginic acid, alginates, pectin, inulin, xanthan gum and mixtures of two or more of these substances.

Among the above-mentioned coating materials, gelatine (in particular porcine, bovine, poultry and/or fish gelatine) is preferred, preferably having a swelling factor of greater than or equal to 20, preferably greater than or equal to 24. Gelatine is also particularly preferred as it is readily available and can be obtained with different swelling factors.

Also preferred are capsules made from maltodextrins (in particular based on cereals, especially maize, wheat, tapioca or potatoes), which preferably have DE values in the range from 10 to 20. Celluloses (e.g. cellulose ether), alginates (e.g. sodium alginate), carrageenan (e.g. beta-, iota-, lambda- and/or kappa-carrageenan), gum arabic, curdlan and/or agar agar or alginate capsules are also preferred.

In a further preferred embodiment, the shell of the capsules consists of melamine-formaldehyde resins or coacervation products of cationic monomers or biopolymers (such as chitosan) and anionic monomers, such as (meth)acrylates or alginates.

The capsules are generally finely dispersed liquid or solid phases coated with film-forming polymers, during the production of which the polymers are deposited on the material to be coated after emulsification and coacervation or interfacial polymerization. According to another method, melted waxes are incorporated in a matrix (“microsponge”), which can also be coated as microparticles with film-forming polymers. According to a third method, particles are alternately coated with polyelectrolytes of different charges (“layer-by-layer” method). The microscopically small capsules can be dried like powder.

In addition to mononuclear microcapsules, multinuclear aggregates, also known as microspheres, are also known, which contain two or more nuclei distributed in the continuous shell material. Mononuclear- or multinuclear microcapsules can also be enclosed by an additional second, third etc. shell. The shell can consist of natural, semi-synthetic or synthetic materials. Natural shell materials include gum arabic, agar-agar, agarose, maltodextrins, alginic acid or its salts, e.g., sodium or calcium alginate, fats and fatty acids, cetyl alcohol, collagen, chitosan, lecithin, gelatine, albumin, shellac, polysaccharides such as starch or dextran, polypeptides, protein hydrolysates, sucrose and waxes. Semi-synthetic coating materials include chemically modified celluloses, in particular cellulose esters and- ethers, e.g., cellulose acetate, ethyl cellulose, hydroxypropyl cellulose, hydroxypropyl methyl cellulose and carboxymethyl cellulose, as well as starch derivatives, in particular starch ethers and- esters. Synthetic coating materials are, for example, polymers such as polyacrylates, polyamides, polyvinyl alcohol or polyvinylpyrrolidone.

Examples of coating materials/shell materials of the prior art for the production of microcapsules are the following commercial products (the shell material is indicated in brackets in each case): Hallcrest Microcapsules (gelatine, gum arabic), Coletica Thalaspheres (marine collagen), Lipotec Millicapsules (alginic acid, agar-agar), Induchem Unispheres (lactose, microcrystalline cellulose, hydroxypropylmethylcellulose); Unicerin C30 (lactose, microcrystalline cellulose, hydroxypropylmethylcellulose), Kobo Glycospheres (modified starch, fatty acid esters, phospholipids), Softspheres (modified agar-agar) and Kuhs Probiol Nanospheres (phospholipids) as well as Primaspheres and Primasponges (chitosan, alginates) and Primasys (phospholipids).

The encapsulation of the active ingredients or flavoring mixtures according to the invention has the advantage that it protects the sensitive active ingredients and possibly other aroma and flavoring substances from environmental influences (light, oxygen, moisture) and ensures, for example, that oxidation is slowed down. In addition, encapsulation enables easy storage and preservation. Encapsulation also offers a longer shelf life for active ingredients or mixtures of active ingredients and prevents undesirable interactions between food ingredients and the corresponding active ingredients. Encapsulation enables a targeted and controlled release of the active ingredients at the right time. This guarantees the optimum taste experience at the time of consumption.

Additives and excipients, in particular preservatives, colorants, antioxidants, flow agents, thickeners, etc. can also be added to the active ingredients or flavoring preparations according to the invention.

Due to the advantageous properties described above, the present invention relates in a further aspect to the use of an active ingredient according to the invention or a flavoring preparation according to the invention as a flavoring agent. Here, the total amount of the active ingredient according to the invention or the mixture of active ingredients according to the invention or the flavoring or flavoring preparation is such as to produce an oral trigeminal stimulus, preferably a mouth-watering one in which the flow of saliva in the mouth is stimulated, or to trigger a tingling/fizzing effect in the mouth, when consumed or applied.

Due to the advantageous properties described above, the present invention also relates to the use of an active ingredient or flavoring preparation according to the invention, wherein the total amount of the active ingredient or flavoring preparation according to the invention is sufficient, to enhance or complement, during consumption or use thereof, pleasant taste properties of another active ingredient or aroma or flavoring agent, in particular that of an active ingredient or aroma or flavoring agent contained in the mixture or preparation, the taste impression being selected from the group consisting of warming, pungent or cooling notes.

Due to its advantageous properties described above, the present invention also relates to the use of an active ingredient or flavoring preparation according to the invention, wherein the total amount of the active ingredient(s) according to the invention is sufficient, to reduce, neutralize or mask unpleasant taste characteristics of another active ingredient or flavoring agent, in particular that of a substance contained in the mixture or preparation, during consumption or use thereof, the taste impression being selected from the group consisting of astringent, bitter, dry, dusty, floury, chalky or metallic notes. This makes it possible to reduce or eliminate known taste disadvantages of flavors, such as astringent or bitter notes, especially of sweeteners such as steviosides. In particular, the pungent, bitter or metallic aftertaste is reduced or masked even when small amounts of active ingredient or active ingredient mixture are added.

The active ingredients according to the invention or the flavoring preparations according to the invention with the trigeminal effects described above, in particular mouth-watering, tingling/fizzing effects, have a broad field of application, in particular in foods, in food supplements, cosmetic preparations, in particular in the oral care sector, or oral pharmaceutical preparations or animal feeds.

In particular, the active ingredients according to the invention or the flavoring preparations according to the invention are used for the manufacture of foodstuffs, food supplements, cosmetic preparations, preferably in the oral care sector, or pharmaceutical preparations or animal feeds, because of their advantageous properties and/or flavor-improving properties described above.

A further object of the present invention is therefore the use of one or more active ingredients according to the invention or a flavoring preparation according to the invention for the manufacture of foodstuffs, food supplements, cosmetic or pharmaceutical preparations, tobacco products or animal feed.

In a further aspect, the present invention therefore also comprises foodstuffs, food supplements, cosmetic or pharmaceutical preparations, tobacco products or animal feed comprising one or more active ingredient(s) or flavoring preparation(s) according to the invention.

The content of the active ingredient or flavoring preparation depends on the nature and use of the aforementioned products, wherein the active ingredient or mixture of active ingredients according to the invention or the flavor or flavoring preparation according to the invention is present in such a sufficient concentration to produce a trigeminal effect or an enhancing or modifying effect as described above.

Preferably, the final formulation or the finished product for use or consumption contains the active ingredient or the flavoring preparation typically in an amount of 0.001 ppm to 1000 ppm, based on the total weight of the final formulation.

A broad concentration range typically used to provide the desired level of sensitivity modulation is preferably 0.001 ppm to 1000 ppm, more preferably 0.01 ppm to 500 ppm, further preferably 0.1 ppm to 200 ppm, most preferably 1 ppm to about 150 ppm, most preferably 5 ppm to 100 ppm or 25 ppm to 50 ppm.

Preferably, the product according to the invention is
(i) a preparation for food or consumption selected from bakery products, confectionery, alcoholic or non-alcoholic beverages, instant beverages, meat products, eggs or egg products, cereal products, dairy products, fruit preparations, vegetable preparations, snacks, fat and oil-based products or emulsions thereof, other prepared dishes and soups, spices, seasonings, condiments, semi-finished products, food supplements; or
(ii) a preparation for oral hygiene, preferably based on a dental care product, and selected from the group consisting of: toothpaste, toothpaste, tooth gel, tooth powder, tooth brushing liquid, tooth brushing foam, mouthwash, toothpaste and mouthwash as a 2-in-1 product, lozenge, mouth spray, dental floss and dental care chewing gum; or
(iii) a pharmaceutical preparation, the preparation preferably being an oral pharmaceutical preparation, preferably in the form of capsules, tablets, coated tablets, granules, pellets, solid mixtures, dispersions in liquid phases, as emulsions, as powders, as solutions, as pastes or as other swallowable or chewable preparations.

Preferably, the foodstuffs are bakery products, for example bread, dry cookies, cakes, other baked goods, confectionery (for example chocolates, chocolate bar products, other bar products, fruit gums, hard and soft caramels, chewing gum), alcoholic or non-alcoholic drinks (e.g. coffee, tea, iced tea, wine, wine-based drinks, beer, beer-based drinks, liqueurs, schnapps, brandies, (carbonated) fruit-based soft drinks, (carbonated) isotonic drinks, (carbonated) soft drinks, nectars, spritzers, fruit and vegetable juices, fruit or vegetable juice preparations, instant drinks (e.g. instant cocoa drinks, instant tea drinks, instant coffee drinks, instant fruit drinks), meat products (e.g. ham, fresh sausage or raw sausage preparations, seasoned or marinated fresh or cured meat products), eggs or egg products (dried eggs, egg whites, egg yolks), cereal products (e.g. breakfast cereals, muesli bars, pre-cooked ready-to-eat rice products), dairy products (e.g. milk drinks, buttermilk drinks, milk ice cream, yoghurt, kefir, cream cheese, soft cheese, hard cheese, dried milk powder, whey, whey drinks, butter, buttermilk, partially or fully hydrolyzed milk protein-containing products), products made from soy protein or other soybean fractions (e.g. soy milk and products made from it, fruit drinks with soy protein, preparations containing soy lecithin, fermented products such as tofu or tempeh or products made from them), products made from other vegetable protein sources, for example oat protein drinks, fruit preparations (for example jams, fruit ice cream, fruit sauces, fruit fillings), products made from soy protein or other soybean fractions (for example soy milk and products made from it), fruit preparations (e.g. jams, fruit ice cream, fruit sauces, fruit fillings), vegetable preparations (e.g. ketchup, sauces, dried vegetables, frozen vegetables, pre-cooked vegetables, boiled vegetables), snacks (e.g. baked or deep-fried potato, potato chips or potato dough products, corn or peanut-based extrudates), fat- and oil-based products or emulsions thereof (e.g. mayonnaise, remoulade, dressings), other ready meals and soups (e.g. dry soups, instant soups, pre-cooked soups), spices, seasoning mixtures and, in particular, seasonings, which are used, for example, in snack foods: Seasonings), which are used in the snack sector, for example.

In addition to conventional food ingredients, the above-mentioned foodstuffs contain at least one effective amount of at least one active ingredient according to the invention or of a mixture of active ingredients according to the invention or of a flavoring preparation according to the invention.

Suitable excipients can be used to produce the preparations according to the invention, such as foodstuffs. Suitable excipients include, but are not limited to, emulsifiers, thickeners, food acids, acidity regulators, vitamins, antioxidants, flavor enhancers, food colorants and the like.

Emulsifiers: Emulsifiers are characterized by the important property of being soluble in both water and fat. Emulsifiers usually consist of a fat-soluble and a water-soluble part. They are always used when water and oil are to be mixed into a consistent, homogeneous mixture. Suitable emulsifiers used in the food processing industry are selected from: Ascorbyl palmitate (E 304), lecithin (E 322), phosphoric acid (E 338), sodium phosphate (E 339), potassium phosphate (E 340), calcium phosphate (E 341), magnesium orthophosphate (E 343), propylene glycol alginate (E 405), polyoxyethylene (8) stearate (E 430), polyoxyethylene stearate (E 431), ammonium phosphatides (E 442), sodium phosphate and potassium phosphate (E 450), sodium salts of fatty acids (E 470 a), mono- and diglycerides of fatty acids (E 471), acetic acid monoglycerides (E 472 a), lactic acid monoglycerides (E 472 b), citric acid monoglycerides (E 472 c), tartaric acid monoglycerides (E 472 d), diacetyl tartaric acid monoglycerides (E 472 e), sugar esters of fatty acids (E 473), sugar glycerides (E 474), polyglycerides of fatty acids (E 475), polyglycerol polyricinoleate (E 476), propylene glycol esters of fatty acids (E 477), sodium stearoyl lactylate (E 481), calcium stearoyl-2-lactylate (E 482), stearyl tartrate (E 483), sorbitan monostearate (E 491), stearic acid (E 570).

Thickening agents: Thickening agents are substances that are primarily able to bind water. The removal of unbound water leads to an increase in viscosity. From a characteristic concentration for each thickening agent, network effects also occur in addition to this effect, which usually lead to a disproportionate increase in viscosity. In this case, molecules are said to ‘communicate’ with each other, i.e. they become entangled. Most thickening agents are linear or branched macromolecules (e.g. polysaccharides or proteins) that can interact with each other through intermolecular interactions such as hydrogen bonds, hydrophobic interactions or ionic relationships. Extreme cases of thickening agents are layered silicates (bentonites, hectorites) or hydrated SiO₂ particles, which are present as dispersed particles and can bind water in their solid-like structure or interact with each other due to the interactions described. Examples are:
E 400—Alginic acid
E 401—sodium alginate
E 402—Potassium alginate
E 403—Ammonium alginate
E 404—Calcium alginate
E 405—Propylene glycol alginate
E 406—agar agar
E 407—Carrgeen, Furcelleran
E 407—carob bean gum
E 412—Guar gum
E 413—Traganth
E 414—Gum arabic
E 415—Xanthan gum
E 416—Karaya (Indian tragacanth)
E 417—Tara gum (Peruvian locust bean gum)
E 418—Gellan
E 440—Pectin, Opekta
E 440ii—Amidated pectin
E 460—microcrystalline cellulose, cellulose powder
E 461—Methylcellulose
E 462—Ethyl cellulose
E 463—Hydroxypropyl cellulose
E 465—Methyl ethyl cellulose
E 466—Carboxymethylcellulose, sodium carboxymethylcellulose

Food acids: The preparations according to the invention, such as foodstuffs, may contain carboxylic acids. Acids within the meaning of the invention are preferably acids permitted in foodstuffs, in particular those mentioned herein:
E 260—acetic acid
E 270—lactic acid
E 290—carbon dioxide
E 296—malic acid
E 297—fumaric acid
E 330—Citric acid
E 331—sodium citrate
E 332—potassium citrate
E 333—calcium citrate
E 334—tartaric acid
E 335—sodium tartrate
E 336—potassium tartrate
E 337—sodium potassium tartrate
E 338—phosphoric acid
E 353—metatartaric acid
E 354—calcium tartrate
E 355—adipic acid
E 363—succinic acid
E 380—triammonium citrate
E 513—sulfuric acid
E 574—gluconic acid
E 575—Glucono-delta-lactone

Acidity regulators: Acidity regulators are food additives that keep the acidity or alkalinity and thus the desired pH value of a food constant. They are usually organic acids and their salts, carbonates, and more rarely inorganic acids and their salts. The addition of an acidity regulator sometimes increases the stability and firmness of the food, causes a desired precipitation and improves the effect of preservatives. In contrast to acidifiers, they are not used to change the taste of food. Their effect is based on the formation of a buffer system in the food, in which the pH value does not change or changes only slightly when acidic or basic substances are added. Examples are:
E 170—Calcium carbonate
E 260-263—Acetic acid and acetates
E 270—Lactic acid
E 296—Malic acid
E 297—Fumaric acid
E 325-327—Lactate (lactic acid)
E 330-333—Citric acid and citrates
E 334-337—Tartaric acid and tartrates
E 339-341—orthophosphates
E 350-352—Malates (malic acid)
E 450-452—Di-, tri- and polyphosphates
E 500-504—Carbonates (carbonic acid)
E 507—Hydrochloric acid and chlorides
E 513-517—Sulfuric acid and sulfates
E 524-528—Hydroxides
E 529-530—Oxides
E 355-357—Adipic acid and adipates
E 574-578—Gluconic acid and gluconates

Vitamins: In a further embodiment of the present invention, the food additives may contain vitamins as a further optional group of additives. Vitamins have a wide variety of biochemical modes of action. Some act similarly to hormones and regulate mineral metabolism (e.g. vitamin D), or act on cell and tissue growth and cell differentiation (e.g. some forms of vitamin A). Others are antioxidants (e.g. vitamin E and, under certain circumstances, vitamin C). The majority of vitamins (e.g. the B vitamins) are precursors for enzymatic co-factors that help enzymes to catalyze certain metabolic processes. In this context, vitamins can sometimes be tightly bound to enzymes, for example as part of the prosthetic group: an example of this is biotin, which is part of the enzyme responsible for the formation of fatty acids. On the other hand, vitamins can also be less strongly bound and then act as co-catalysts, for example as groups that can be easily split off and transport chemical groups or electrons between the molecules. Folic acid, for example, transports methyl, formyl and methylene groups into the cell. Although its support in enzyme-substrate reactions is well known, its other properties are also of great importance for the body.

In the context of the present invention, suitable vitamins are substances selected from the group consisting of:
Vitamin A (retinol, retinal, beta-carotene),
Vitamin B₁ (thiamine),
Vitamin B₂ (Rioflavin),
Vitamin B₃ (niacin, niacinamide),
Vitamin B₅ (panthothenic acid),
Vitamin B₆ (pyridoxine, pyridoxamine, paridoxal),
Vitamin B₇ (biotin),
Vitamin B₉ (folic acid, folinic acid),
Vitamin B₁₂ (cyanobalamin, hydroxycobalmin, methylcobalmin),
Vitamin C (ascorbic acid),
Vitamin D (cholecalciferol),
Vitamin E (tocopherols, tocotrienols) and
Vitamin K (phylloquinone, menaquinone).
   In addition to ascorbic acid, the preferred vitamins are the vitamins of the tocopherol group.

Antioxidants: Both natural and artificial antioxidants are used in the food industry. The main difference between natural and artificial antioxidants is that the former occur naturally in food and the latter are produced artificially. For example, natural antioxidants, if they are to be used as food additives, are obtained from vegetable oils. Vitamin E—also known as tocopherol—is often produced from soybean oil, for example. Synthetic antioxidants such as propyl gallate, octyl gallate and dodecyl gallate, on the other hand, are obtained by chemical synthesis. Gallates can trigger allergies in sensitive people. Other antioxidants that can be used in compositions of the present invention are: Sulfur dioxide (E 220), sulfites Sodium sulfite (E 221), sodium hydrogen sulfite (E 222), sodium disulfite (E 223), potassium disulfite (E 224), calcium sulfite (E 226), calcium hydrogen sulfite (E 227), potassium hydrogen sulphite (E 228), lactic acid (E 270), ascorbic acid (E 300), sodium L-ascorbate (E 301), calcium L-ascorbate (E 302), ascorbic acid ester (E 304), tocopherol (E 306), alpha-tocopherol (E 307), gamma-tocopherol (E 308), delta-tocopherol (E 309), propyl gallate (E 310), octyl gallate (E 311), dodecyl gallate (E 312), isoascorbic acid (E 315), sodium isoascorbate (E 316), tertiary butylhydroquinone (TBHQ, E 319), butylhydroxianisole (E 320), butylhydroxitoluene (E 321), lecithin (E 322), citric acid (E 330), salts of citric acid (E 331 & E 332) sodium citrate (E 331), potassium citrate (E 332), calcium disodium EDTA (E 385), diphosphates (E 450), disodium diphosphate (E 450a), trisodium diphosphate (E 450b), tetrasodium diphosphate (E 450c), dipotassium diphosphate (E 450d), trepotassium diphosphate (E 450e), dipotassium diphosphate (E 450f), calcium dihydrogen diphosphate (E 450g), triphosphates (E 451), pentasodium triphosphate (E 451a), pentakalium triphosphate (E 451 b), polyphosphate (E 452), sodium polyphosphate (E 452a), potassium polyphosphate (E 452b), sodium calcium polyphosphate (E 452c), calcium polyphosphate (E 452d), tin II chloride (E 512).

Flavor enhancer: The preparations according to the invention, such as foodstuffs, may furthermore contain additional flavoring substances for enhancing a salty, possibly slightly sour and/or umami taste impression. The products according to the invention are thus used in combination with at least one further substance suitable for enhancing a pleasant taste impression (salty, umami, optionally slightly sour). Preferred compounds are salty-tasting compounds and salt-enhancing compounds. Preferred compounds are disclosed in WO 2007/045566. Umami compounds as described in WO 2008/046895 and EP 1 989 944 are also preferred.

Flavor corrigents: Furthermore, the preparations according to the invention, such as foodstuffs, may also comprise flavoring agents for masking bitter and/or astringent taste impressions (flavor corrigents). The (further) flavor corrigents are selected, for example, from the following list: Nucleotides (e.g. adenosine 5′-monophosphate, cytidine 5′-monophosphate) or their pharmaceutically acceptable salts, lactisols, sodium salts (e.g. sodium chloride, sodium lactate, sodium citrate, sodium acetate, sodium gluconoate), further hydroxyflavanones (e.g. e.g. eriodictyol, homoeriodictyol or their sodium salts), in particular according to US 2002/0188019, hydroxybenzoic acid amides according to DE 10 2004 041 496 (e.g. 2,4-dihydroxybenzoic acid vanillylamide, 2,4-dihydroxybenzoic acid N-(4-hydroxy-3-methoxybenzyl)amide, 2,4,6-trihydroxybenzoic acid N-(4-hydroxy-3-methoxybenzyl)amide, 2-hydroxybenzoic acid-N-4-(hydroxy-3-methoxybenzyl)amide, 4-hydroxybenzoic acid-N-(4-hydroxy-3-methoxybenzyl)amide, 2,4-dihydroxybenzoic acid-N-(4-hydroxy-3-methoxybenzyl)amide monosodium salt, 2,4-dihydroxybenzoic acid-N-2-(4-hydroxy-3-methoxyphenyl)-ethylamide, 2,4-dihydroxybenzoic acid-N-(4-hydroxy-3-ethoxybenzyl)amide, 2,4-dihydroxybenzoic acid-N-(3,4-dihydroxybenzyl)amide and 2-hydroxy-5-methoxy-N-[2-(4-hydroxy-3-methoxyphenyl)ethyl]amide (aduncamide), 4-hydroxybenzoic acid vanillylamide), bittermasking hydroxydeoxybenzoins, e.g. e.g. according to WO 2006/106023 (e.g. 2-(4-hydroxy-3-methoxyphenyl)-1-(2,4,6-tri,hydroxyphenyl)ethanone, 1-(2,4-dihydroxyphenyl)-2-(4-hydroxy-3-methoxyphenyl)ethanone, 1-(2-hydroxy-4-methoxyphenyl)-2-(4-hydroxy-3-methoxy-phenyl)ethanone), amino acids (e.g. gamma-aminobutyric acid according to WO 2005/096841 for reducing or masking an unpleasant taste impression such as bitterness), malic acid glycosides according to WO 2006/003107, salty-tasting mixtures according to PCT/EP 2006/067120, diacetyl trimers according to WO 2006/058893, mixtures of whey proteins with lecithins and/or bitter-masking substances such as ginger diones according to WO 2007/003527.

Flavoring agents: Preferred flavoring agents are those which cause a sweet odor impression, wherein the further flavoring agent(s) which cause a sweet odor impression are preferably selected from the group consisting of: Vanillin, ethyl vanillin, ethyl vanillin isobutyrate (=3 ethoxy-4-isobutyryloxybenzaldehyde), furaneol (2,5-dimethyl-4-hydroxy-3(2H)-furanone) and derivatives (e.g. homofuraneol, 2-ethyl-4-hydroxy-5-methyl-3(2H)-furanone), homofuronol (2-ethyl-5-methyl-4-hydroxy-3(2H)furanone and 5-ethyl-2-methyl-4-hydroxy-3(2H)-furanone), maltol and derivatives (e.g. ethylmaltol), coumarin and derivatives, gamma-lactones (e.g. gammaundecalactone, gamma-nonalactone), delta-lactones (e.g. 4-methyldeltalactone, massoilactone, deltadecalactone, tuberolactone), methyl sorbate, divanillin, 4-hydroxy-2(or 5)-ethyl-5(or 2)methyl-3(2H)furanone, 2-hydroxy-3-methyl-2-cyclopentenone, 3-hydroxy-4,5-dimethyl-2(5H)-furanone, fruit esters and fruit lactones (e.g. B. Acetic acid n-butyl ester, acetic acid isoamyl ester, propionic acid ethyl ester, butyric acid ethyl ester, butyric acid n-butyl ester, butyric acid i-soamyl ester, 3-methyl-butyric acid ethyl ester, n-hexanoic acid ethyl ester, n-hexanoic acid allyl ester, n-hexanoic acid n-butyl ester, n-octanoic acid ethyl ester, ethyl 3-methyl-3-phenyl glycidate, ethyl 2-trans-4-cis-decadienoate), 4-(p-hydroxyphenyl)-2-butanone, 1,1-dimethoxy-2,2,5-trimethyl-4-hexane, 2,6-dimethyl-5-hepten-1-al, 4-hydroxycinnamic acid, 4-methoxy-3-hydroxycinnamic acid, 3-methoxy-4-hydroxycinnamic acid, 2-hydroxycinnamic acid, 2,4-dihydroxybenzoic acid, 3-hydroxybenzoic acid, 3,4-dihydroxybenzoic acid, vanillic acid, homovanillic acid, vanillomandelic acid and phenylacetaldehyde.

Active substances for masking unpleasant taste sensations: Furthermore, the preparations according to the invention, such as foodstuffs, may also comprise further substances which serve to mask bitter and/or astringent taste impressions. These further flavor corrigents are selected, for example, from the following list: from nucleotides (e.g. adenosine 5-monophosphate, cytidine 5-monophosphate) or physiologically acceptable salts thereof, lactisols, sodium salts (e.g. sodium chloride, sodium lactate, sodium citrate, sodium acetate, sodium gluconoate), hydroxyflavanones, preferably eriodictyol, sterubin (eriodictyol-7-methylether), homoeriodictyol, and their sodium, potassium, calcium, magnesium or zinc salts (e.g. sodium chloride, sodium lactate, sodium citrate, sodium acetate, sodium gluconoate), magnesium or zinc salts thereof (in particular those as described in EP 1258200 A2), hydroxybenzoic acid amides, preferably 2,4-dihydroxybenzoic acid vanillylamide, 2,4-dihydroxybenzoic acid N-(4-hydroxy-3-methoxybenzyl)amide, 2,4,6-trihydroxybenzoic acid-N-(4-hydroxy-3-methoxybenzyl)amide, 2-hydroxybenzoic acid-N-4-(hydroxy-3-methoxybenzyl)amide, 4-hydroxybenzoic acid-N-(4-hydroxy-3-methoxybenzyl)amide, 2,4-dihydroxybenzoic acid-N-(4-hydroxy-3-methoxybenzyl)amide mono-sodium salt, 2,4-dihydroxybenzoic acid-N-2-(4-hydroxy-3-methoxy-phenyl)ethylamide, 2,4-dihydroxybenzoic acid-N-(4-hydroxy-3-ethoxybenzyl)amide, 2,4-dihydroxybenzoic acid-N-(3,4-dihydroxybenzyl)amide and 2-hydroxy-5-methoxy-N-[2-(4-hydroxy-3-methoxyphenyl)ethyl]amide; 4-hydroxybenzoic acid vanillylamides (in particular those as described in WO 2006/024587); hydroxydeoxybenzoines, preferably 2-(4-hydroxy-3-methoxyphenyl)-1-(2,4,6-trihydroxyphenyl)ethanone, 1-(2,4-dihydroxyphenyl)-2-(4-hydroxy-3-methoxyphenyl)-ethanone and 1-(2-hydroxy-4-methoxyphenyl)-2-(4-hydroxy-3-methoxyphenyl)ethanone) (in particular those as described in WO 2006/106023); hydroxyphenylalkanediones, such as gingerdione-[2], gingerdione-[3], gingerdione-[4], dehydrogingerdione-[2], dehydrogingerdione-[3], dehydrogingerdione-[4]); diacetyltrimers; gamma-aminobutyric acids; divanillins and 4-hydroxydihydrochalcones, in particular phloretin and Davidigenin, amino acids or mixtures of whey proteins with lecithins, hesperetin, 4-hydroxydihydrochalcones, or propenylphenylglycosides (chavicolglycosides), or extracts from Rubus suavissimus, extracts from Hydrangea macrophylla, pellitorin and derived aroma compositions, umami compounds, vanillyllignans, enterodiol, as well as N-decadienoylamino acids and mixtures thereof.

Food colorants: Food colorants, or dyes for short, are food additives used to color food. Colorants are divided into the groups of natural colorants and synthetic colorants. Nature-identical colorants are also of synthetic origin. Nature-identical colorants are synthetic imitations of naturally occurring coloring substances. Suitable colorants for use in the present composition are selected from: Curcumin (E 100), riboflavin (lactoflavin, lactoflavin, vitamin B2, E 101), tartrazine (E 102), quinoline yellow (E 104), yellow orange S (yellow orange RGL, E 110), cochineal (carminic acid, carmine, E 120), azorubine (carmoisine, E 122), amaranth (E 123), cochineal red A (Ponceau 4 R, Victoria scarlet 4 R, E 124), erythrosine (E 127), allura red AC (E 129), Patent blue V (E 131), indigotine (indigo carmine, E 132), brilliant blue FCF (patent blue AE, amido blue AE, E 133), chlorophylls, chlorophyllins (E 140), copper complexes of chlorophylls, Copper-chlorophyllin complex (E 141), brilliant acid green (green S, E 142), sugar caramel (sugar couleur, E 150 a), sulphite lye sugar caramel (E 150 b), ammonia sugar caramel (E 150 c), Ammonium sulphite caramel (E 150 d), brilliant black FCF, brilliant black PN, black PN (E 151), vegetable charcoal (E 153), brown FK (E 154), brown HT (E 155), carotene (carotene, E 160 a), annatto (bixin, norbixin, E 160 b), capsanthin (capsorubin, E 160 c), lycopene (E 160 d), beta-apo-8′-carotene (apocarotene, beta-apocarotene, E 160 e), Beta-apo-8′-carotenic acid ethyl ester (C30), apocarotene ester, beta-carotenic acid ester (E 160 f), lutein (xanthophyll, E 161 b), canthaxanthin (E 161 g), betanin, betene red (E 162), Anthocyanins (E 163), calcium carbonate (E 170), titanium dioxide (E 171), iron oxides, iron hydroxides (E 172), aluminum (E 173), silver (E 174), gold (E 175), litholrubin BK, ruby pigment BK (E 180).

A further object of the invention relates to cosmetic or pharmaceutical preparations which contain either an active ingredient according to the invention or a mixture of active ingredients or a flavoring preparation according to the invention.

Due to their special properties described above, the active ingredients or mixtures of active ingredients or flavoring preparations according to the invention are advantageously and preferably used primarily in oral hygiene products.

Oral hygiene products according to the invention can be formulated in a manner known per se, e.g., as toothpaste, toothpaste, toothpaste gel, tooth powder, toothbrushing liquid, toothbrushing foam, aqueous or aqueous-alcoholic oral care products (mouthwash), mouthwash as a 2-in-1 product, lozenge, mouth spray, dental floss and dental care chewing gum.

Toothpastes or toothpastes which are flavored with the active ingredients or active ingredient mixtures or flavoring preparations according to the invention are generally understood to be gel-like or pasty preparations of water, thickening agents, humectants, abrasive or cleaning agents, surfactants, sweeteners, flavoring agents, deodorizing active ingredients and active ingredients against oral and dental diseases. All conventional cleaning agents, such as chalk, dicalcium phosphate, insoluble sodium metaphosphate, aluminum silicates, calcium pyrophosphate, finely divided synthetic resins, silicas, aluminum oxide and aluminum oxide trihydrate can be used in the toothpastes according to the invention.

Preferably suitable cleaning agents for the toothpastes according to the invention are above all finely divided xerogel silicas, hydrogel silicas, precipitated silicas, aluminum oxide trihydrate and finely divided alpha-aluminum oxide or mixtures of these cleaning agents in amounts of 15 to 40% by weight of the toothpaste. Low-molecular polyethylene glycols, glycerol, sorbitol or mixtures of these products in quantities of up to 50% by weight can be used as humectants. Among the known thickening agents are the thickening, finely divided gel silicas and hydrocolloids, e.g., carboxymethylcellulose, hydroxyethylcellulose, hydroxypropylguar, hydroxyethyl starch, polyvinylpyrrolidone, high molecular weight polyethylene glycol, plant gums such as tragacanth, agar-agar, carragheen moss, gum arabic, xantham gum and carboxyvinyl polymers (e.g. Carbopol® types) are suitable. In addition to the mixtures of menthofuran and menthol compounds, the oral and dental care compositions may in particular contain surface-active substances, preferably anionic and non-ionic high-foaming surfactants, such as the substances already mentioned above, but in particular alkyl ether sulphate salts, alkyl polyglucosides and mixtures thereof.

Other common toothpaste additives are:
Preservatives and antimicrobial substances such as p-hydroxybenzoic acid methyl, ethyl or propyl ester, sodium sorbate, sodium benzoate, bromochlorophene, phenylsalicylic acid ester, thymol and the like,
Anti-tartaric agents, e.g., organophosphates such as 1-hydroxyethane-1,1-diphosphonic acid, 1-phosphonopropane-1,2,3-tricarboxylic acid and others, which are known, for example, from U.S. Pat. No. 3,488,419, DE 2224430 A1 and DE 2343196 A1;
other caries-inhibiting substances such as sodium fluoride, sodium monofluorophosphate, stannous fluoride;
Sweeteners, such as saccharin sodium, sodium cyclamate, sucrose, lactose, maltose, fructose or Aspartame®, (L-aspartyl-L-phenylalanine methyl ester), stevia extracts or their sweetening ingredients, in particular rebaudiosides;
additional flavors such as eucalyptus oil, anise oil, fennel oil, caraway oil, methyl acetate, cinnamaldehyde, anethole, vanillin, thymol and mixtures of these and other natural and synthetic flavors;
Pigments such as titanium dioxide;
Colorants;
Buffer substances such as primary, secondary or tertiary alkali phosphates or citric acid/sodium citrate;
wound-healing and anti-inflammatory substances such as allantoin, urea, azulene, chamomile active ingredients and acetylsalicylic acid derivatives.

Hydrotropes such as ethanol, isopropyl alcohol or polyols can also be used to improve the flow behavior; these substances largely correspond to the carriers described at the beginning. Polyols that come into consideration here preferably have 2 to 15 carbon atoms and at least two hydroxyl groups. The polyols may also contain other functional groups, in particular amino groups, or be modified with nitrogen. Typical examples are:
Glycerine;
Alkylene glycols, such as ethylene glycol, diethylene glycol, propylene glycol, butylene glycol, hexylene glycol and polyethylene glycols with an average molecular weight of 100 to 1000 Daltons;
technical oligoglycerol mixtures with an inherent degree of condensation of 1.5 to 10, such as technical diglycerol mixtures with a diglycerol content of 40 to 50% by weight;
Methyol compounds, in particular trimethylolethane, trimethyloylpropane, trimethylolbutane, pentaerythritol and dipentaerythritol;
Lower alkyl glucosides, in particular those with 1 to 8 carbons in the alkyl group, such as methyl and butyl glucoside;
Sugar alcohols with 5 to 12 carbon atoms, such as sorbitol or mannitol,
Sugar with 5 to 12 carbon atoms, such as glucose or sucrose;
Amino sugars, such as glucamine;
Dialcohol amines, such as diethanolamine or 2-amino-1,3-propanediol.

Suitable preservatives include phenoxyethanol, formaldehyde solution, parabens, pentanediol or sorbic acid as well as the silver complexes known as Surfacine® and other classes of substances known and suitable to the skilled person.

Perfume oils are those already defined above. In particular, peppermint oil, spearmint oil, anise oil, star anise oil, caraway oil, eucalyptus oil, fennel oil, lemon oil, wintergreen oil, clove oil, menthol and the like may be considered as aromas.

A preferred embodiment of the cosmetic preparations are toothpastes in the form of an aqueous, pasty dispersion containing polishing agents, humectants, viscosity regulators and optionally other customary components, as well as the mixture of menthofuran and menthol compounds in amounts of 0.5 to 2% by weight.

In mouthwashes, a combination with aqueous-alcoholic solutions of different grades of essential oils, emulsifiers, astringent and tonifying drug extracts, tartar-inhibiting, antibacterial additives and taste corrigents is readily possible. A further preferred embodiment of the invention is a mouthwash in the form of an aqueous or aqueous-alcoholic solution containing the mixture of menthofuran and menthol compounds in amounts of 0.05 to 0.4% by weight. In mouthwashes which are diluted before use, sufficient effects can be achieved with higher concentrations according to the intended dilution ratio.

Oral care preparations according to the invention contain, based on the total weight of the composition, preferably 0.1 ppm to 1000 ppm, preferably 1 ppm to 500 ppm, of at least one active ingredient or a mixture of active ingredients or a flavoring preparation according to the present invention.

The total content of the active ingredient or of the several active ingredients according to the invention or of the flavoring preparation according to the present invention in ready-to-use mouthwashes is preferably 0.01 to 1% by weight, preferably 0.05 to 0.5% by weight, particularly preferred is a content of 0.1 to 0.3% by weight, in each case based on the total mouthwash.

In mouthwash concentrates, the total content of the one or more active ingredients according to the invention is 0.1 to 15% by weight, preferably a content of 0.5 to 8% by weight, particularly preferably 1 to 5% by weight, in each case based on the total mouthwash concentrate.

In toothpastes, the total content of the one or more active ingredients according to the invention is 0.1 to 5% by weight, preferably 0.5 to 2% by weight, particularly preferably 0.8 to 1.5% by weight, in each case based on the total toothpaste.

A further positive property of the compounds or active ingredients according to the invention is their stability in toothpastes based on chalk or bicarbonate, which are difficult to aromatize due to their alkaline pH value.

The present invention also includes chewing gum comprising one or more cooling agents according to the invention or a cooling agent mixture according to the invention or a flavoring preparation according to the invention.

Chewing gums, as a further example of oral care compositions flavored with the compositions containing the active ingredients or active ingredients according to the invention, generally comprise a chewing gum base, i.e. a chewing mass which becomes plastic when chewed, sugars of various types, sugar substitutes, other sweet-tasting substances, sugar alcohols (in particular sorbitol, xylitol, mannitol), cooling agents, flavoring agents for unpleasant taste sensations, other flavor modulators for further, generally not unpleasant taste sensations, flavor-modulating substances e.g., inositol phosphate, nucleotides such as guanosine monophosphate, adenosine monophosphate or other substances such as sodium glutamate or 2-phenoxypropionic acid), humectants, thickeners, emulsifiers and stabilizers.

Chewing gum compositions typically contain a water-insoluble and a water-soluble component. The water-insoluble base, also known as the “gum base”, usually comprises natural or synthetic elastomers, resins, fats and oils, plasticizers, fillers, colorants and optionally waxes. The proportion of the base in the total composition is usually 5 to 95% by weight, preferably 10 to 50% by weight and in particular 20 to 35% by weight. In a typical embodiment of the invention, the base is composed of 20 to 60% by weight of synthetic elastomers, 0 to 30% by weight of natural elastomers, 5 to 55% by weight of plasticizers, 4 to 35% by weight of fillers and, in minor amounts, additives such as colorants, antioxidants and the like, with the proviso that they are water-soluble in small amounts at most.

In addition to traditionally used natural resins or the natural latex chicle, common chewing gum bases today mostly comprise elastomers such as polyvinyl acetates (PVA), polyethylenes, (low or medium molecular weight) polyisobutenes (PIB), polybutadienes, isobutene-isoprene copolymers (butyl rubber), polyvinyl ethyl ether (PVE), polyvinyl butyl ether, copolymers of vinyl esters and vinyl ethers, styrene-butadiene copolymers (styrene-butadiene rubber, SBR) or vinyl elastomers, e.g. based on vinyl acetate/vinyl laurate, vinyl acetate/vinyl stearate or ethylene/vinyl acetate, as well as mixtures of the aforementioned elastomers. In addition, chewing gum bases include other ingredients such as (mineral) fillers, plasticizers, emulsifiers, antioxidants, waxes, fats or fatty oils, such as hardened (hydrogenated) vegetable or animal fats, mono-, di- or triglycerides. Suitable (mineral) fillers include calcium carbonate, titanium dioxide, silicon dioxide, talc, aluminum oxide, dicalcium phosphate, tricalcium phosphate, magnesium hydroxide and mixtures thereof. Suitable plasticizers or detackifiers are, for example, lanolin, stearic acid, sodium stearate, ethyl acetate, diacetin (gylcerol diacetate), triacetin (gylcerol triacetate), triethyl citrate. Suitable waxes are, for example, kerosene waxes, candelilla wax, carnauba wax, microcrystalline waxes and polyethylene waxes. Suitable emulsifiers include phosphatides such as lecithin, mono- and diglycerides of fatty acids, e.g., glycerol monostearate. Suitable sweeteners include both sugar-containing and sugar-free compounds, which are used in amounts of 5 to 95, preferably 20 to 80 and in particular 30 to 60% by weight based on the chewing gum composition. Typical saccharide sweeteners are sucrose, dextrose, maltose, dextrin, dried invert sugar, fructose, levulose, galactose, corn syrup and mixtures thereof. Sorbitol, mannitol, xylitol, hardened starch hydrolysates, maltitol and mixtures thereof can be used as sugar substitutes. Furthermore, so-called HIAS (“High Intensity Artificial Sweeteners”), such as sucralose, aspartame, acesulfame salts, alitame, saccharin and saccharin salts, cyclamic acid and its salts, glycyrrhizine, dihydrochalcones, thaumatin, monellin and the like alone or in mixtures, can also be considered as additives.

Suitable synthetic elastomers include polyisobutylenes with average molecular weights (according to GPC) of 10,000 to 100,000 and preferably 50,000 to 80,000, isobutylene-isoprene copolymers (butyl elastomers), styrene-butadiene copolymers (styrene:butadiene ratio e.g. 1:3 to 3:1), polyvinyl acetates with average molecular weights (according to GPC) of 2,000 to 90,000 and preferably 10,000 to 65,000, polyisoprenes, polyethylene, vinyl acetate-vinyl laurate copolymers and mixtures thereof.

Examples of suitable natural elastomers are rubbers such as smoked or liquid latex or guayule as well as natural rubbers such as jelutong, lechi caspi, perillo, sorva, massaranduba balata, massaranduba chocolate, nispero, rosindinba, chicle, gutta hang 1kang and mixtures thereof. The choice of synthetic and natural elastomers and their mixing ratios depends essentially on whether the chewing gums are intended to produce bubbles (“bubble gums”) or not. Elastomer mixtures containing Jelutong, Chicle, Sorva and Massaranduba are preferably used.

In most cases, the elastomers prove to be too hard or not malleable enough during processing, so that it has proven to be advantageous to use special plasticizers, which of course must also meet all the requirements for approval as food additives. In this respect, esters of resin acids are particularly suitable, for example esters of lower aliphatic alcohols or polyols with fully or partially hardened, monomeric or oligomeric resin acids. In particular, methyl, glycerol or pentareythritol esters and mixtures thereof are used for this purpose. Alternatively, terpene resins, which can be derived from alpha-pinene, beta-pinene, delta-limonene or mixtures thereof, can also be considered.

Possible fillers or texturizing agents include magnesium or calcium carbonate, ground pumice, silicates, especially magnesium or aluminum silicates, clays, aluminum oxides, talc, titanium dioxide, mono-, di- and tricalcium phosphate and cellulose polymers.

Suitable emulsifiers are tallow, hardened tallow, hardened or partially hardened vegetable oils, cocoa butter, partial glycerides, lecithin, triacetin and saturated or unsaturated fatty acids with 6 to 22 and preferably 12 to 18 carbon atoms and mixtures thereof.

Possible colorants and whitening agents include the FD and C types approved for food coloring, plant and fruit extracts and titanium dioxide.

The base compounds can contain waxes or be wax-free.

In addition to the water-insoluble gum base, chewing gum preparations regularly contain a water-soluble portion, which is formed, for example, by softeners, sweeteners, fillers, flavorings, flavor enhancers, emulsifiers, colorants, acidifiers, antioxidants and the like, with the proviso that the ingredients have at least sufficient water solubility. Depending on the water solubility of the specific representatives, individual components can therefore belong to both the water-insoluble and the water-soluble phase. However, it is also possible to use combinations of, for example, a water-soluble and a water-insoluble emulsifier, whereby the individual agents are then in different phases. Typically, the water-insoluble portion makes up 5 to 95% by weight and preferably 20 to 80% by weight of the preparation.

Water-soluble softeners or plasticizers are added to chewing gum compositions to improve chewability and chewing sensation and are typically present in the mixtures in amounts of 0.5 to 15% by weight. Typical examples are glycerol, lecithin and aqueous solutions of sorbitol, hardened starch hydrolysates or corn syrup.

Both sugar-containing and sugar-free compounds are suitable as sweeteners, which are used in amounts of 5 to 95% by weight, preferably 20 to 80% by weight and in particular 30 to 60% by weight based on the chewing gum composition. Typical saccharide sweeteners are sucrose, dextrose, maltose, dextrin, dried invert sugar, fructose, levulose, galactose, corn syrup and mixtures thereof. Sorbitol, mannitol, xylitol, hardened starch hydrolysates, maltitol and mixtures thereof can be used as sugar substitutes. Furthermore, so-called HIAS (“High Intensity Artificial Sweeteners”), such as sucralose, aspartame, acesulfame salts, alitame, saccharin and saccharin salts, cyclamic acid and its salts, glycyrrhizine, dihydrochalcones, thaumatin, monellin and the like alone or in mixtures, can also be considered as additives. Particularly effective are also the hydrophobic HIAS, which are the subject of the international patent application WO 2002 091849 A1 (Wrigleys), as well as stevia extracts and their active ingredients, in particular ribeaudioside A. The amount of these substances used depends primarily on their performance and is typically in the range of 0.02 to 8% by weight.

Fillers such as polydextrose, raftilose, rafitilin, fructooligosaccharides (NutraFlora), palatinose oligosaaccharides, guar gum hydrolysates (Sun Fiber) and dextrins are particularly suitable for the production of low-calorie chewing gum.

The choice of further flavorings is practically unlimited and is not critical to the essence of the invention. Typically, the total amount of all flavorings is 0.1 to 15% by weight and preferably 0.2 to 5% by weight, based on the chewing gum composition. Suitable further flavoring agents are, for example, essential oils, synthetic flavors and the like, such as anise oil, star anise oil, caraway oil, eucalyptus oil, fennel oil, lemon oil, wintergreen oil, clove oil, and the like, as are also used, for example, in oral and dental care compositions.

In chewing gums, the total content of the active ingredient or of the several active ingredients or of the flavoring preparation according to the present invention is 0.01 to 2.5% by weight, preferably 0.1 to 2.0% by weight, particularly preferably 0.5 to 1.5% by weight, even more preferably 0.8 to 1.0% by weight, in each case based on the total chewing gum.

In lozenges (hard candies), the total content of the active ingredient or of the plurality of active ingredients or of the flavoring preparation according to the present invention is 0.01 to 2.5% by weight, preferably 0.05 to 2.5% by weight, particularly preferably 0.07 to 1.0% by weight, even more preferably 0.08 to 0.3% by weight, in each case based on the entire lozenge.

The use of the active ingredients according to the invention or the mixture of active ingredients according to the invention or the flavouring preparation according to the invention in oral care products (oral hygiene products), such as mouthwashes and toothpastes, and chewing gums, results in the salivary flow being stimulated and unpleasant, especially bitter or metallic, taste impressions being partially masked or neutralized, which are caused, for example by substances such as triclosan, zinc citrate, -sulphate, poly- and pyrophosphates, bicarbonates, strontium and potassium salts, tin pyrophosphate, -chloride, aluminum lactate, hydrogen peroxide, fluorides, vitamins, cetylpyridinium chloride and emulsifiers, e.g. particularly sodium lauryl sulphate, sodium lauryl sarcosinate and cocamidopropyl betaine, and sweeteners such as aspartame, saccharin, acesulfame-K, sorbitol; xylitol, cyclamates (e.g. sodium cyclamate), sucralose, alitam, neotam, thaumatin, neohesperidin dihydrochalcone, maltitol, lactitol or chewing gum masses.

Due to the salivation stimulating effect, the teaching according to the invention also includes the use of the active ingredients described herein for medical purposes, preferably in pharmaceutical compositions for the treatment of an individual, preferably a mammal, in particular a human, farm animal or pet, of dry mouth or xerostomia.

A further object of the invention therefore relates to pharmaceutical preparations which contain either an active ingredient according to the invention or a mixture of active ingredients according to the invention or a flavoring preparation according to the invention.

In a further aspect, the present invention therefore relates to a pharmaceutical preparation comprising either an active ingredient according to the invention or a mixture of active ingredients according to the invention or a flavoring preparation according to the invention, for use as a medicament, preferably for use in the prevention and/or treatment of dry mouth or xerostomia.

Dry mouth or xerostomia can have various causes: Dry mouth is caused by too little saliva flow (hyposalivation) which is a common side effect of medications such as antichloinergics, diuretics or psychotropic drugs. It can also be caused by diseases such as sialladenitis, Sjögren's syndrome, sicca syndrome or Heerfordt's syndrome. In addition, the removal of salivary glands, radiotherapy, febrile general illnesses, the use of tetrahydrocannabinol or chronic stress with increased sympathetic tone can lead to dry mouth. Another cause of dry mouth can be increased mouth breathing. The highly viscous saliva produced by dry mouth can cause inflammation in the ducts of the salivary glands; the sublingual salivary gland is particularly affected. The direct (unpleasant) consequences are dryness of the oral mucosa and difficulty swallowing.

This results in problems when speaking and eating dry food. It can also be difficult to fall asleep. Chronic sufferers usually suffer in terms of their dental health. Caries is a frequent consequence in the absence of the protective accompanying substances of normal saliva flow. Normal oral saliva contains a number of antimicrobial components.

Dry mouth can be eliminated or at least reduced by oral administration of the active ingredients or active ingredient mixtures according to the invention. For this purpose, the active ingredients or mixtures of active ingredients are administered in the form of pharmaceutical compositions comprising a pharmaceutically acceptable excipient with at least one active ingredient according to the invention and optionally further active ingredients. These compositions are preferably administered orally.

Examples of suitable pharmaceutical formulations or compositions are solid dosage forms such as powders, powders, granules, tablets, pastilles, sachets, cachets, coated tablets, capsules such as hard and soft gelatine capsules, as well as liquid dosage forms such as solutions, emulsions, in particular oil-in-water emulsions, suspensions, for example lotions, etc., preferably preparations that can be sucked or chewed. Particularly preferred pharmaceutical preparations are non-prescription products and over-the-counter medicines, so-called OTC (“over the counter”) preparations.

In the preparation of the cosmetic or pharmaceutical compositions according to the invention, the active ingredient(s) or the mixture of active ingredients according to the invention are usually mixed or diluted with an excipient. Excipients can be solid, semi-solid or liquid materials that serve as a vehicle, carrier or medium for the active ingredient. The active ingredient content (of an active ingredient or several active ingredients according to the invention contained simultaneously) can vary over a wide range and is approximately, in each case based on the total weight of the preparation, from about 0.05 ppm to 10% by weight, preferably 0.1 ppm to 10% by weight.

Suitable excipients include, for example, lactose, dextrose, sucrose, sorbitol, mannitol, starches, acacia gum, calcium phosphate, alginates, tragacanth, gelatin, calcium silicate, microcrystalline cellulose, polyvinylpyrrolidone, cellulose, water, syrup and methylcellulose. Furthermore, the formulations may contain pharmaceutically acceptable carriers or conventional excipients, such as lubricants, for example tallow, magnesium stearate and mineral oil, wetting agents, emulsifying and suspending agents, preservatives, such as methyl and propyl hydroxybenzoates; antioxidants; anti-irritants; chelating agents; coating agents; emulsion stabilizers; film formers; gel formers; odor masking agents; taste corrigents; resins; hydrocolloids; solvents; solubilizers; neutralizing agents; permeation accelerators; pigments; quaternary ammonium compounds; refatting and superfatting agents; ointment, cream or oil bases; silicone derivatives; spreading aids; stabilizers; sterilants; suppository bases; tablet excipients, such as binders, fillers, lubricants, disintegrants or coatings; propellants; drying agents; opacifiers; thickeners; waxes; plasticizers; white oils. A relevant embodiment is based on expert knowledge and is adequately described in the relevant technical literature.

The preparations according to the invention may also contain cosmetically and/or dermatologically and/or pharmacologically active ingredients in addition to conventional additives or auxiliaries. Non-limiting examples of suitable further active ingredients are to be mentioned:

Cosmetically and/or dermatologically active ingredients: Suitable cosmetically and/or dermatologically active ingredients are e.g. coloring active ingredients, skin and hair pigmentation agents, tinting agents, tanning agents, bleaching agents, keratin-hardening substances, antimicrobial active ingredients, light filter active ingredients, repellent active ingredients, substances with a hyperemizing effect, keratolytic and keratoplastic active ingredients, anti-dandruff active ingredients, anti-inflammatory agents, keratinizing active ingredients, antioxidant or active ingredients that act as radical scavengers, substances that moisturize or moisturize the skin, lipid-replenishing active ingredients, active ingredients with anti-erythimatic or anti-allergic properties, branched fatty acids such as 18-methyleicosanoic acid, and mixtures thereof. Artificial skin-tanning active agents which are suitable for tanning the skin without natural or artificial irradiation with UV rays; these are e.g., dihydroxyacetone, alloxan and walnut shell extract. Suitable keratin-hardening substances are generally active ingredients such as those used in antiperspirants, e.g., potassium aluminum sulfate, aluminum hydroxychloride, aluminum lactate, etc.

Antimicrobial agents: Antimicrobial agents are used to destroy microorganisms or inhibit their growth. They therefore serve both as a preservative and as a deodorizing substance that reduces the development or intensity of body odour. These include, for example, conventional preservatives known to the skilled person, such as p-hydroxybenzoic acid esters, imidazolidinyl urea, formaldehyde, sorbic acid, benzoic acid, salicylic acid, etc. Such deodorizing substances are e.g. zinc ricinoleate, triclosan, undecylenic acid alkylolamides, citric acid triethyl ester, chlorhexidine etc.

Excipients and additives: Suitable excipients and additives for the manufacture of hair cosmetic or skin cosmetic preparations are familiar to the skilled person and can be taken from cosmetic manuals, i.e. the relevant specialist literature. The added excipients and additives are preferably cosmetically and/or pharmaceutically acceptable excipients. Pharmaceutically acceptable excipients are those known to be usable in the field of pharmacy, food technology and related areas, in particular those listed in relevant pharmacopoeias (e.g., DAB, Ph. Eur., BP, NF) as well as other excipients whose properties do not conflict with physiological use.

Suitable excipients can be: Lubricants, wetting agents, emulsifying and suspending agents, preservatives, antioxidants, anti-irritants, chelating agents, emulsion stabilizers, film formers, gelling agents, odor masking agents, hydrocolloids, solvents, solubilizers, neutralizing agents, permeation accelerators, pigments, quaternary ammonium compounds, refatting and superfatting agents, ointment, cream or oil base substances, silicone derivatives, stabilizers, sterilants, blowing agents, drying agents, opacifying agents, thickening agents, waxes, plasticizers, white oil. The relevant formulation is based on expert knowledge, as can be found in the relevant specialist literature.

Other suitable additives are selected from perfume oils, hair polymers, hair and skin conditioners, graft polymers, water-soluble or dispersible silicone-containing polymers, light stabilizers, bleaching agents, care products, colorants, tinting agents, tanning agents, dyes, consistency agents, humectants, refatting agents, collagen, protein hydrolysates, lipids, antioxidants, defoamers, antistatic agents, emollients, emollients, plasticizers, peroxide decomposers.

The present invention also relates to tobacco products.

The active ingredients according to the invention, i.e., the mouth-watering and/or tingling/fizzing active ingredient according to the invention, or the mixture of active ingredients according to the invention or the flavoring preparation according to the invention, can advantageously also be used for the manufacture of tobacco products. Examples of such tobacco products include cigars, cigarettes, pipe tobacco, chewing tobacco, and snuff. The manufacture of tobacco products supplemented with cooling additives is known per se.

Further aspects of the present invention are apparent from the following examples and the appended patent claims.

### EXAMPLES

The examples serve to illustrate the invention without limiting it. Unless otherwise stated, all data refer to the weight.

### Examples of Synthesis

The active compounds according to the invention were prepared on the basis of synthesis methods as described in more detail below and known to the person skilled in the art in the field of organic synthesis.

The active ingredients according to the invention are synthesized according to the following reaction schemes:

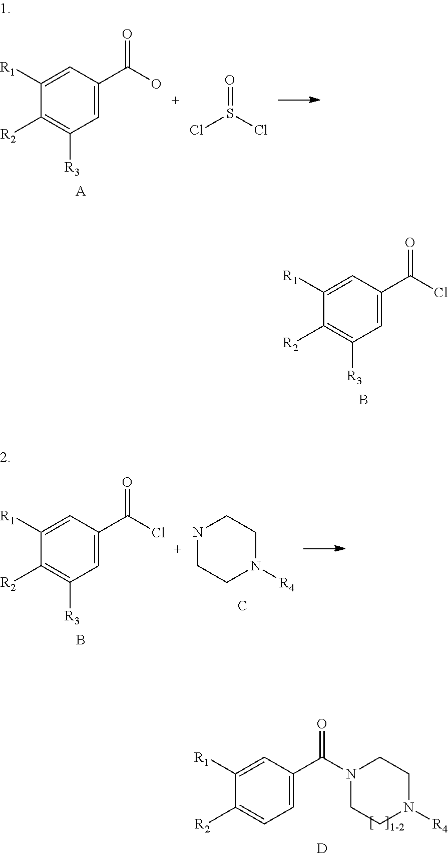

for the compounds in which
R₁ stands for H, ethoxy, methoxy, methyl ethoxy, propoxy, hydroxy;
R₂ stands for H, ethoxy, methoxy, methyl ethoxy, propoxy, hydroxy;
R₃ stands for H; methoxy;
R₁-R₂ stand for methylenedioxo; and
R₄ stands for:

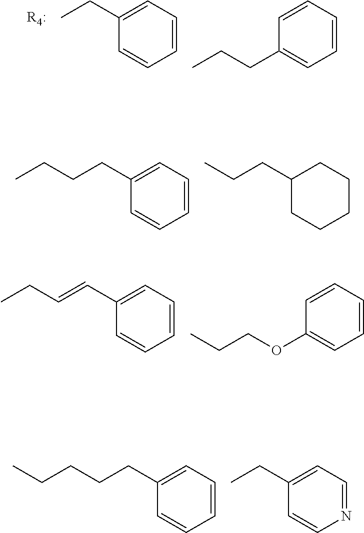

Method A: The benzoic acid derivative A was placed in toluene, thionyl chloride was added and stirred for 1 h under reflux. The mixture was allowed to cool to approx. 50° C. and the mixture was concentrated. The residue B is used in the 2nd stage. The piperazine derivative C was introduced with triethylamine and toluene. The residue B from the 1st stage was added and stirred for 4 to 16 h at room temperature.

The mixture was quenched with water and the phases separated. The organic phase was dried with Na₂SO₄, filtered and concentrated. The residue was chromatographed with cyclohexane: ethyl acetate (+0.2% triethylamine) over silica gel.

Method B: The benzoic acid derivative A was placed in dichloromethane. Oxalyl chloride was added dropwise. After gas evolution was complete, stirring was continued for 30 min at reflux and 30 min under reflux. After cooling, the mixture was concentrated, and the residue was used in the 2nd stage.

### Variant I:

The piperazine derivative C and triethylamine were added to dichloromethane. The residue B of the 1st stage was dissolved in dichloromethane and added dropwise. The mixture was stirred for 24 h at room temperature. The mixture was washed twice with NaHCO₃ solution and concentrated. The residue was chromatographed over silica gel.

### Variant II:

The piperazine derivative C was placed in 1% sodium hydroxide solution. Acetone was added until the mixture was monophasic. The residue B of the 1st stage was dissolved in acetone and added dropwise. The batch was concentrated, extracted twice with dichloromethane or ethyl acetate, and then concentrated. The residue was chromatographed over silica gel.

With regard to the following examples, it should be noted that the numbering of the examples does not necessarily correspond to the numbering of the compounds according to the preceding tables.

In the following ¹H NMR data, the comma as a decimal point is replaced by a period.

### Example 1: Synthesis of phenyl-[4-(4-phenylbutyl)piperazin-1-yl]methanone

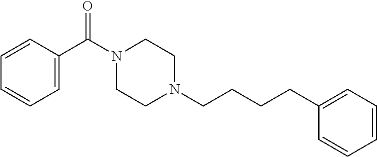 [SMILES: O=C(c1ccccc1)N1CCN(CCCCc2ccccc2)CC1]

### Method A:

¹H NMR (400 MHz, DMSO) δ 7.48-7.40 (m, 4H), 7.40-7.33 (m, 1H), 7.29-7.24 (m, 2H), 7.21-7.13 (m, 3H), 3.59 (s, 2H), 3.30 (s, 2H), 2.58 (t, J=7.6 Hz, 2H), 2.44-2.21 (m, 6H), 1.63-1.52 (m, 2H), 1.48-1.39 (m, 2H).

### Example 2: Synthesis of [4-[(E)-cinnamyl]piperazin-1-yl]-phenyl-methanone

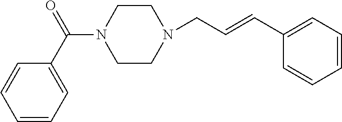 [SMILES: O=C(c1ccccc1)N1CCN(C/C=C/c2ccccc2)CC1]

### Method A:

¹H NMR (400 MHz, methanol-d4) δ 7.48-7.43 (m, 3H), 7.43-7.37 (m, 4H), 7.32-7.26 (m, 2H), 7.24-7.19 (m, 1H), 6.59 (d, J=15.8 Hz, 1H), 6.28 (dt, 1H), 3.79 (s, 2H), 3.47 (s, 2H), 3.21 (d, J=6.9 Hz, 1H), 2.61 (s, 2H), 2.49 (s, 2H).

### Example 3: Synthesis of phenyl-[4-(2-phenylethyl)piperazin-1-yl]methanone

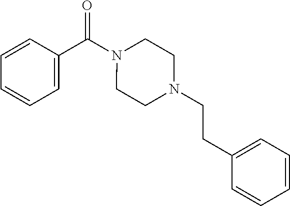 [SMILES: O=C(c1ccccc1)N1CCN(CCc2ccccc2)CC1]

### Method A:

¹H NMR (400 MHz, CDCl3) δ 7.40 (s, 5H), 7.32-7.24 (m, 2H), 7.23-7.17 (m, 3H), 3.92-3.74 (m, 2H), 3.46 (s, 2H), 2.81 (dd, J=9.9, 6.1 Hz, 2H), 2.69-2.61 (m, 2H), 2.61-2.35 (m, 4H).

### Example 4: Synthesis of [4-(2-cyclohexylethyl)piperazin-1-yl]-phenyl-methanone

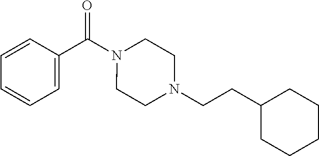 [SMILES: O=C(c1ccccc1)N1CCN(CCC2CCCCC2)CC1]

### Method A:

¹H NMR (400 MHz, CDCl3) δ 7.40 (s, 5H), 3.80 (s, 2H), 3.53-3.33 (m, 2H), 2.52 (d, J=9.0 Hz, 2H), 2.45-2.26 (m, 4H), 1.78-1.56 (m, 5H), 1.44-1.33 (m, 2H), 1.31-1.06 (m, 3H), 1.00-0.82 (m, 2H).

### Example 5: Synthesis of phenyl-[4-(3-phenylpropyl)-1-piperidyl]methanone

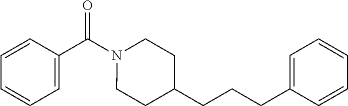 [SMILES: O=C(c1ccccc1)N1CCC(CCCc2ccccc2)CC1]

### Method A:

1H NMR (400 MHz, DMSO) δ 7.42, (dt, J=3.9, 2.7 Hz, 3H), 7.37-7.32 (m, 2H), 7.29-7.23 (m, 2H), 7.21-7.17 (m, 2H), 7.18-7.13 (m, 1H), 4.45 (s, 1H), 3.53 (s, 1H), 2.97 (s, 1H), 2.72 (s, 1H), 2.56 (t, J=7.7 Hz, 2H), 1.72 (s, 1H), 1.65-1.46 (m, 4H), 1.30-1.19 (m, 2H), 1.12-0.95 (m, 2H).

### Example 6: Synthesis of phenyl-[4-(3-phenylpropyl)piperazin-1-yl]methanone

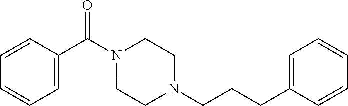 [SMILES: O=C(c1ccccc1)N1CCN(CCCc2ccccc2)CC1]

### Method A:

¹H NMR (400 MHz, CDCl3) δ 7.42-7.37 (m, 5H), 7.32-7.23 (m, 2H), 7.18 (td, J=5.7, 5.2, 2.8 Hz, 3H), 3.80 (s, 2H), 3.43 (s, 2H), 2.65 (t, J=7.7 Hz, 2H), 2.52 (s, 2H), 2.44-2.36 (m, 2H), 2.37 (s, 2H), 1.88-1.77 (m, 2H).

### Example 7: Synthesis of phenyl-[4-(2-phenylethyl)-1,4-diazepan-1-yl]methanone

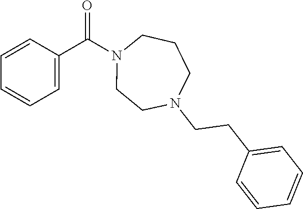 [SMILES: O=C(c1ccccc1)N1CCCN(CCc2ccccc2)CC1]

### Method A:

¹H NMR (400 MHz, DMSO) δ 7.45-7.39 (m, 3H), 7.38-7.30 (m, 2H), 7.30-7.20 (m, 3H), 7.20-7.12 (m, 2H), 3.66-3.57 (m, 2H), 3.40-3.25 (m, 2H), 2.83-2.77 (m, 2H), 2.77-2.59 (m, 6H), 1.88-1.76 (m, 2H), 1.72-1.60 (m, 2H).

### Example 8: Synthesis of (4-benzylpiperazin-1-yl)-phenyl-methanone

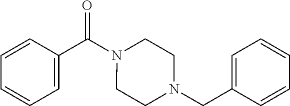 [SMILES: O=C(c1ccccc1)N1CCN(Cc2ccccc2)CC1]

### Method A:

¹H NMR (400 MHz, CDCl3) δ 7.39 (s, 5H), 7.32 (d, J=4.0 Hz, 4H), 7.30-7.21 (m, 1H), 3.79 (s, 2H), 3.54 (s, 2H), 3.42 (s, 2H), 2.53 (s, 2H), 2.38 (s, 2H).

### Example 9: Synthesis of [4-(2-cyclohexylethyl)piperazin-1-yl]-(3,4-diethoxyphenyl)methanone

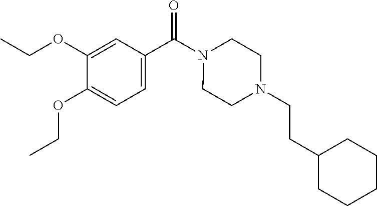 [SMILES: CCOc1ccc(C(=O)N2CCN(CCC3CCCCC3)CC2)cc1OCC]

### Method A:

¹H NMR (400 MHz, CDCl3) δ 6.98 (d, J=1.9 Hz, 1H), 6.95 (dd, J=8.1, 1.9 Hz, 1H), 6.85 (d, J=8.2 Hz, 1H), 4.11 (q, J=7.0 Hz, 2H), 4.10 (q, J=7.0 Hz, 2H), 3.82-3.42 (m, 4H), 2.51-2.41 (m, 4H), 2.38 (dd, J=9.1, 6.7 Hz, 2H), 1.76-1.58 (m, 5H), 1.46 (t, J=7.0 Hz, 3H), 1.45 (t, J=7.0 Hz, 3H), 1.44-1.33 (m, 2H), 1.32-1.07 (m, 4H), 0.91 (q, J=11.2, 10.6 Hz, 2H).

### Example 10: Synthesis of (3,4-diethoxyphenyl)-[4-(3-phenylpropyl)-1-piperidyl]methanone

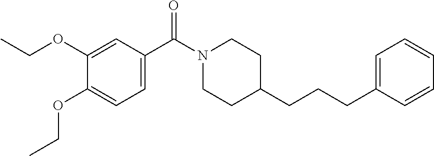 [SMILES: CCOc1ccc(C(=O)N2CCC(CCCc3ccccc3)CC2)cc1OCC]

### Method A:

¹H NMR (400 MHz, CDCl3) δ 7.32-7.23 (m, 2H), 7.20-7.14 (m, 3H), 6.95 (d, J=1.9 Hz, 1H), 6.93 (dd, J=8.1, 2.0 Hz, 1H), 6.84 (d, J=8.1 Hz, 1H), 4.60 (s, 2H), 4.14-4.06 (m, 4H), 2.83 (s, 2H), 2.60 (t, J=7.7 Hz, 2H), 1.91-1.59 (m, 4H), 1.53 (ddt, J=11.1, 7.5, 4.0 Hz, 1H), 1.45 (t, J=7.0 Hz, 3H), 1.44 (t, J=7.0 Hz, 3H), 1.36-1.26 (m, 2H), 1.15 (s, 2H).

### Example 11: Synthesis of (3,4-diethoxyphenyl)-[4-(2-phenylethyl)-1,4-diazepan-1-yl]methanone

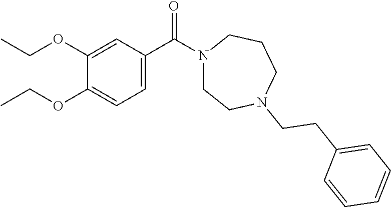 [SMILES: CCOc1ccc(C(=O)N2CCCN(CCc3ccccc3)CC2)cc1OCC]

### Method A:

¹H NMR (400 MHz, chloroform-d) δ 7.32-7.24 (m, 2H), 7.23-7.12 (m, 3H), 6.99-6.90 (m, 2H), 6.87-6.83 (m, 1H), 4.16-4.03 (m, 4H), 3.83-3.72 (m, 2H), 3.60-3.46 (m, 2H), 2.94-2.83 (m, 1H), 2.83-2.66 (m, 9H), 1.90 (d, J=59.6 Hz, 2H), 1.50-1.41 (m, 6H).

### Example 12: Synthesis of (3,4-dimethoxyphenyl)-[4-(4-phenylbutyl)piperazin-1-yl]methanone

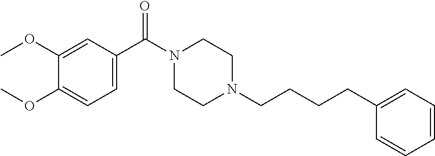 [SMILES: COc1ccc(C(=O)N2CCN(CCCCc3ccccc3)CC2)cc1OC]

### Method A:

¹H NMR (400 MHz, CDCl3) δ 7.31-7.24 (m, 2H), 7.21-7.14 (m, 3H), 7.01-6.95 (m, 2H), 6.85 (d, J=8.1 Hz, 1H), 3.90 (s, 3H), 3.90 (s, 3H), 3.86-3.20 (m, 2H), 2.63 (t, J=7.5 Hz, 2H), 2.59-2.23 (m, 6H), 2.24-1.76 (m, 2H), 1.74-1.60 (m, 2H), 1.60-1.39 (m, 2H).

### Example 13: Synthesis of (3,4-dimethoxyphenyl)-[4-(2-phenylethyl)piperazin-1-yl]methanone

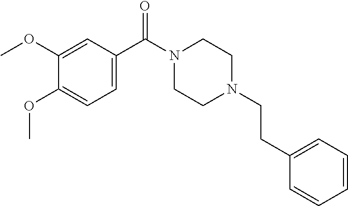 [SMILES: COc1ccc(C(=O)N2CCN(CCc3ccccc3)CC2)cc1OC]

### Method A:

¹H NMR (400 MHz, chloroform-d) δ 7.32-7.26 (m, 2H), 7.23-7.18 (m, 3H), 7.01-6.97 (m, 2H), 6.86 (d, J=8.1 Hz, 1H), 3.92-3.88 (m, 6H), 3.68 (s, 4H), 2.85-2.79 (m, 2H), 2.69-2.63 (m, 2H), 2.63-2.45 (m, 4H).

### Example 14: Synthesis of [4-(2-cyclohexylethyl)piperazin-1-yl]-(3,4-dimethoxyphenyl)methanone

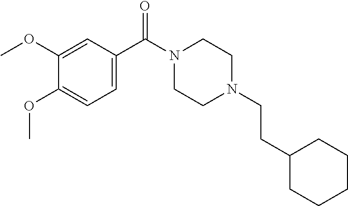 [SMILES: COc1ccc(C(=O)N2CCN(CCC3CCCCC3)CC2)cc1OC]

### Method A:

¹H NMR (400 MHz, CDCl3) δ 7.00 (d, J=1.8 Hz, 1H), 6.98 (dd, J=8.1, 1.9 Hz, 1H), 6.86 (d, J=8.1 Hz, 1H), 3.90 (s, 3H), 3.90 (s, 3H), 3.64 (s, 4H), 2.52-2.41 (m, 4H), 2.38 (dd, J=9.1, 6.7 Hz, 2H), 1.75-1.60 (m, 5H), 1.43-1.35 (m, 2H), 1.33-1.08 (m, 4H), 0.92 (q, J=11.1, 10.4 Hz, 2H).

### Example 15: Synthesis of (3,4-dimethoxyphenyl)-[4-(3-phenylpropyl)-1-piperidyl]methanone

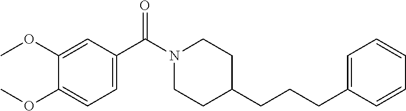 [SMILES: COc1ccc(C(=O)N2CCC(CCCc3ccccc3)CC2)cc1OC]

### Method A:

¹H NMR (400 MHz, CDCl3) δ 7.31-7.24 (m, 2H), 7.21-7.14 (m, 3H), 6.99-6.93 (m, 2H), 6.84 (d, J=8.0 Hz, 1H), 4.97-4.23 (m, 2H), 3.90 (s, 3H), 3.89 (s, 3H), 3.19-2.66 (m, 2H), 2.61 (t, J=7.7 Hz, 2H), 1.87-1.60 (m, 4H), 1.60-1.46 (m, 1H), 1.38-1.28 (m, 2H), 1.27-1.01 (m, 2H).

### Example 16: Synthesis of (3,4-dimethoxyphenyl)-[4-(3-phenylpropyl)piperazin-1-yl]methanone

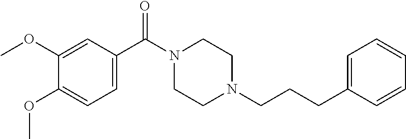 [SMILES: COc1ccc(C(=O)N2CCN(CCCc3ccccc3)CC2)cc1OC]

### Method A:

¹H NMR (400 MHz, chloroform-d) δ 7.30-7.25 (m, 2H), 7.21-7.16 (m, 3H), 7.00-6.95 (m, 2H), 6.85 (d, J=8.1 Hz, 1H), 3.90 (s, 3H), 3.89 (s, 3H), 3.84-3.35 (m, 4H), 2.65 (t, J=7.7 Hz, 2H), 2.42 (d, J=20.6 Hz, 4H), 2.44-2.36 (m, 2H), 1.83 (p, J=7.6 Hz, 2H).

### Example 17: Synthesis of (3,4-dimethoxyphenyl)-[4-(2-phenylethyl)-1,4-diazepan-1-yl]methanone

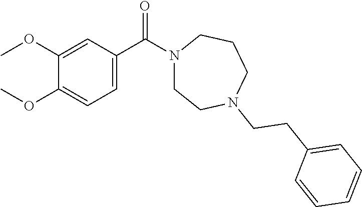 [SMILES: COc1ccc(C(=O)N2CCCN(CCc3ccccc3)CC2)cc1OC]

### Method A:

¹H NMR (400 MHz, CDCl3) δ 7.32-7.23 (m, 4H), 7.19 (dd, J=12.7, 5.5 Hz, 6H), 6.97 (d, J=7.4 Hz, 4H), 6.85 (d, J=8.6 Hz, 2H), 3.90 (s, 6H), 3.89-3.84 (m, 6H), 3.77 (d, J=9.6 Hz, 4H), 3.60-3.48 (m, 4H), 2.95-2.65 (m, 16H), 1.99 (s, 2H), 1.84 (s, 2H).

### Example 18: Synthesis of 4-benzylpiperazin-1-yl)-(3,4-dimethoxy-phenyl)methanone

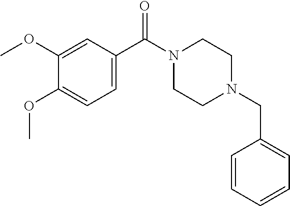 [SMILES: COc1ccc(C(=O)N2CCN(Cc3ccccc3)CC2)cc1OC]

### Method A:

¹H NMR (400 MHz, DMSO) δ 7.37-7.27 (m, 4H), 7.27-7.19 (m, 1H), 6.94 (d, J=8.2 Hz, 1H), 6.91 (d, J=1.4 Hz, 1H), 6.89 (dd, J=8.1, 1.8 Hz, 1H), 4.10-3.96 (m, 4H), 3.59-3.38 (m, 6H), 2.37 (s, 4H), 1.33 (t, J=6.9 Hz, 3H), 1.32 (t, J=6.9 Hz, 3H).

### Example 19: Synthesis of (4-methoxyphenyl)-[4-(4-phenylbutyl)piperazin-1-yl]methanone

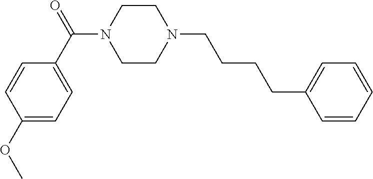 [SMILES: COc1ccc(C(=O)N2CCN(CCCCc3ccccc3)CC2)cc1]

### Method A:

¹H NMR (400 MHz, CDCl3) δ 7.41-7.35 (m, 2H), 7.31-7.23 (m, 2H), 7.21-7.13 (m, 3H), 6.94-6.87 (m, 2H), 3.83 (s, 3H), 3.93-3.36 (m, 4H), 2.63 (t, J=7.5 Hz, 2H), 2.44 (s, 4H), 2.42-2.34 (m, 2H), 1.72-1.59 (m, 2H), 1.59-1.45 (m, 2H).

### Example 20: Synthesis of (4-methoxyphenyl)-[4-(2-phenylethyl)piperazin-1-yl]methanone

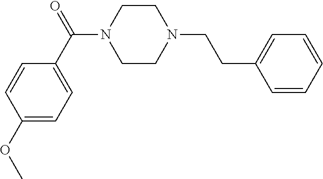 [SMILES: COc1ccc(C(=O)N2CCN(CCc3ccccc3)CC2)cc1]

### Method A:

¹H NMR (400 MHz, chloroform-d) δ 7.42-7.36 (m, 2H), 7.32-7.26 (m, 2H), 7.23-7.17 (m, 3H), 6.94-6.88 (m, 2H), 3.82 (s, 3H), 3.78-3.53 (m, 4H), 2.81 (dd, J=10.1, 6.0 Hz, 2H), 2.66-2.61 (m, 2H), 2.57-2.50 (m, 4H).

### Example 21: Synthesis of [4-(2-cyclohexylethyl)piperazin-1-yl]-(4-methoxyphenyl)methanone

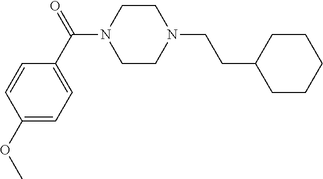 [SMILES: COc1ccc(C(=O)N2CCN(CCC3CCCCC3)CC2)cc1]

### Method A:

¹H NMR (400 MHz, CDCl3) δ 7.42-7.34 (m, 2H), 6.94-6.87 (m, 2H), 3.83 (s, 3H), 3.79-3.43 (m, 4H), 2.58-2.41 (m, 4H), 2.41-2.34 (m, 2H), 1.77-1.59 (m, 5H), 1.43-1.34 (m, 2H), 1.33-1.07 (m, 4H), 0.92 (qd, J=10.9, 10.0, 4.8 Hz, 2H).

### Example 22: Synthesis of (4-methoxyphenyl)-[4-(3-phenylpropyl)-1-piperidyl]-methanone

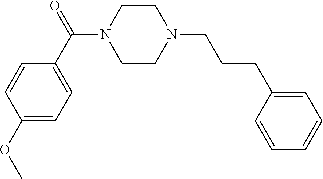 [SMILES: COc1ccc(C(=O)N2CCN(CCCc3ccccc3)CC2)cc1]

### Method A:

¹H NMR (400 MHz, CDCl3) δ 7.39-7.32 (m, 2H), 7.31-7.24 (m, 2H), 7.21-7.13 (m, 3H), 6.92-6.86 (m, 2H), 5.03-4.19 (m, 2H), 3.82 (s, 3H), 2.84 (s, 2H), 2.60 (t, J=7.7 Hz, 2H), 1.90-1.58 (m, 4H), 1.58-1.46 (m, 1H), 1.37-1.27 (m, 2H), 1.27-1.01 (m, 2H).

### Example 23: Synthesis of (4-methoxyphenyl)-[4-(3-phenylpropyl)-piperazin-1-yl]methanone

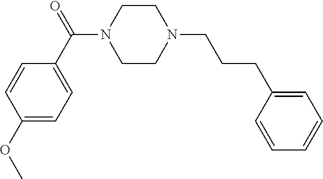 [SMILES: COc1ccc(C(=O)N2CCN(CCCc3ccccc3)CC2)cc1]

### Method A:

¹H NMR (400 MHz, chloroform-d) δ 7.40-7.35 (m, 2H), 7.30-7.25 (m, 2H), 7.21-7.15 (m, 3H), 6.93-6.88 (m, 2H), 3.82 (s, 3H), 3.77-3.32 (m, 4H), 2.65 (t, 2H), 2.52-2.42 (m, 4H), 2.42-2.37 (m, 2H), 1.87-1.78 (m, 2H).

### Example 24: Synthesis of (4-methoxyphenyl)-[4-(2-phenylethyl)-1,4-diazepan-1-yl]methanone

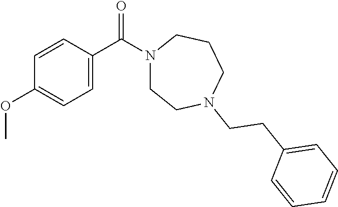 [SMILES: COc1ccc(C(=O)N2CCCN(CCc3ccccc3)CC2)cc1]

### Method A:

¹H NMR (400 MHz, chloroform-d) δ 7.40-7.32 (m, 2H), 7.30-7.25 (m, 2H), 7.24-7.12 (m, 3H), 6.90 (d, J=8.7 Hz, 2H), 3.82 (d, J=1.4 Hz, 3H), 3.81-3.65 (m, 2H), 3.61-3.43 (m, 2H), 2.97-2.65 (m, 8H), 2.04-1.90 (m, 2H), 1.90-1.75 (m, 2H).

### Example 25: Synthesis of (4-benzylpiperazin-1-yl)-(4-methoxyphenyl)methanone

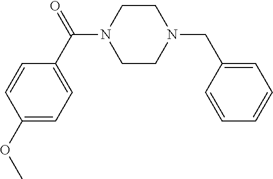 [SMILES: COc1ccc(C(=O)N2CCN(Cc3ccccc3)CC2)cc1]

### Method A:

¹H NMR (400 MHz, chloroform-d) δ 7.39-7.35 (m, 2H), 7.34-7.30 (m, 4H), 7.32-7.22 (m, 1H), 6.92-6.87 (m, 2H), 3.82 (s, 3H), 3.97-3.35 (m, 4H), 3.54 (s, 2H), 2.65-2.23 (m, 4H).

### Example 26: Synthesis of (4-ethoxyphenyl)-[4-(4-phenylbutyl)piperazin-1-yl]methanone

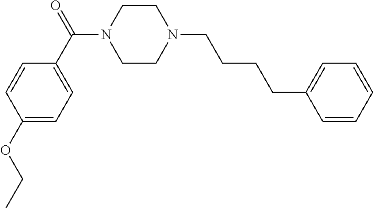 [SMILES: CCOc1ccc(C(=O)N2CCN(CCCCc3ccccc3)CC2)cc1]

### Method A:

¹H NMR (400 MHz, CDCl3) δ 7.38-7.33 (m, 2H), 7.30-7.23 (m, 2H), 7.20-7.14 (m, 3H), 6.92-6.85 (m, 2H), 4.05 (q, J=7.0 Hz, 2H), 3.61 (s, 4H), 2.63 (t, J=7.5 Hz, 2H), 2.50-2.40 (m, 4H), 2.40-2.33 (m, 2H), 1.71-1.59 (m, 2H), 1.53 (tt, J=8.8, 5.9 Hz, 2H), 1.42 (t, J=7.0 Hz, 3H).

### Example 27: Synthesis of [4-[(E)-cinnamyl]piperazin-1-yl]-(4-ethoxyphenyl)methanone

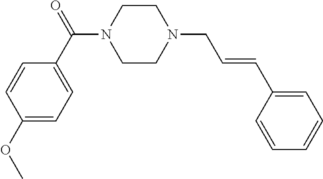 [SMILES: COc1ccc(C(=O)N2CCN(C/C=C/c3ccccc3)CC2)cc1]

### Method A:

¹H NMR (400 MHz, chloroform-d) δ 7.40-7.35 (m, 4H), 7.34-7.28 (m, 2H), 7.26-7.21 (m, 1H), 6.95-6.84 (m, 2H), 6.53 (dt, J=15.8, 1.0 Hz, 1H), 6.25 (dt, J=15.8, 6.8 Hz, 1H), 4.05 (q, J=7.0 Hz, 2H), 3.93-3.41 (m, 4H), 3.19 (dd, J=6.8, 1.4 Hz, 2H), 2.64-2.43 (m, 4H), 1.42 (t, J=7.0 Hz, 3H).

### Example 28: Synthesis of (4-ethoxyphenyl)-[4-(2-phenylethyl)piperazin-1-yl]methanone

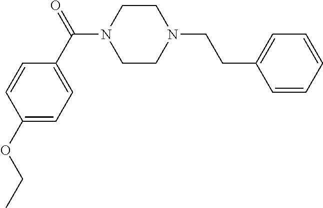 [SMILES: CCOc1ccc(C(=O)N2CCN(CCc3ccccc3)CC2)cc1]

### Method A:

1H NMR (400 MHz, CDCl3) δ 7.41-7.34 (m, 2H), 7.32-7.25 (m, 2H), 7.19 (dt, J=7.0, 2.9 Hz, 3H), 6.92-6.86 (m, 2H), 4.04 (q, J=7.0 Hz, 2H), 3.87-3.40 (m, 4H), 2.80 (dd, J=9.9, 6.2 Hz, 2H), 2.63 (dd, J=9.8, 6.3 Hz, 2H), 2.59-2.39 (m, 4H), 1.42 (t, J=7.0 Hz, 3H).

### Example 29: Synthesis of [4-(2-cyclohexylethyl)piperazin-1-yl]-(4-ethoxyphenyl)methanone

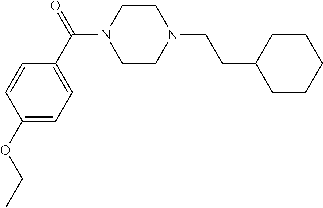 [SMILES: CCOc1ccc(C(=O)N2CCN(CCC3CCCCC3)CC2)cc1]

### Method A:

¹H NMR (400 MHz, CDCl3) δ 7.41-7.32 (m, 2H), 6.93-6.85 (m, 2H), 4.05 (q, J=7.0 Hz, 2H), 3.87-3.37 (m, 4H), 2.44 (s, 4H), 2.42-2.30 (m, 2H), 1.75-1.60 (m, 5H), 1.42 (t, J=7.0 Hz, 3H), 1.41-1.31 (m, 2H), 1.31-1.25 (m, 1H), 1.25-1.08 (m, 3H), 0.98-0.84 (m, 2H).

### Example 30: Synthesis of (4-ethoxyphenyl)-[4-(3-phenylpropyl)-1-piperidyl]methanone

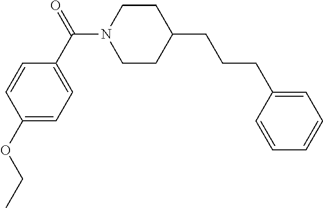 [SMILES: CCOc1ccc(C(=O)N2CCC(CCCc3ccccc3)CC2)cc1]

### Method A:

¹H NMR (400 MHz, DMSO) δ 7.30 (d, J=8.7 Hz, 2H), 7.32-7.22 (m, 2H), 7.22-7.17 (m, 2H), 7.17-7.09 (m, 1H), 6.97-6.91 (m, 2H), 4.05 (q, J=7.0 Hz, 2H), 3.03-2.67 (m, 4H), 2.56 (t, J=7.7 Hz, 2H), 1.72-1.62 (m, 2H), 1.65-1.53 (m, 2H), 1.50 (ddq, J=9.1, 5.8, 2.9, 2.5 Hz, 1H), 1.33 (t, J=7.0 Hz, 3H), 1.24 (q, J=7.2 Hz, 2H), 1.10-0.95 (m, 2H).

### Example 31: Synthesis of (4-ethoxyphenyl)-[4-(3-phenylpropyl)-piperazin-1-yl]methanone

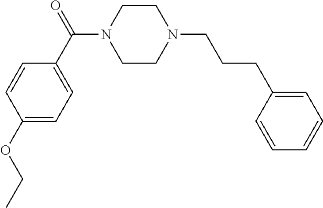 [SMILES: CCOc1ccc(C(=O)N2CCN(CCCc3ccccc3)CC2)cc1]

### Method A:

¹H NMR (400 MHz, chloroform-d) δ 7.39-7.33 (m, 2H), 7.30-7.24 (m, 2H), 7.20-7.14 (m, 3H), 6.90-6.85 (m, 2H), 4.04 (q, J=7.0 Hz, 2H), 3.94-3.29 (m, 4H), 2.64 (t, J=7.7 Hz, 2H), 2.48-2.34 (m, 4H), 2.38 (t, 2H), 1.82 (p, J=7.6, 7.2 Hz, 2H), 1.41 (t, J=7.0 Hz, 3H).

### Example 32: Synthesis of (4-ethoxyphenyl)-[4-(2-phenylethyl)-1,4-diazepan-1-yl]methanone

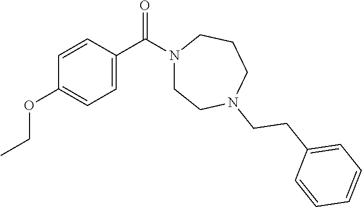 [SMILES: CCOc1ccc(C(=O)N2CCCN(CCc3ccccc3)CC2)cc1]

### Method B:

¹H NMR (400 MHz, CDCl3) δ 7.39-7.31 (m, 2H), 7.31-7.23 (m, 2H), 7.23-7.13 (m, 3H), 6.91-6.85 (m, 2H), 4.05 (q, J=7.0 Hz, 2H), 3.83-3.65 (m, 2H), 3.59-3.44 (m, 2H), 2.95-2.62 (m, 8H), 2.05-1.89 (m, 2H), 1.89-1.75 (m, 2H), 1.42 (t, J=7.0 Hz, 3H).

### Example 33: Synthesis of (4-benzylpiperazin-1-yl)-(4-ethoxyphenyl)methanone

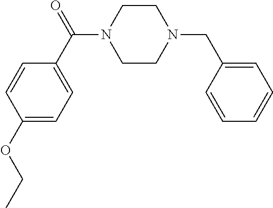 [SMILES: CCOc1ccc(C(=O)N2CCN(Cc3ccccc3)CC2)cc1]

### Method B:

¹H NMR (400 MHz, CDCl3) δ 7.39-7.34 (m, 2H), 7.33-7.28 (m, 4H), 7.29-7.23 (m, 1H), 6.91-6.85 (m, 2H), 4.05 (q, J=7.0 Hz, 2H), 3.89-3.37 (m, 4H), 3.54 (s, 2H), 2.60-2.28 (m, 4H), 1.42 (t, J=7.0 Hz, 3H).

### Example 34: Synthesis of [4-[(E)-cinnamyl]piperazin-1-yl]-(3,4-diethoxy-phenyl)-methanone

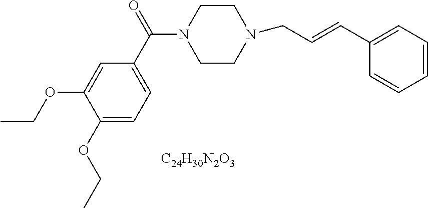 [SMILES: CCOc1ccc(C(=O)N2CCN(C/C=C/c3ccccc3)CC2)cc1OCC ; "C24H30N2O3"]

### Method B:

1H NMR (400 MHz, chloroform-d) δ 7.40-7.35 (m, 2H), 7.34-7.28 (m, 2H), 7.26-7.21 (m, 1H), 6.98 (d, J=1.9 Hz, 1H), 6.95 (dd, J=8.1, 2.0 Hz, 1H), 6.85 (d, J=8.2 Hz, 1H), 6.53 (d, J=15.8 Hz, 1H), 6.25 (dt, J=15.8, 6.7 Hz, 1H), 4.10 (qd, J=7.0, 2.7 Hz, 4H), 3.65 (m, 4H), 3.19 (dd, J=6.8, 1.3 Hz, 2H), 2.51 (ms, 4H), 1.45 (td, J=7.0, 2.2 Hz, 6H).

### Example 35: Synthesis of (3,4-diethoxyphenyl)-[4-(4-phenylbutyl)piperazin-1-yl]methanone

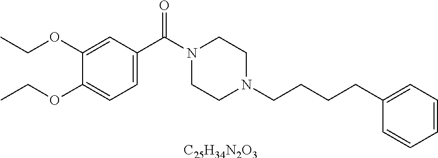 [SMILES: CCOc1ccc(C(=O)N2CCN(CCCCc3ccccc3)CC2)cc1OCC ; "C25H34N2O3"]

### Method B:

¹H NMR (400 MHz, chloroform-d) δ 7.30-7.24 (m, 2H), 7.20-7.14 (m, 3H), 6.97 (d, J=1.9 Hz, 1H), 6.94 (dd, J=8.2, 2.0 Hz, 1H), 6.85 (d, J=8.2 Hz, 1H), 4.10 (q, J=7.0, 4.4 Hz, 4H), 3.62 (s, 4H), 2.63 (t, J=7.5 Hz, 2H), 2.53-2.29 (m, 6H), 1.65 (q, 2H), 1.54 (q, 2H), 1.45 (t, J=7.0, 3.1 Hz, 6H).

### Example 36: Synthesis of (3,4-diethoxyphenyl)-[4-[2-(4-pyridyl)ethyl]piperazin-1-yl]methanone

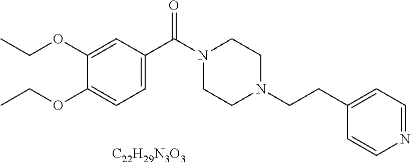 [SMILES: CCOc1ccc(C(=O)N2CCN(CCc3ccncc3)CC2)cc1OCC ; "C22H29N3O3"]

### Method B:

¹H NMR (400 MHz, chloroform-d) δ 8.54-8.36 (m, 2H), 7.33-7.20 (m, 2H), 7.05-6.84 (m, 3H), 4.14-3.93 (m, 4H), 2.82-2.52 (m, 4H), 2.68-2.30 (m, 8H), 1.41-1.26 (m, 6H).

### Example 37: Synthesis of (3,4-diethoxyphenyl)-[4-(2-phenoxyethyl)piperazin-1-yl]methanone

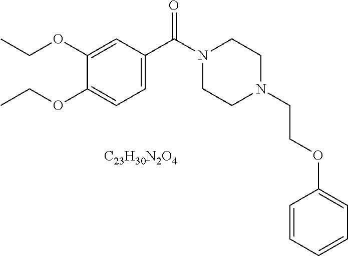 [SMILES: CCOc1ccc(C(=O)N2CCN(CCOc3ccccc3)CC2)cc1OCC ; "C23H30N2O4"]

### Method B:

¹H NMR (400 MHz, chloroform-d) δ 7.28 (m, 2H), 6.98 (d, J=8.22, 1H), 6.93 (m, 5H), 4.08 (t, J=5.93, 2H), 4.06 (m, 2H), 4.03 (m, 2H), 3.47 (m, 4H), 2.74 (t, J=5.71, 2H), 2.51 (m, 4H), 1.34 (t, J=7.11, 3H), 1.32 (t, J=7.14, 3H).

### Sensory Tasting of the Active Ingredients According to the Invention

The sensory tasting of the active ingredients according to the invention was carried out as follows:
(1) The active ingredients were each prepared in a 5% sugar solution or in an unflavored standard fondant mass. Fondant is the name for a soft, pasty sugar mass that is used to make various confectionery products. Fondant is made from water and sucrose as well as glucose syrup, invert sugar cream and/or sugar alcohols (e.g., sorbitol or mannitol). The mixture is boiled and then processed into a soft paste by kneading it vigorously and cooling it quickly.
(2) The concentration of the active ingredients was 25 or 50 ppm.
(3) The test samples were tested under practical conditions by a panel of experts with sensory training. For tasting, 3 to 7 tasters each rinsed their mouths with approx. 5 ml of the sugar solution and spat the solution out again. The mouth-watering and/or tingling effect was assessed on a scale of 1 (very weak) to 10 (very strong) and the profile evaluated.

The results of the sensory tests are summarized in Table 15 below:

TABLE 15

| Comp. | | Chemical | Dosage | Sensory |
|---|---|---|---|---|
| No. | Structure | Designation | ppm | Rating |
| 1 | 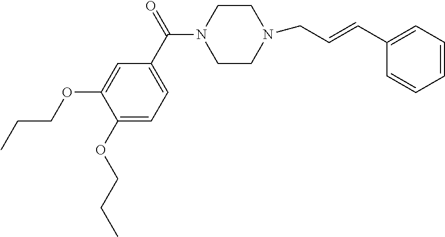 [SMILES: CCCOc1ccc(C(=O)N2CCN(C/C=C/c3ccccc3)CC2)cc1OCCC] | [4-[(E)-cinnamyl] pi-perazin-1-yl]-(3,4- dipropoxy- phenyl)methanone | 25 | astringent mouth-watering, Spilanthes effect |
| 2 | 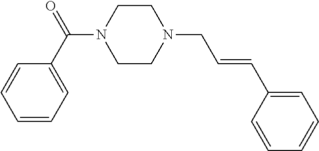 [SMILES: O=C(c1ccccc1)N1CCN(C/C=C/c2ccccc2)CC1] | 1,3-benzodioxol-5- yl-[4-[(E)-cinnamyl]- piperazin-1- yl]methanone | 50 | tingling/ fizzing, balsamic |
| 3 | 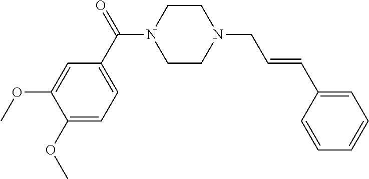 [SMILES: COc1ccc(C(=O)N2CCN(C/C=C/c3ccccc3)CC2)cc1OC] | [4-[(E)-cinnamyl] piperazin-1-yl]-(3,4- dimethoxy- phenyl)methanone | 50 | tingling/ fizzing |
| 4 | 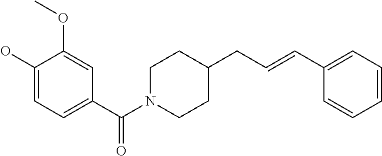 [SMILES: COc1cc(C(=O)N2CCC(C/C=C/c3ccccc3)CC2)ccc1O] | [4-[(E)-cinnamyl] piperazin-1-yl]-(4- hydroxy-3-methoxy- phenyl)methanone | 50 | balsamic |
| 5 | 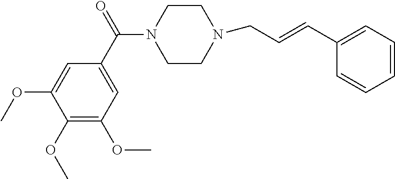 [SMILES: COc1cc(C(=O)N2CCN(C/C=C/c3ccccc3)CC2)cc(OC)c1OC] | [4-[(E)-cinnamyl] piperazin-1-yl]- (3,4,5-trimethoxy- phenyl)methanone | 25 | chocolaty, dusty, after cress |
| 6 | 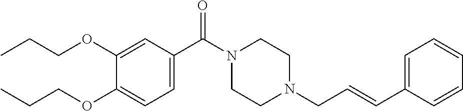 [SMILES: CCCOc1ccc(C(=O)N2CCN(C/C=C/c3ccccc3)CC2)cc1OCCC] | [4-[(E)-cinnamyl] piperazin-1-yl]-(3,4- dipropoxy- phenyl)methanone | 25 | bitter, light astringent |
| 7 | 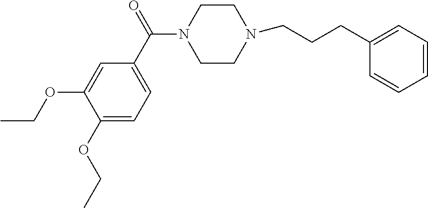 [SMILES: CCOc1ccc(C(=O)N2CCN(CCCc3ccccc3)CC2)cc1OCC] | (3,4-Diethoxy- phenyl)-[4-(3- phenyl- propyl)piperazin- 1-yl]methanone | 25 | tingling/ fizzing, mouth-watering, slightly cooling |
| 8 | 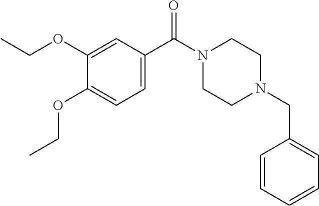 [SMILES: CCOc1ccc(C(=O)N2CCN(Cc3ccccc3)CC2)cc1OCC] | (4-benzylpiperazin- 1-yl)-(3,4-diethoxy- phenyl)methanone | 25 | slightly tingling/ fizzing, slightly mouth-watering, slightly cooling |
| 9 | 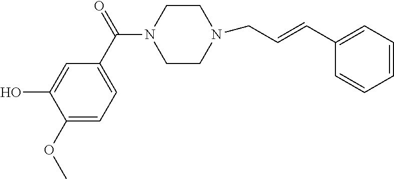 [SMILES: COc1ccc(C(=O)N2CCN(C/C=C/c3ccccc3)CC2)cc1O] | [4-[(E)-cinnamyl] piperazin-1-yl]-(3- hydroxy-4-methoxy- phenyl)methanone | 25 | sweet, after melon |
| 10 | 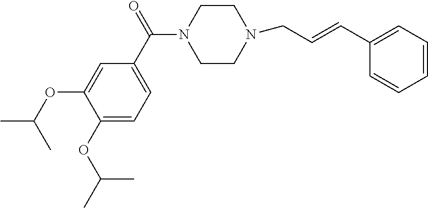 [SMILES: CC(C)Oc1ccc(C(=O)N2CCN(C/C=C/c3ccccc3)CC2)cc1OC(C)C] | [4-[(E)-cinnamyl] piperazin-1-yl]-(3,4- diisopropoxy- phenyl)methanone | 25 | astringent, bitter |
| 11 | 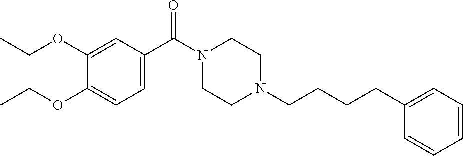 [SMILES: CCOc1ccc(C(=O)N2CCN(CCCCc3ccccc3)CC2)cc1OCC] | (3,4-Diethoxy- phenyl)-[4-(4-phenyl- butyl)piperazin- 1-yl]methanone | 25 | tingling/ fizzing, like melon, astringent, mouth-watering |
| 12 | 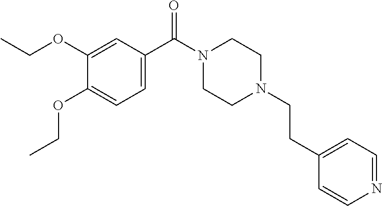 [SMILES: CCOc1ccc(C(=O)N2CCN(CCc3ccncc3)CC2)cc1OCC] | (3,4-Diethoxy- phenyl)-[4-[2-(4- pyridyl)ethyl] piperazin-1-yl] methanone | 25 | tingling/ fizzing, mouth-watering, ethanolic |
| 13 | 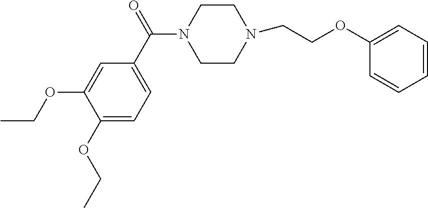 [SMILES: CCOc1ccc(C(=O)N2CCN(CCOc3ccccc3)CC2)cc1OCC] | (3,4-Diethoxy- phenyl)-[4-(2- phenoxyethyl) piperazin-1-yl] methanone | 25 | strong tingling/ fizzing, mouth-watering, |
| 14 | 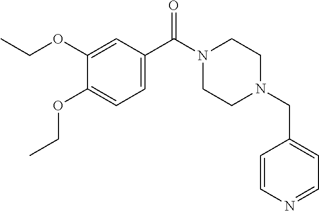 [SMILES: CCOc1ccc(C(=O)N2CCN(Cc3ccncc3)CC2)cc1OCC] | (3,4-Diethoxy- phenyl)-[4-(4- pyridyl- methyl)piperazin- 1-yl]methanone | 25 | slightly tingling/ fizzing, mouth-watering, burning, |
| 15 | 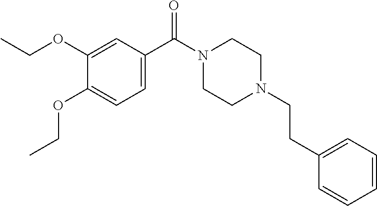 [SMILES: CCOc1ccc(C(=O)N2CCN(CCc3ccccc3)CC2)cc1OCC] | (3,4-Diethoxy- phenyl)-[4-(2-phenyl- ethyl)piperazin- 1-yl]methanone | 25 | Acid effect, tingling/ fizzing, mouth-watering, slightly salty |
| 16 | 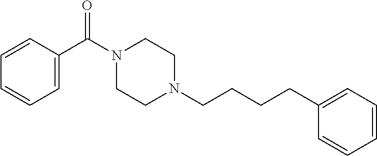 [SMILES: O=C(c1ccccc1)N1CCN(CCCCc2ccccc2)CC1] | Phenyl-[4-(4- phenylbutyl) piperazin- 1-yl]methanone | 25 and 50 | (25 ppm) slightly tingling/ fizzing; (50 ppm) metallic taste, mouth-watering and tingling/ fizzing effect (2-3) |
| 17 | 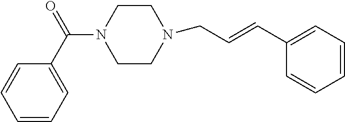 [SMILES: O=C(c1ccccc1)N1CCN(C/C=C/c2ccccc2)CC1] | [4-[(E)-cinnamyl] piperazin-1-yl]- phenyl-methanone | 50 | slightly tingling/ fizzing, slightly honey-like (3) |
| 18 | 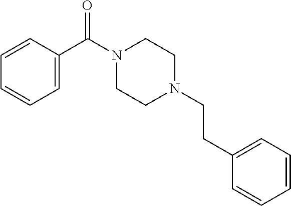 [SMILES: O=C(c1ccccc1)N1CCN(CCc2ccccc2)CC1] | Phenyl-[4-(2-phenyl- ethyl)piperazin- 1-yl]methanone | 25 and 50 | (25 ppm) no effect; 50 ppm) slightly tingling/ fizzing and mouth-watering (2-3) |
| 19 | 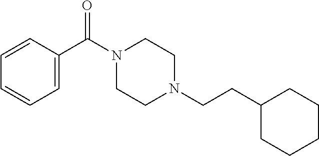 [SMILES: O=C(c1ccccc1)N1CCN(CCC2CCCCC2)CC1] | [4-(2-cyclo- hexylethyl)piperazin- 1-yl]-phenyl- methanone | 25 and 50 | (25 ppm) fruity, slightly mouth-watering; (50 ppm) of mushrooms, fruity, slightly metallic taste, slightly tingling/ fizzing (1-2) |
| 20 | 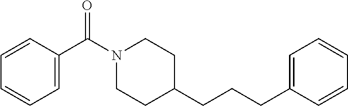 [SMILES: O=C(c1ccccc1)N1CCC(CCCc2ccccc2)CC1] | Phenyl-[4-(3-phenyl- propyl)-1-piperidyl]- methanone | 50 | slightly tingling/ fizzing (2), mouth-watering, Anthranilate- like, fruity note |
| 21 | 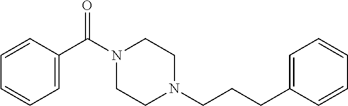 [SMILES: O=C(c1ccccc1)N1CCN(CCCc2ccccc2)CC1] | Phenyl-[4-(3- phenylpropyl) piperazin-1-yl] methanone | 25 and 50 | (25 ppm) sweet, fruity, slightly tingling/ fizzing; (50 ppm) slightly tingling/ fizzing and mouth-watering (2-3) |
| 22 | 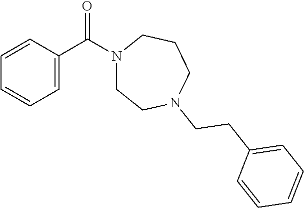 [SMILES: O=C(c1ccccc1)N1CCCN(CCc2ccccc2)CC1] | Phenyl-[4-(2-phenyl- ethyl)-1,4- diazepan-1-yl] methanone | 25 and 50 | (25 ppm) floral, linalool flavor; (50 ppm) very light metallic taste, remarkably mouth-watering and tingling/ fizzing (4-5) |
| 23 | 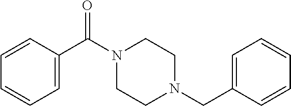 [SMILES: O=C(c1ccccc1)N1CCN(Cc2ccccc2)CC1] | (4-benzylpiperazin- 1-yl)-phenyl- methanone | 50 | Slightly floral taste, slightly metallic, not a pleasant taste, very slightly mouth-watering and tingling/fizzing (3) |
| 24 | 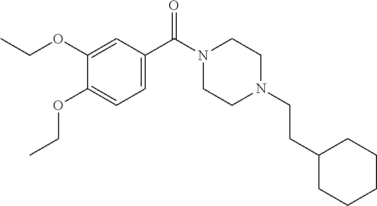 [SMILES: CCOc1ccc(C(=O)N2CCN(CCC3CCCCC3)CC2)cc1OCC] | [4-(2-cyclo- hexylethyl) piperazin-1-yl]- (3,4-diethoxy- phenyl)methanone | 50 | fruity taste (blueberries) |
| 25 | 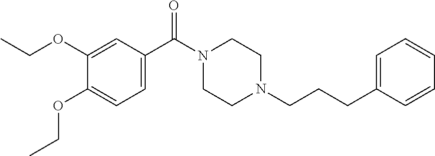 [SMILES: CCOc1ccc(C(=O)N2CCN(CCCc3ccccc3)CC2)cc1OCC] | (3,4-Diethoxy- phenyl)-[4-(3-phenyl- propyl)-1-piperidyl]- methanone | 50 | Very slightly tingling/fizzing (2-3), sharp, astringent |
| 26 | 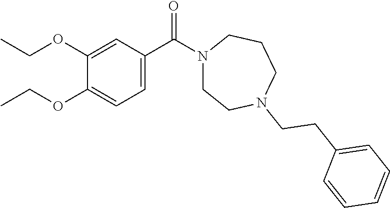 [SMILES: CCOc1ccc(C(=O)N2CCCN(CCc3ccccc3)CC2)cc1OCC] | (3,4-Diethoxy- phenyl)-[4-(2-phenyl- ethyl)-1,4-diazepan- 1-yl]methanone | 50 | Very slightly tingling/fizzing (1-2), and mouth-watering (weak) |
| 27 | 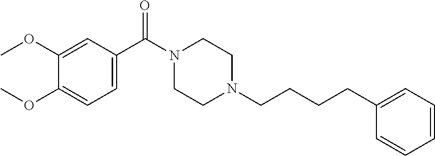 [SMILES: COc1ccc(C(=O)N2CCN(CCCCc3ccccc3)CC2)cc1OC] | (3,4-Dimethoxy- phenyl)-[4-(4-phenyl- butyl)piperazin- 1-yl]methanone | 50 | slightly tingling/ fizzing (4-5), and mouth-watering, metallic after- taste, weakly burning |
| 28 | 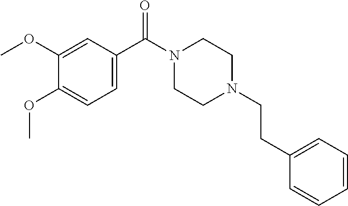 [SMILES: COc1ccc(C(=O)N2CCN(CCc3ccccc3)CC2)cc1OC] | (3,4-Dimethoxy- phenyl)-[4-(2-phenyl- ethyl)piperazin- 1-yl]methanone | 25 and 50 | slightly tingling/ fizzing (3) slightly metallic |
| 29 | 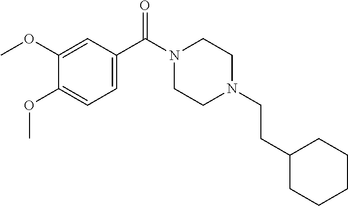 [SMILES: COc1ccc(C(=O)N2CCN(CCC3CCCCC3)CC2)cc1OC] | [4-(2-cyclo- hexylethyl) piperazin- 1-yl]-(3,4- dimethoxy- phenyl)methanone | 50 | slightly mouth-watering, slightly tingling/ fizzing (1-2), green note, light onion flavor |
| 30 | 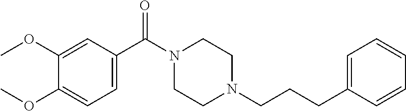 [SMILES: COc1ccc(C(=O)N2CCN(CCCc3ccccc3)CC2)cc1OC] | (3,4-Dimethoxy- phenyl)-[4-(3-phenyl- propyl)-1- piperidyl]methanone | 50 | very slight tingling/fizzing and mouth-watering, dry in the mouth |
| 31 | 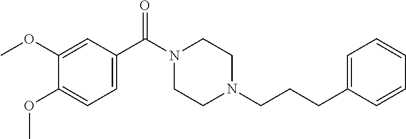 [SMILES: COc1ccc(C(=O)N2CCN(CCCc3ccccc3)CC2)cc1OC] | (3,4-Dimethoxy- phenyl)-[4-(3-phenyl- propyl)piperazin- 1-yl]methanone | 50 | tingling/ fizzing (5-6), and mouth-watering, fruity note, Honey flavor |
| 32 | 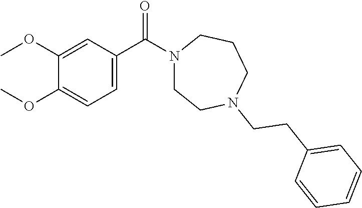 [SMILES: COc1ccc(C(=O)N2CCCN(CCc3ccccc3)CC2)cc1OC] | (3,4-Dimethoxy- phenyl)-[4-(2-phenyl- ethyl)-1,4-diazepan- 1-yl]methanone | 50 | very slightly tingling/ fizzing (1), slightly metallic taste |
| 33 | 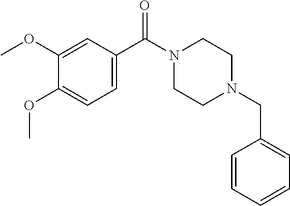 [SMILES: COc1ccc(C(=O)N2CCN(Cc3ccccc3)CC2)cc1OC] | (4-benzylpiperazin- 1-yl)-(3,4- dimethoxy- phenyl)methanone | 50 | very slightly tingling/ fizzing (1-2), slightly like marzipan |
| 34 | 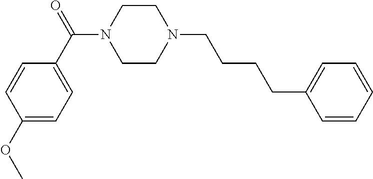 [SMILES: COc1ccc(C(=O)N2CCN(CCCCc3ccccc3)CC2)cc1] | (4-Methoxy- phenyl)-[4-(4-phenyl- butyl)piperazin- 1-yl]methanone | 25 and 50 | (25 ppm) tingling/ fizzing and mouth-watering effect, neutral taste; (50 ppm) strongly mouth-watering/ tingling/fizzing (6) |
| 35 | 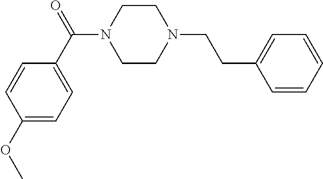 [SMILES: COc1ccc(C(=O)N2CCN(CCc3ccccc3)CC2)cc1] | (4-Methoxy- phenyl)-[4-(2-phenyl- ethyl)piperazin- 1-yl]methanone | 25 and 50 | (25 ppm) slightly mouth-watering; (50 ppm) slightly tingling/ fizzing (3) and mouth-watering, Acid taste, Honey note |
| 36 | 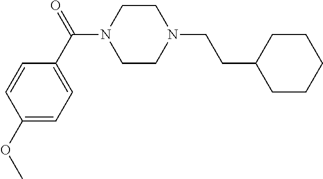 [SMILES: COc1ccc(C(=O)N2CCN(CCC3CCCCC3)CC2)cc1] | [4-(2-cyclo- hexylethyl)piperazin- 1-yl]-(4-methoxy- phenyl) methanone | 50 | slightly tingling/fizzing (3), metallic taste |
| 37 | 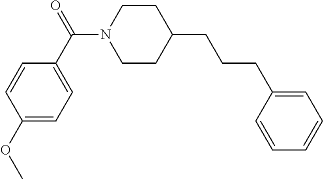 [SMILES: COc1ccc(C(=O)N2CCC(CCCc3ccccc3)CC2)cc1] | (4-Methoxy- phenyl)-[4-(3-phenyl- propyl)-1- piperidyl]methanone | 50 | slightly tingling/ fizzing (2), light fruity note |
| 38 | 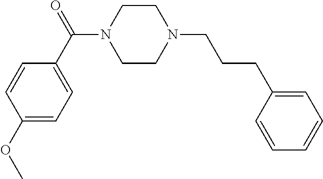 [SMILES: COc1ccc(C(=O)N2CCN(CCCc3ccccc3)CC2)cc1] | (4-Methoxy- phenyl)-[4-(3- phenylpropyl) piperazin-1-yl] methanone | 25 and 50 | (25 ppm) slightly mouth-watering, anesthetic; (50 ppm), remarkably tingling/fizzing (6), numbing, Increasing effect over time |
| 39 | 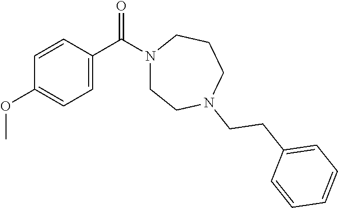 [SMILES: COc1ccc(C(=O)N2CCCN(CCc3ccccc3)CC2)cc1] | (4-ethoxyphenyl)- [4-(2-phenylethyl)- 1,4-diazepan-1- yl]methanone | 50 | slightly tingling/ fizzing (4), slightly metallic taste |
| 40 | 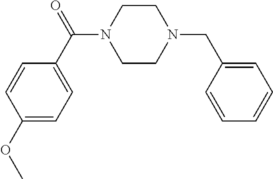 [SMILES: COc1ccc(C(=O)N2CCN(Cc3ccccc3)CC2)cc1] | (4-benzylpiperazin- 1-yl)-(4-methoxy- phenyl)methanone | 50 | mouth-watering, neutral, slightly tingling/ fizzing (1-2) |
| 41 | 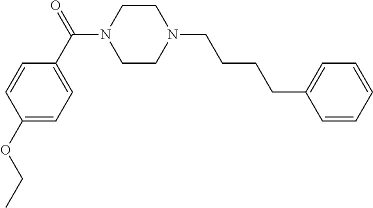 [SMILES: CCOc1ccc(C(=O)N2CCN(CCCCc3ccccc3)CC2)cc1] | (4-ethoxyphenyl)- [4-(4-phenyl- butyl)piperazin-1- yl]methanone | 25 and 50 | (25 ppm) metallic, mouth- watering effect; (50 ppm) strong mouth- watering effect (8), long-lasting, Slightly hot |
| 42 | 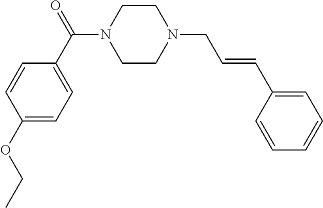 [SMILES: CCOc1ccc(C(=O)N2CCN(C/C=C/c3ccccc3)CC2)cc1] | [4-[(E)-cinnamyl] piperazin-1-yl]-(4- ethoxy- phenyl)methanone | 50 | slightly tingling (3-4), and mouth- watering, fruity, creamy, Vanilla flavor |
| 43 | 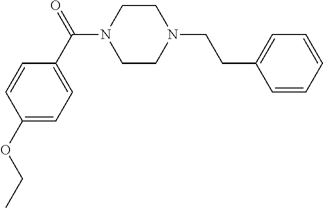 [SMILES: CCOc1ccc(C(=O)N2CCN(CCc3ccccc3)CC2)cc1] | (4-ethoxyphenyl)- [4-(2- phenylethyl) piperazin- 1-yl]methanone | 25 and 50 | (25 ppm) metallic, slightly numbing, tingling/fizzing and mouth- watering effect; (50 ppm) strongly bitter, metallic, tingling/fizzing (7) , and mouth-watering effect |
| 44 | 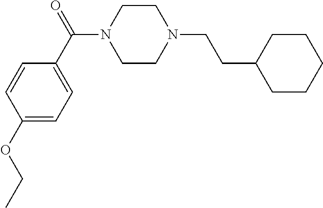 [SMILES: CCOc1ccc(C(=O)N2CCN(CCC3CCCCC3)CC2)cc1] | [4-(2-cyclo- hexylethyl) piperazin-1-yl]- (4-ethoxy- phenyl)methanone | 50 | slightly tingling/fizzing (4), and mouth-watering |
| 45 | 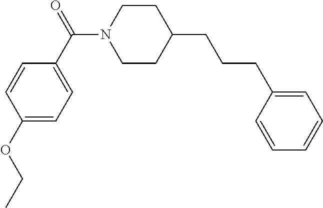 [SMILES: CCOc1ccc(C(=O)N2CCC(CCCc3ccccc3)CC2)cc1] | (4-ethoxyphenyl)- [4-(3-phenyl- propyl)-1-piperidyl]- methanone | 50 | slightly tin- gling/ fizzing (2) |
| 46 | 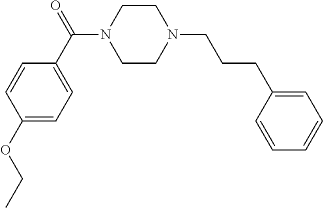 [SMILES: CCOc1ccc(C(=O)N2CCN(CCCc3ccccc3)CC2)cc1] | (4-ethoxyphenyl)- [4-(3-phenyl- propyl)piperazin-1- yl]methanone | 25 and 50 | (25 ppm) metallic, slightly numbing, tingling and mouth- watering effect; (50 ppm) strong, bitter, metallic and tingling/fizzing (7), and mouth-watering effect |
| 47 | 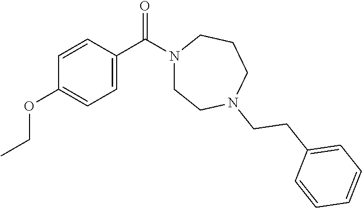 [SMILES: CCOc1ccc(C(=O)N2CCCN(CCc3ccccc3)CC2)cc1] | (4-ethoxyphenyl)- [4-(2-phenylethyl)- 1,4-diazepan-1- yl]methanone | 50 | Strong mouth-watering and tingling/fizzing effect (6), Pleasant taste |
| 48 | 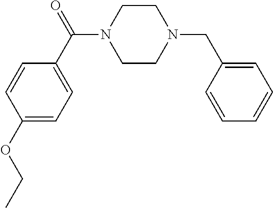 [SMILES: CCOc1ccc(C(=O)N2CCN(Cc3ccccc3)CC2)cc1] | (4-benzylpiperazin- 1-yl)-(4-ethoxy- phenyl)methanone | 50 | slightly tingling/ fizzing (1-2) |

As can be seen from the tasting results of the compounds according to Table 15, all active ingredients according to the invention showed a mouth-watering and/or tingling/fizzing effect with significantly few side effects, such as a bitter or metallic or astringent aftertaste.

A pronounced mouth-watering or tingling/fizzing effect was observed with almost all tested compounds No. 1, 2, 3, 7, 8, 11, 12, 14, 15, 16, 17, 18, 19, 20, 21, 22, 23, 25, 26, 27, 28, 29, 30, 31, 32, 33, 34, 35, 36, 37, 38, 39, 40, 41, 42, 43, 44, 45, 46, 47, and 48 according to Table 15.

Of the samples tested, compounds no. 19, 20, 26, 29, 32, 33, 37, 40 and 48 in particular showed a mouth-watering, tingling/fizzing effect according to Table 15, which was rated with an average intensity of 1 to 2.

Of the samples tested, compounds No. 16, 17, 18, 21, 23, 25, 28, 35, 36, 39, 42 or 44 in particular showed a mouth-watering, tingling/fizzing effect according to Table 15, which was rated with an average intensity of 3 to 4.

Compounds No. 22, 27 and 39 according to Table 15 showed a mouth-watering, tingling/fizzing effect, which was rated with an average intensity of 4 to 5.

According to Table 15, compounds no. 31, 34, 38 and 47 had an even more intensive mouth-watering, tingling/fizzing effect with an average intensity of 5 to 6.

Compounds 41, 43 and 46 according to Table 15 showed the most intense mouth-watering, tingling/fizzing effect with an average intensity of 7 to 8. Compound 41 had a strong mouth-watering effect at a concentration of 50 ppm, which lasted for a long time. Compound 47 according to Table 15 had a strong mouth-watering and tingling/fizzing effect with a pleasant taste at a concentration of 50 ppm.

### FORMULATION EXAMPLES

The following application examples 1.1 to 1.4 and formulation examples 1.5 to 1.21 show a wide variety of formulations for food and cosmetic preparations.

In these examples, the comma as a decimal point is replaced by a period.

Unless otherwise stated, all figures below are in % by weight.

### Example of Use 1.1: Typical Pungent Aroma Compositions Containing the Active Ingredients According to the Invention

TABLE 16

| Component | A | B | C | D | E | F | G | H |
|---|---|---|---|---|---|---|---|---|
| 10% by weight pellitorin in 1,2- | 0.25 | 0.25 | 0.25 | 0.25 | 0.25 | 0.25 | 0.25 | 0.25 |
| propylene glycol/diethyl | | | | | | | | |
| Hesperetin | 2.50 | 2.50 | 2.50 | 2.50 | 2.50 | 2.50 | 2.50 | 2.50 |
| Phloretin | 1.50 | 1.50 | 1.50 | 1.50 | 1.50 | 1.50 | 1.50 | 1.50 |
| (3,4-Diethoxyphenyl)-[4-(2-phe- | 1.50 | — | 1.00 | — | — | — | — | — |
| noxyethyl)piperazin-1-yl]meth- | | | | | | | | |
| anone (8/13) | | | | | | | | |
| (3,4-Diethoxyphenyl)-[4-(2-phe- | — | 0.10 | 0.05 | — | — | — | 0.10 | — |
| nylethyl)piperazin-1-yl]meth- | | | | | | | | |
| anone (23/15) | | | | | | | | |
| [4-(2-cyclohexylethyl)piperazin- | 0.05 | — | — | 0.50 | — | — | — | — |
| 1-yl]-phenyl-methanone (9/19) | | | | | | | | |
| Phenyl-[4-(2-phenylethyl)-1,4- | — | — | — | — | 0.05 | — | 0.40 | — |
| diazepan-1-yl]methanon | | | | | | | | |
| (4-Methoxyphenyl)-[4-(4-phe- | | — | — | — | 0.05 | 0.07 | — | — |
| nylbutyl)piperazin-1-yl]meth- | | | | | | | | |
| anone (13/34) | | | | | | | | |
| (4-ethoxyphenyl)-[4-(4-phenyl- | 0.05 | — | — | — | — | — | 0.10 | — |
| butyl)piperazin-1-yl]methanone | | | | | | | | |
| (15/41) | | | | | | | | |
| (4-ethoxyphenyl)-[4-(2-phe- | | — | — | — | — | — | — | 0.10 |
| nylethyl)piperazin-1-yl]meth- | | | | | | | | |
| anone (36/43) | | | | | | | | |
| Propylene glycol | ad 100 | ad 100 | ad 100 | ad 100 | ad 100 | ad 100 | ad 100 | ad 100 |

The ingredients (substances or solutions) are mixed in the proportions specified above and then absorbed with propylene glycol and completely dissolved by heating slightly.

### Example of Use 1.2: Spray-Dried Aroma Compositions Containing the Active Ingredients According to the Invention

TABLE 17

| Component | A | B | C | D | E | F | G |
|---|---|---|---|---|---|---|---|
| (3,4-Diethoxyphenyl)-[4-(2-phenoxy- | 9.00 | — | — | — | — | — | — |
| ethyl)piperazin-1-yl]methanone (8/13) | | | | | | | |
| [4-(2-cyclohexylethyl)piperazin-1-yl]- | 0.20 | 3.50 | — | — | — | — | — |
| phenyl-methanone (9/19) | | | | | | | |
| Phenyl-[4-(2-phenylethyl)-1,4-diaze- | — | — | 5.00 | — | 0.05 | \|0.10 | — |
| pan-1-yl]methanon (44/22) | | | | | | | |
| (4-Methoxyphenyl)-[4-(4-phenyl- | | — | — | 2.50 | — | — | — |
| butyl)piperazin-1-yl]methanone | | | | | | | |
| (13/34) | | | | | | | |
| (4-ethoxyphenyl)-[4-(4-phenylbutyl)pi- | — | — | — | — | 0.15 | 0.10 | — |
| perazin-1-yl]methanone (15/41) | | | | | | | |
| (4-ethoxyphenyl)-[4-(2-phenylethyl)pi- | 0.10 | | | | 0.10 | | |
| perazin-1-yl]methanone (36/43) | | | | | | | |
| (3,4-Dimethoxyphenyl)-[4-(3-phe- | — | — | — | — | — | — | 0.25 |
| nylpropyl)piperazin-1-yl]methanone | | | | | | | |
| (30/31) | | | | | | | |
| Maltodextrin | ad 100 | ad 100 | ad 100 | ad 100 | ad 100 | ad 100 | ad 100 |

The ingredients are dissolved in a mixture of ethanol and demineralized water and then spray-dried.

### Application Example 1.3: Preparation of Mouthwash Flavors Using the Active Ingredients According to the Invention

TABLE 18

| Component | A | B | C | D | E | F | G | H |
|---|---|---|---|---|---|---|---|---|
| Anethole | 30 | 30 | 30 | 30 | 30 | 30 | 30 | 30 |
| Eucalyptol | 25 | 25 | 25 | 25 | 25 | 25 | 25 | 25 |
| I-Menthol | 37.50 | 36.50 | 38.50 | 36.20 | 36.00 | 36.60 | 39.75 | 34.70 |
| Optacool A ® | — | 8 | — | 8 | — | 8 | — | 8 |
| Coolact 10 ® | 5 | — | 5 | — | 5 | — | 5 | — |
| (3,4- | 2.00 | — | — | — | 3.00 | — | — | 2.00 |
| Diethoxyphenyl)-[4- | | | | | | | | |
| (2-phenoxyethyl)- | | | | | | | | |
| piperazin-1- | | | | | | | | |
| yl]methanone (8/13) | | | | | | | | |
| [4-(2- | 0.10 | 0.50 | — | — | — | — | — | — |
| cyclohexylethyl)- | | | | | | | | |
| piperazin-1-yl]- | | | | | | | | |
| phenyl-methanone | | | | | | | | |
| (9/19) | | | | | | | | |
| Phenyl-[4-(2-phenyl- | — | — | 1.50 | — | 1.00 | — | — | — |
| ethyl)-1,4-diazepan- | | | | | | | | |
| 1-yl]methanon | | | | | | | | |
| (44/22) | | | | | | | | |
| (4-Methoxyphenyl)- | — | — | — | 0.80 | — | — | 0.10 | — |
| [4-(4-phenylbutyl)- | | | | | | | | |
| piperazin-1- | | | | | | | | |
| yl]methanone | | | | | | | | |
| (13/34) | | | | | | | | |
| (4-ethoxyphenyl)-[4- | — | — | — | — | — | 0.15 | — | — |
| (4-phenylbutyl)- | | | | | | | | |
| piperazin-1- | | | | | | | | |
| yl]methanone | | | | | | | | |
| (15/41) | | | | | | | | |
| (4-ethoxyphenyl)-[4- | 0.40 | — | — | — | — | 0.20 | 0.15 | — |
| (2-phenylethyl)- | | | | | | | | |
| piperazin-1- | | | | | | | | |
| yl]methanone | | | | | | | | |
| (36/43) | | | | | | | | |
| (3,4-Dimethoxyphe- | — | — | — | — | — | 0.05 | — | 0.30 |
| nyl)-[4-(3-phenyl- | | | | | | | | |
| propyl)-piperazin-1- | | | | | | | | |
| yl]methanone | | | | | | | | |
| (30/31) | | | | | | | | |
| Total | 100 | 100 | 100 | 100 | 100 | 100 | 100 | 100 |

### Application Example 1.4: Preparation of Peppermint-Flavored Flavors Using Substances According to the Invention

TABLE 19

| Component | A | B | C | D | E | F | G | H | I |
|---|---|---|---|---|---|---|---|---|---|
| Peppermint oil | ad 100 | ad 100 | ad 100 | ad 100 | ad 100 | ad 100 | ad 100 | ad 100 | ad 100 |
| Mentha arvensis | | | | | | | | | |
| I-Menthon | 5 | 5 | 5 | 5 | 5 | 5 | 5 | 5 | 5 |
| I-Menthol | 5 | 5 | 5 | 5 | 5 | 5 | 5 | 5 | 5 |
| (3,4-Diethoxyphenyl)- | 1.00 | — | — | — | 1.00 | — | — | 0.50 | — |
| [4-(2-phenoxyethyl)- | | | | | | | | | |
| piperazin-1- | | | | | | | | | |
| yl]methanone (8/13) | | | | | | | | | |
| [4-(2-cyclohexylethyl)- | — | 0.50 | — | — | — | 0.30 | — | — | 0.50 |
| piperazin-1-yl]-phenyl- | | | | | | | | | |
| methanone (9/19) | | | | | | | | | |
| Phenyl-[4-(2-phenyl- | — | — | 0.75 | — | 1.00 | 0.30 | 0.05 | — | — |
| ethyl)-1,4-diazepan-1- | | | | | | | | | |
| yl]methanon (44/22) | | | | | | | | | |
| (4-Methoxyphenyl)-[4- | 0.05 | — | — | 1.20 | — | — | — | 0.05 | — |
| (4- | | | | | | | | | |
| phenylbutyl)piperazin- | | | | | | | | | |
| 1-yl]methanone (13/34) | | | | | | | | | |
| (4-ethoxyphenyl)-[4-(4- | — | — | — | — | — | — | 0.05 | — | — |
| phenylbutyl)piperazin- | | | | | | | | | |
| 1-yl]methanone (15/41) | | | | | | | | | |
| (4-ethoxyphenyl)-[4-(2- | 0.10 | | | | | | 0.10 | 0.20 | |
| phenylethyl)piperazin- | | | | | | | | | |
| 1-yl]methanone (36/43) | | | | | | | | | |
| (3,4-Dimethoxyphenyl)- | — | — | — | — | — | — | — | — | 0.30 |
| [4-(3-phenylpropyl)- | | | | | | | | | |
| piperazin-1- | | | | | | | | | |
| yl]methanone (30/31) | | | | | | | | | |
| Total | 100 | 100 | 100 | 100 | 100 | 100 | 100 | 100 | 100 |

The aroma compositions listed above were used in the application examples described below.

### Example 1.5: Alcohol-Reduced Drink—Production of a Beer Mix Drink with Reduced Alcohol Content or without Alcohol

TABLE 20

| Component | A* | B | C | D | E | F | G | H | I |
|---|---|---|---|---|---|---|---|---|---|
| Sugar syrup | 4 | 4 | 4 | 4 | 4 | 4 | 4 | 4 | 4 |
| Beer (4.9% vol.) | 50 | — | — | — | — | — | — | — | — |
| Beer (non-alcoholic | — | — | 50 | — | 50 | — | 50 | — | 50 |
| and | | | | | | | | | |
| calorie-reduced, | | | | | | | | | |
| 2.8% vol.) | | | | | | | | | |
| Beer (non-alcoholic, | — | 50 | — | 50 | — | 50 | — | 50 | — |
| 0%) | | | | | | | | | |
| Citric acid | 0.15 | 0.15 | 0.15 | 0.15 | 0.15 | 0.15 | 0.15 | 0.15 | 0.15 |
| Ascorbic acid | 0.01 | 0.01 | 0.01 | 0.01 | 0.01 | 0.01 | 0.01 | 0.01 | 0.01 |
| Grapefruit juice | 6 | 6 | 6 | 6 | 6 | 6 | 6 | 6 | 6 |
| Grapefruit flavor | 0.20 | 0.20 | 0.20 | 0.20 | 0.20 | 0.20 | 0.20 | 0.20 | 0.20 |
| Aroma composition | — | 0.20 | 0.20 | — | — | — | — | — | — |
| (application | | | | | | | | | |
| example 1.1A) | | | | | | | | | |
| Aroma composition | — | — | — | 0.20 | 0.20 | — | — | — | — |
| (application | | | | | | | | | |
| example 1.1B) | | | | | | | | | |
| Aroma composition | — | — | — | — | — | 0.20 | 0.20 | — | — |
| (application | | | | | | | | | |
| example 1.1E) | | | | | | | | | |
| Aroma composition | — | — | — | — | — | — | — | 0.20 | 0.20 |
| (application | | | | | | | | | |
| example 1.1H) | | | | | | | | | |
| Water | ad 100 | ad 100 | ad 100 | ad 100 | ad 100 | ad 100 | ad 100 | ad 100 | ad 100 |
| Carbonic acid | 0.70 | 0.70 | 0.70 | 0.70 | 0.70 | 0.70 | 0.70 | 0.70 | 0.70 |

Note:
* not according to the invention

### Example 1.6: Applications in a Toothpaste (‘Silica Opaque’)

TABLE 21

| Component | A | B | C | D | E | F | G | H | I | K |
|---|---|---|---|---|---|---|---|---|---|---|
| Deionized water | 26.53 | 26.53 | 26.53 | 26.53 | 26.53 | 26.53 | 26.53 | 26.53 | 26.53 | 26.53 |
| Sorbitol 70% | 45 | 44.975 | 45 | 44.975 | 45 | 44.975 | 45 | 44.975 | 45 | 44.975 |
| Solbrol M Na salt | 0.15 | 0.15 | 0.15 | 0.15 | 0.15 | 0.15 | 0.15 | 0.15 | 0.15 | 0.15 |
| Trisodium phosphate | 0.10 | 0.10 | 0.10 | 0.10 | 0.10 | 0.10 | 0.10 | 0.10 | 0.10 | 0.10 |
| Saccharin | 0.20 | 0.20 | 0.20 | 0.20 | 0.20 | 0.20 | 0.20 | 0.20 | 0.20 | 0.20 |
| Sodium | 1.12 | 1.12 | 1.12 | 1.12 | 1.12 | 1.12 | 1.12 | 1.12 | 1.12 | 1.12 |
| monofluorophosphate | | | | | | | | | | |
| PEG 1500 | 5 | 5 | 5 | 5 | 5 | 5 | 5 | 5 | 5 | 5 |
| Sident 9 (Abrasive | 10 | 10 | 10 | 10 | 10 | 10 | 10 | 10 | 10 | 10 |
| Silica) | | | | | | | | | | |
| Sident 22 S (thickening | 8 | 8 | 8 | 8 | 8 | 8 | 8 | 8 | 8 | 8 |
| silica) | | | | | | | | | | |
| Sodium carboxymethyl | 0.90 | 0.90 | 0.90 | 0.90 | 0.90 | 0.90 | 0.90 | 0.90 | 0.90 | 0.90 |
| cellulose | | | | | | | | | | |
| Titanium (IV) oxide | 0.50 | 0.50 | 0.50 | 0.50 | 0.50 | 0.50 | 0.50 | 0.50 | 0.50 | 0.50 |
| Sodium lauryl sulfate | 1.50 | 1.50 | 1.50 | 1.50 | 1.50 | 1.50 | 1.50 | 1.50 | 1.50 | 1.50 |
| (SLS) | | | | | | | | | | |
| Pellitorin solution PLM | — | 0.025 | — | 0.025 | — | 0.025 | — | 0.025 | — | 0.025 |
| (containing 10% | | | | | | | | | | |
| pellitorin) | | | | | | | | | | |
| Aroma | 1.00 | 1.00 | — | — | — | — | — | — | — | — |
| Peppermint type | | | | | | | | | | |
| (Application | | | | | | | | | | |
| example 1.4A) | | | | | | | | | | |
| Aroma | — | — | 1.00 | 1.00 | — | — | — | — | — | — |
| Peppermint type | | | | | | | | | | |
| (Application | | | | | | | | | | |
| example 1.4B) | | | | | | | | | | |
| Aroma | — | — | — | — | 1.00 | 1.00 | — | — | — | — |
| Peppermint type | | | | | | | | | | |
| (Application | | | | | | | | | | |
| example 1.4C) | | | | | | | | | | |
| Aroma | — | — | — | — | — | — | 1.00 | 1.00 | — | — |
| Peppermint type | | | | | | | | | | |
| (Application | | | | | | | | | | |
| example 1.4G) | | | | | | | | | | |
| Aroma | — | — | — | — | — | — | — | — | 1.00 | 1.00 |
| Peppermint type | | | | | | | | | | |
| (Application | | | | | | | | | | |
| example 1.4H) | | | | | | | | | | |
| Total | 100 | 100 | 100 | 100 | 100 | 100 | 100 | 100 | 100 | 100 |

### Example 1.7: Application in a Toothpaste (Calcium Carbonate Base)

TABLE 22

| Component | A | B | C | D | E | F | G | H | I | K |
|---|---|---|---|---|---|---|---|---|---|---|
| Deionized water | 27.5 | 27.48 | 27.5 | 27.48 | 27.5 | 27.48 | 26.5 | 26.48 | 27.5 | 27.48 |
| Saccharin | 0.20 | 0.20 | 0.20 | 0.20 | 0.20 | 0.20 | 0.20 | 0.20 | 0.20 | 0.20 |
| Solbrol M sodium | 0.20 | 0.20 | 0.20 | 0.20 | 0.20 | 0.20 | 0.20 | 0.20 | 0.20 | 0.20 |
| salt | | | | | | | | | | |
| Sodium | 0.80 | 0.80 | 0.80 | 0.80 | 0.80 | 0.80 | 0.80 | 0.80 | 0.80 | 0.80 |
| monofluorophosphate | | | | | | | | | | |
| Sorbitol 70% | 29 | 29 | 29 | 29 | 29 | 29 | 29 | 29 | 29 | 29 |
| Calcium carbonate | 35 | 35 | 35 | 35 | 35 | 35 | 35 | 35 | 35 | 35 |
| Sident 22 S | 2.50 | 2.50 | 2.50 | 2.50 | 2.50 | 2.50 | 2.50 | 2.50 | 2.50 | 2.50 |
| (thickening silica) | | | | | | | | | | |
| Sodium carboxy- | 1.30 | 1.30 | 1.30 | 1.30 | 1.30 | 1.30 | 1.30 | 1.30 | 1.30 | 1.30 |
| methylcellulose | | | | | | | | | | |
| Titanium dioxide | 0.50 | 0.50 | 0.50 | 0.50 | 0.50 | 0.50 | 0.50 | 0.50 | 0.50 | 0.50 |
| Sodium lauryl | 2 | 2 | 2 | 2 | 2 | 2 | 2 | 2 | 2 | 2 |
| sulfate | | | | | | | | | | |
| Pellitorin solution | — | 0.02 | — | 0.02 | — | 0.02 | — | 0.02 | — | 0.02 |
| PLM (containing | | | | | | | | | | |
| 10% pellitorin) | | | | | | | | | | |
| Aroma | 1.00 | 1.00 | — | — | — | — | — | — | — | — |
| Peppermint type | | | | | | | | | | |
| (Application | | | | | | | | | | |
| example 1.4D) | | | | | | | | | | |
| Aroma | — | — | 1.00 | 1.00 | — | — | — | — | — | — |
| Peppermint type | | | | | | | | | | |
| (Application | | | | | | | | | | |
| example 1.4E) | | | | | | | | | | |
| Aroma | — | — | — | — | 1.00 | 1.00 | — | — | — | — |
| Peppermint type | | | | | | | | | | |
| (Application | | | | | | | | | | |
| example 1.4F) | | | | | | | | | | |
| Aroma | — | — | — | — | — | — | 2.00 | 2.00 | — | — |
| Peppermint type | | | | | | | | | | |
| (Application | | | | | | | | | | |
| example 1.4H) | | | | | | | | | | |
| Aroma | — | — | — | — | — | — | — | — | 1.00 | 1.00 |
| Peppermint type | | | | | | | | | | |
| (Application | | | | | | | | | | |
| example 1.41) | | | | | | | | | | |
| Total | 100 | 100 | 100 | 100 | 100 | 100 | 100 | 100 | 100 | 100 |

### Example 1.8: Application in a Sugar-Free Chewing Gum

TABLE 23

| Part | Component | Weight % |
|---|---|---|
| A | Chewing gum base, Company “Jagum T” | 30.00 |
| B | Sorbitol, powdered | 39.00 |
| | Isomalt ® (Palatinit GmbH) | 9.50 |
| | Xylitol | 2.00 |
| | Mannitol | 3.00 |
| | Aspartame ® | 0.10 |
| | Acesulfame ® K | 0.10 |
| | Emulgum ® (Colloides Naturels, Inc.) | 0.30 |
| C | Sorbitol, 70% | 14.00 |
| | Glycerine | 1.00 |
| D | Aroma compositions according to application example | 1.00 |

Parts A to D are mixed and kneaded intensively. The resulting raw mass can then be processed into ready-to-eat chewing gum, e.g. in the form of thin strips.

### Example 1.9: Mouthwash (“Ready to Use” without Alcohol)

TABLE 24

| Component | A | B | C | D | E | F | G | H | I | K |
|---|---|---|---|---|---|---|---|---|---|---|
| Cremophor RH | 1.80 | 1.80 | 1.80 | 1.80 | 1.80 | 1.80 | 1.80 | 1.80 | 1.80 | 1.80 |
| 455 | | | | | | | | | | |
| Sorbitol 70% | 10 | 10 | 10 | 10 | 10 | 10 | 10 | 10 | 10 | 10 |
| Sodium fluoride | 0.18 | 0.18 | 0.18 | 0.18 | 0.18 | 0.18 | 0.18 | 0.18 | 0.18 | 0.18 |
| Sodium | 0.10 | 0.10 | 0.10 | 0.10 | 0.10 | 0.10 | 0.10 | 0.10 | 0.10 | 0.10 |
| saccharin 450 | | | | | | | | | | |
| Solbrol M sodium | 0.15 | 0.15 | 0.15 | 0.15 | 0.15 | 0.15 | 0.15 | 0.15 | 0.15 | 0.15 |
| salt | | | | | | | | | | |
| Pellitorin solution | — | 0.0125 | — | 0.0125 | — | 0.0125 | — | 0.0125 | — | 0.0125 |
| PLM (containing | | | | | | | | | | |
| 10% pellitorin) | | | | | | | | | | |
| Mouthwash flavor | 4.00 | 4.00 | — | — | — | — | — | — | — | — |
| (Application | | | | | | | | | | |
| example 1.3A) | | | | | | | | | | |
| Mouthwash flavor | — | — | 4.00 | 4.00 | — | — | — | — | — | — |
| (Application | | | | | | | | | | |
| example 1.3B) | | | | | | | | | | |
| Mouthwash flavor | — | — | — | — | 4.00 | 4.00 | — | — | — | — |
| (application | | | | | | | | | | |
| example 1.3C) | | | | | | | | | | |
| Mouthwash flavor | — | — | — | — | — | — | 4.00 | 4.00 | — | — |
| (Application | | | | | | | | | | |
| example 1.3F) | | | | | | | | | | |
| Mouthwash flavor | — | — | — | — | — | — | — | — | 4.00 | 4.00 |
| (Application | | | | | | | | | | |
| example 1.3H) | | | | | | | | | | |
| Deionized water | ad 100 | ad 100 | ad 100 | ad 100 | ad 100 | ad 100 | ad 100 | ad 100 | ad 100 | ad 100 |
| Total | 100 | 100 | 100 | 100 | 100 | 100 | 100 | 100 | 100 | 100 |

### Example 1.10: Toothpaste and Mouthwash as a 2-In-1 Product

TABLE 25

| Component | A | B | C | D | E | F | G |
|---|---|---|---|---|---|---|---|
| Ethanol, 96% | 5 | 5 | 5 | 5 | 5 | 5 | 5 |
| Sorbitol, 70% in water | 40 | 40 | 40 | 40 | 40 | 40 | 40 |
| Glycerine | 20 | 20 | 20 | 20 | 20 | 20 | 20 |
| Saccharin | 0.20 | 0.20 | 0.20 | 0.20 | 0.20 | 0.20 | 0.20 |
| Na monofluorophosphate | 0.76 | 0.76 | 0.76 | 0.76 | 0.76 | 0.76 | 0.76 |
| Solbrol M, Na salt | 0.15 | 0.15 | 0.15 | 0.15 | 0.15 | 0.15 | 0.15 |
| Abrasive silica (Sident 9) | 20 | 20 | 20 | 20 | 20 | 20 | 20 |
| Thickening silica (Sident 22S) | 2 | 2 | 2 | 2 | 2 | 2 | 2 |
| Na-carboxymethylcellulose | 0.30 | 0.30 | 0.30 | 0.30 | 0.30 | 0.30 | 0.30 |
| Sodium lauryl sulfate | 1.20 | 1.20 | 1.20 | 1.20 | 1.20 | 1.20 | 1.20 |
| Green dye (1% in water) | 0.50 | 0.50 | 0.50 | 0.50 | 0.50 | 0.50 | 0.50 |
| Aroma composition | 2.00 | — | — | — | — | — | — |
| (Application example 1.3A) | | | | | | | |
| Aroma composition | — | 2.00 | — | — | — | — | — |
| (Application example 1.3B) | | | | | | | |
| Aroma composition | — | — | 2.00 | — | — | — | — |
| (Application example 1.3C) | | | | | | | |
| Aroma composition | — | — | — | 2.00 | — | — | — |
| (Application example 1.3D) | | | | | | | |
| Aroma composition | — | — | — | — | 2.00 | — | — |
| (Application example 1.3E) | | | | | | | |
| Aroma composition | — | — | — | — | — | 2.00 | — |
| (Application example 1.3G) | | | | | | | |
| Aroma composition | — | — | — | — | — | — | 2.00 |
| (Application example 1.3H) | | | | | | | |
| Distilled water | ad 100 | ad 100 | ad 100 | ad 100 | ad 100 | ad 100 | ad 100 |
| Total | 100 | 100 | 100 | 100 | 100 | 100 | 100 |

### Example 1.11: Use in Combination with a Pungent Plant Extract as an Alcohol Booster

### Comparative Sample Liqueur Base 10% Vol

| | |
|---|---|
| 7.39 kg | Alcohol, p.a. goods |
| 20 kg | Invert sugar syrup, 66.5% dry matter |
| 72.61 kg | Water |
| Total 100 kg | |

### Liqueur Base 5.5% Vol.

| | |
|---|---|
| 4.06 kg | alcohol, p.a. goods |
| 20 kg | Invert sugar syrup, 66.5% dry matter |
| 75.94 kg | water |
| Total 100 kg | |

Version A: Liqueur base 5.5% vol+0.3% of a 10% solution of an extract of grains of paradise in ethanol
Version B: Liqueur base 5.5% vol+0.075% of a 10% solution of an extract of grains of paradise in ethanol+0.02% of a solution of (4-ethoxyphenyl)-[4-(4-phenylbutyl)piperazin-1-yl]methanone (36/43) 1% in ethanol (equivalent to 2 ppm)
Version C: Liqueur base 5.5% vol+0.075% of a 10% solution of an extract of grains of paradise in ethanol+0.015% of a solution of (4-ethoxyphenyl)-[4-(2-phenylethyl)piperazin-1-yl]methanone (37/46) 1% in ethanol (corresponds to 1.5 ppm)

In versions B and C, the alcoholic sharpness of the reference sample is better imitated than in version A. Version A and the reference sample are very similar in sensory terms.

### Example 1.12: Foot Balm

TABLE 26

| | Weight % | Ingredient (INCI) |
|---|---|---|
| A | 2.00 | Ceteareth-6, stearyl alcohol |
| | 2.00 | Ceteareth-25 |
| | 5.00 | Cetearyl ethyl hexanoate |
| | 4.00 | Cetyl alcohol |
| | 4.00 | Glyceryl stearate |
| | 5.00 | Mineral oil |
| | 0.20 | Menthol |
| | 0.50 | Camphor |
| B | 69.30 | Aqua dem. |
| | q.s. | Preservative |
| C | 1.00 | Bisabolol |
| | 1.00 | Tocopheryl acetate |
| D | 1.00 | aqueous solution with 1-1.5% (3,4-diethoxyphenyl)- |
| | | [4-(2-phenoxyethyl)piperazin-1-yl]methanone (8/13) |
| | 5.00 | Witch hazel extract |
| | 100 | Total |

Heat the components of phases A and B separately to approx. 80° C. Stir phase B into phase A while homogenizing. Cool to approx. 40° C. while stirring, add phases C and D and homogenize briefly. Cool to room temperature while stirring.

### Example 1.13: Sugar-Free Hard Caramels

TABLE 27

| Component | Weight % |
|---|---|
| Palatinite, type M | 75.47 |
| Water | 24.03 |
| Peppermint flavor | 0.10 |
| Aroma preparation (application example 1.2A) | 0.40 |
| Total | 100 |

Palatinite was first mixed with water. The mixture was then melted at 165° C. and then cooled to 115° C. The peppermint flavoring and the flavoring preparation (application example 1.2) were then added. After mixing, the mixtures were poured into molds, removed from the molds after solidification and then individually packaged.

### Example 1.14: Seasoning Mix, Type “Pepper”

A=comparative preparation
B, C, D, E, F, G=preparations according to the invention

TABLE 28

| Component | A* | B | C | D | E | F | G |
|---|---|---|---|---|---|---|---|
| Milk protein | 0.80 | 0.80 | 0.80 | 0.80 | 0.80 | 0.80 | 0.80 |
| Carob seed | 2.00 | 2.00 | 2.00 | 2.00 | 2.00 | 2.00 | 2.00 |
| flour | | | | | | | |
| Corn starch | 24.00 | 23.00 | 23.00 | 23.00 | 23.00 | 23.00 | 23.00 |
| Common | 14.00 | 14.00 | 14.00 | 14.00 | 14.00 | 14.00 | 14.00 |
| salt | | | | | | | |
| Paprika | 12.00 | 12.00 | 12.00 | 12.00 | 12.00 | 12.00 | 12.00 |
| powder | | | | | | | |
| Tomato | 12.00 | 12.00 | 12.00 | 12.00 | 12.00 | 12.00 | 12.00 |
| powder | | | | | | | |
| Sucrose | 4.00 | 4.00 | 4.00 | 4.00 | 4.00 | 4.00 | 4.00 |
| Garlic | 0.50 | 0.50 | 0.50 | 0.50 | 0.50 | 0.50 | 0.50 |
| powder | | | | | | | |
| Hardened | 8.00 | 8.00 | 8.00 | 8.00 | 8.00 | 8.00 | 8.00 |
| vegetable | | | | | | | |
| fat | | | | | | | |
| Fat powder | 11.00 | 11.00 | 11.00 | 11.00 | 11.00 | 11.00 | 11.00 |
| Sodium | 6.00 | 6.00 | 6.00 | 6.00 | 6.00 | 6.00 | 6.00 |
| glutamate | | | | | | | |
| Food | 2.00 | 2.00 | 2.00 | 2.00 | 2.00 | 2.00 | 2.00 |
| coloring | | | | | | | |
| beet and | | | | | | | |
| paprika | | | | | | | |
| Aroma | 2.00 | 2.00 | 2.00 | 2.00 | 2.00 | 2.00 | 2.00 |
| Pepper″ | | | | | | | |
| type | | | | | | | |
| Aroma | 1.20 | 1.20 | 1.20 | 1.20 | 1.20 | 1.20 | 1.20 |
| Pizza″ type | | | | | | | |
| Aroma | 0.40 | 0.40 | 0.40 | 0.40 | 0.40 | 0.40 | 0.40 |
| Tomato″ | | | | | | | |
| type | | | | | | | |
| Black | 0.10 | 0.10 | 0.10 | 0.10 | 0.10 | 0.10 | 0.10 |
| pepper | | | | | | | |
| extract | | | | | | | |
| Aroma | — | 1.00 | — | — | — | — | — |
| composition | | | | | | | |
| (application | | | | | | | |
| example | | | | | | | |
| 1.1A) | | | | | | | |
| Aroma | — | — | 1.00 | — | — | — | — |
| composition | | | | | | | |
| (application | | | | | | | |
| example | | | | | | | |
| 1.1B) | | | | | | | |
| Aroma | — | — | — | 1.00 | — | — | — |
| composition | | | | | | | |
| (application | | | | | | | |
| example | | | | | | | |
| 1.1C) | | | | | | | |
| Aroma | — | — | — | — | 1.00 | — | — |
| composition | | | | | | | |
| (application | | | | | | | |
| example | | | | | | | |
| 1.1D) | | | | | | | |
| Aroma | — | — | — | — | — | 1.00 | — |
| composition | | | | | | | |
| (application | | | | | | | |
| example | | | | | | | |
| 1.1E) | | | | | | | |
| Aroma | — | — | — | — | — | — | 1.00 |
| composition | | | | | | | |
| (application | | | | | | | |
| example | | | | | | | |
| 1.1G) | | | | | | | |
| Total | 100 | 100 | 100 | 100 | 100 | 100 | 100 |

Note:
*not according to the invention

### Example 1.15: Seasoning Mix for Potato Chips

A=comparative preparation
B, C, D, E, F, G=preparations according to the invention

TABLE 29

| Component | A* | B | C | D | E | F | G |
|---|---|---|---|---|---|---|---|
| Sodium glutamate | 3.50 | 3.50 | 3.50 | 3.50 | 3.50 | 3.50 | 3.50 |
| Cheese powder | 10.00 | 10.00 | 10.00 | 10.00 | 10.00 | 10.00 | 10.00 |
| Garlic powder | 2.00 | 2.00 | 2.00 | 2.00 | 2.00 | 2.00 | 2.00 |
| Whey powder | 38.86 | 36.86 | 36.86 | 36.86 | 36.86 | 36.86 | 36.86 |
| Wort extract oil | 0.20 | 0.20 | 0.20 | 0.20 | 0.20 | 0.20 | 0.20 |
| Paprika powder | 9.80 | 9.80 | 9.80 | 9.80 | 9.80 | 9.80 | 9.80 |
| Common salt | 21.00 | 21.00 | 21.00 | 21.00 | 21.00 | 21.00 | 21.00 |
| Tomato powder | 9.00 | 9.00 | 9.00 | 9.00 | 9.00 | 9.00 | 9.00 |
| Dry flavor | 2.50 | 2.50 | 2.50 | 2.50 | 2.50 | 2.50 | 2.50 |
| Silicon dioxide | 0.02 | 0.02 | 0.02 | 0.02 | 0.02 | 0.02 | 0.02 |
| Vegetable oil | 0.02 | 0.02 | 0.02 | 0.02 | 0.02 | 0.02 | 0.02 |
| Onion powder | 3.00 | 3.00 | 3.00 | 3.00 | 3.00 | 3.00 | 3.00 |
| Cream flavor concentrate | 0.03 | 0.03 | 0.03 | 0.03 | 0.03 | 0.03 | 0.03 |
| Cheese flavor | 0.03 | 0.03 | 0.03 | 0.03 | 0.03 | 0.03 | 0.03 |
| Tomato flavor concentrate | 0.04 | 0.04 | 0.04 | 0.04 | 0.04 | 0.04 | 0.04 |
| Spray-dried composition | — | 2.00 | — | — | — | | |
| according to application example | | | | | | | |
| 1.2A | | | | | | | |
| Spray-dried composition | — | — | 2.00 | — | — | | — |
| according to application example | | | | | | | |
| 1.2B | | | | | | | |
| Spray-dried composition | — | — | — | 2.00 | — | | — |
| according to application example | | | | | | | |
| 1.2C | | | | | | | |
| Spray-dried composition | — | — | — | — | 2.00 | | — |
| according to application example | | | | | | | |
| 1.2D | | | | | | | |
| Spray-dried composition | | | | | | 2.00 | |
| according to application example | | | | | | | |
| 1.2E | | | | | | | |
| Spray-dried composition | — | — | — | — | — | | 2.00 |
| according to application example | | | | | | | |
| 1.2G | | | | | | | |
| Total | 100 | 100 | 100 | 100 | 100 | 100 | 100 |

Note:
*not according to the invention

6 g of the seasoning mix were added to 94 g of potato chips.

### Application Example 1.16: Use in a Green Tea Drink

TABLE 30

| | Weight % | Weight % |
|---|---|---|
| Component | A | B |
| Green tea concentrate | 18.00 | 18.00 |
| 1% solution | 0.02 | — |
| (3,4-Diethoxyphenyl)-[4-(2-phenoxy- | | |
| ethyl)-piperazin-1-yl]methanone | | |
| (8/13) | | |
| 1% solution | — | 0.015 |
| [4-(2-cyclohexylethyl)-piperazin-1- | | |
| yl]-phenyl-methanone (9/19) | | |
| Demineralized water | 81.98 | 81.985 |
| Total | 100 | 100 |

The green tea concentrate is mixed with the 1% solution of (3,4-diethoxyphenyl)-[4-(2-phenoxyethyl)-piperazin-1-yl]methanone (8/13) in propylene glycol in the case of beverage A and with the 1% solution of [4-(2-cyclohexylethyl)piperazin-1-yl]-phenyl-methanone (9/19) in propylene glycol in the case of beverage B. The green tea concentrate is mixed with the 1% solution of (3,4-diethoxyphenyl)[4-(2-phenoxyethyl)-piperazin-1-yl]methanone (8/13) in propylene glycol. Then make up to volume with demineralized water and mix thoroughly again. The product is then filtered, packaged ready for use and sterilized at 118° C. The taste of beverages A and B is rated by a panel of trained test subjects as clearly preferable to the unflavored green tea concentrate. The bitterness and astringency are reduced by the addition of the compounds according to the invention.

### Example 1.17: Use in an Iced Tea Drink (Black Tea)

The active ingredients according to the invention were each pre-dissolved 10% or 1% in ethanol. Black tea extract was dissolved in water and stirred together with sugar, a flavoring preparation (peach flavor) and the ethanolic solutions of the amides according to the invention in a beaker.

TABLE 31

| | Weight % | | | |
|---|---|---|---|---|
| Component | A | B | C | D |
| Black tea extract | 1.40 | 1.40 | 1.40 | 1.40 |
| Water | 89.51 | 89.5 | 89.49 | 89.515 |
| Flavor preparation (peach type) | 0.67 | 0.67 | 0.67 | 0.67 |
| Sugar | 7 | 7 | 7 | 7 |
| Citric acid (crystalline) | 1.20 | 1.20 | 1.20 | 1.20 |
| Ascorbic acid | 0.20 | 0.20 | 0.20 | 0.20 |
| 1% in ethanol | 0.02 | — | — | — |
| (3,4-Diethoxyphenyl)-[4-(2- | | | | |
| phenoxyethyl)piperazin-1- | | | | |
| yl]methanone (8/13) | | | | |
| 1% in ethanol | — | 0.03 | — | — |
| [4-(2-cyclohexylethyl)piperazin- | | | | |
| 1-yl]-phenyl-methanone (9/19) | | | | |
| 1% in ethanol | — | — | 0.04 | — |
| Phenyl-[4-(2-phenylethyl)-1,4- | | | | |
| diazepan-1-yl]methanon (44/22) | | | | |
| 1% in ethanol | — | — | — | 0.015 |
| (4-Methoxyphenyl)-[4- | | | | |
| (4-phenylbutyl)piperazin-1- | | | | |
| yl]methanone (13/34) | | | | |
| Total | 100 | 100 | 100 | 100 |

### Application Example 1.18: Application in a Bouillon

A=comparative preparation
B, C, D, E=preparations according to the invention

TABLE 32

| Component | A* | B | C | D | E |
|---|---|---|---|---|---|
| Fat powder | 8.77 | 8.77 | 8.77 | 8.77 | 8.77 |
| Sodium glutamate | 8.77 | 8.77 | 8.77 | 8.77 | 8.77 |
| Yeast extract powder | 12.28 | 12.28 | 12.28 | 12.28 | 12.28 |
| Common salt | 29.83 | 29.83 | 29.83 | 29.83 | 29.83 |
| Maltodextrin | 37.28 | 37.23 | 37.18 | 37.13 | 37.265 |
| Natural vegetable | 3.07 | 3.07 | 3.07 | 3.07 | 3.07 |
| extract | | | | | |
| (3,4-Diethoxyphenyl)- | — | 0.05 | — | — | — |
| [4-(2-phenoxyethyl)- | | | | | |
| piperazin-1- | | | | | |
| yl]methanone (8/13) | | | | | |
| [4-(2- | — | — | 0.1 | — | — |
| cyclohexylethyl)- | | | | | |
| piperazin-1-yl]- | | | | | |
| phenyl-methanone | | | | | |
| (9/19) | | | | | |
| Phenyl-[4-(2-phenyl- | — | — | — | 0.15 | — |
| ethyl)-1,4-diazepan- | | | | | |
| 1-yl]methanon (44/22) | | | | | |
| (4-Methoxyphenyl)- | — | — | — | — | 0.015 |
| [4-(4- | | | | | |
| phenylbutyl)piperazin- | | | | | |
| 1-yl]methanone (13/34) | | | | | |
| Total | 100 | 100 | 100 | 100 | 100 |

Note:
*not according to the invention

15 g of each powder mixture was infused with 1000 mL of hot water.

### Example 1.19: Instant Soup, Leek Cream Type

A=comparative preparation
B, C, D, E, F=preparations according to the invention

TABLE 33

| Component | A* | B | C | D | E | F |
|---|---|---|---|---|---|---|
| Potato starch | 20.00 | 20.00 | 20.00 | 20.00 | 20.00 | 20.00 |
| Fat powder | 25.00 | 25.00 | 25.00 | 25.00 | 25.00 | 25.00 |
| Lactose | 20.00 | 20.00 | 20.00 | 20.00 | 20.00 | 20.00 |
| Maltodextrin | 11.73 | 11.72 | 11.725 | 11.67 | 11.722 | 11.727 |
| Common salt | 8.00 | 8.00 | 8.00 | 8.00 | 8.00 | 8.00 |
| Sodium | 3.00 | 3.00 | 3.00 | 3.00 | 3.00 | 3.00 |
| glutamate | | | | | | |
| Spinach powder | 2.00 | 2.00 | 2.00 | 2.00 | 2.00 | 2.00 |
| Green leek | 2.00 | 2.00 | 2.00 | 2.00 | 2.00 | 2.00 |
| powder | | | | | | |
| Citric acid, as | 0.30 | 0.30 | 0.30 | 0.30 | 0.30 | 0.30 |
| powder | | | | | | |
| Hardened | 3.00 | 3.00 | 3.00 | 3.00 | 3.00 | 3.00 |
| vegetable | | | | | | |
| fat | | | | | | |
| Freeze-dried leek | 1.00 | 1.00 | 1.00 | 1.00 | 1.00 | 1.00 |
| Chicken flavor | 1.00 | 1.00 | 1.00 | 1.00 | 1.00 | 1.00 |
| Seasoning | 2.00 | 2.00 | 2.00 | 2.00 | 2.00 | 2.00 |
| mix type | | | | | | |
| “green leek”, | | | | | | |
| powder | | | | | | |
| Seasoning mix, | 0.60 | 0.60 | 0.60 | 0.60 | 0.60 | 0.60 |
| “cooked | | | | | | |
| onion” type | | | | | | |
| Yeast seasoning | 0.30 | 0.30 | 0.30 | 0.30 | 0.30 | 0.30 |
| mix, “vegetable | | | | | | |
| stock” type, | | | | | | |
| powder | | | | | | |
| Curcuma extract | 0.07 | 0.07 | 0.07 | 0.07 | 0.07 | 0.07 |
| (3,4- | — | 0.01 | — | — | 0.006 | |
| Diethoxy- | | | | | | |
| phenyl)-[4-(2- | | | | | | |
| phenoxyethyl)- | | | | | | |
| piperazin-1- | | | | | | |
| yl]methanone | | | | | | |
| (8/13) | | | | | | |
| [4-(2- | — | — | 0.005 | — | — | |
| cyclohexylethyl)- | | | | | | |
| piperazin-1-yl]- | | | | | | |
| phenyl- | | | | | | |
| methanone | | | | | | |
| (9/19) | | | | | | |
| Phenyl-[4-(2- | — | — | — | 0.06 | 0.002 | |
| phenylethyl)-1,4- | | | | | | |
| diazepan-1- | | | | | | |
| yl]methanon | | | | | | |
| (44/22) | | | | | | |
| (4-Methoxy- | | | | | | 0.003 |
| phenyl)- | | | | | | |
| [4-(4- | | | | | | |
| phenylbutyl)- | | | | | | |
| piperazin-1- | | | | | | |
| yl]methanone | | | | | | |
| (13/34) | | | | | | |
| Total | 100 | 100 | 100 | 100 | 100 | 100 |

Note:
*not according to the invention

5 g of each powder mixture was infused with 100 ml of hot water to obtain a ready-to-eat soup.

### Example 1.20: Instant Soup, Type Chicken Soup with Noodles

A=comparative preparation
B, C, D, E, F=preparations according to the invention

TABLE 34

| Component | A* | B | C | D | E | F |
|---|---|---|---|---|---|---|
| Strength | 16.16 | 16.15 | 16.12 | 16.13 | 16.11 | 16.155 |
| Common salt | 7.00 | 7.00 | 7.00 | 7.00 | 7.00 | 7.00 |
| Sucrose, refined | 3.20 | 3.20 | 3.20 | 3.20 | 3.20 | 3.20 |
| Sodium | 3.20 | 3.20 | 3.20 | 3.20 | 3.20 | 3.20 |
| glutamate | | | | | | |
| Sodium | 0.80 | 0.80 | 0.80 | 0.80 | 0.80 | 0.80 |
| inosinate/ | | | | | | |
| Sodium | | | | | | |
| guanylate in a | | | | | | |
| ratio of 1:1 | | | | | | |
| Acid hydrolyzed | 8.00 | 8.00 | 8.00 | 8.00 | 8.00 | 8.00 |
| plant | | | | | | |
| protein | | | | | | |
| Fat powder | 2.00 | 2.00 | 2.00 | 2.00 | 2.00 | 2.00 |
| Vegetable fat, | 1.00 | 1.00 | 1.00 | 1.00 | 1.00 | 1.00 |
| spray-dried | | | | | | |
| Freeze-dried | 2.00 | 2.00 | 2.00 | 2.00 | 2.00 | 2.00 |
| chicken, in | | | | | | |
| pieces | | | | | | |
| Soup noodles | 32.00 | 32.00 | 32.00 | 32.00 | 32.00 | 32.00 |
| Maltodextrin | 12.00 | 12.00 | 12.00 | 12.00 | 12.00 | 12.00 |
| Chinese | 4.60 | 4.60 | 4.60 | 4.60 | 4.60 | 4.60 |
| vegetables, | | | | | | |
| freeze-dried | | | | | | |
| Chicken flavor | 8.00 | 8.00 | 8.00 | 8.00 | 8.00 | 8.00 |
| Food coloring | 0.04 | 0.04 | 0.04 | 0.04 | 0.04 | 0.04 |
| riboflavin | | | | | | |
| (3,4-Diethoxy- | — | 0.01 | — | — | 0.04 | — |
| phenyl)-[4- | | | | | | |
| (2- | | | | | | |
| phenoxy- | | | | | | |
| ethyl)piperazin- | | | | | | |
| 1-yl]methanone | | | | | | |
| (8/13) | | | | | | |
| [4-(2- | — | — | 0.04 | — | 0.01 | — |
| cyclohexylethyl)- | | | | | | |
| piperazin- | | | | | | |
| 1-yl]-phenyl- | | | | | | |
| methanone (9/19) | | | | | | |
| Phenyl-[4-(2- | — | — | — | 0.03 | — | — |
| phenylethyl)-1,4- | | | | | | |
| diazepan-1- | | | | | | |
| yl]methanon | | | | | | |
| (44/22) | | | | | | |
| (4-Methoxy- | — | — | — | — | — | 0.005 |
| phenyl)-[4-(4- | | | | | | |
| phenyl-butyl) | | | | | | |
| piperazin-1- | | | | | | |
| yl]methanone | | | | | | |
| (13/34) | | | | | | |
| Total | 100 | 100 | 100 | 100 | 100 | 100 |

Note:
*not according to the invention

4.60 g of each powder mixture was boiled in 100 ml of water for 10 minutes to obtain a ready-to-eat soup.

### Example 1.21: Production of Dark Chili Chocolates Using the Substances

### According to the Invention

A=Comparison preparation dark chocolate
B=low-calorie dark chocolate
C=calorie-reduced dark chocolate
D=low-calorie dark chocolate
E=reduced-calorie milk chocolate
F=dark chocolate
G=dark chocolate

TABLE 35

| Component | A* | B | C | D | E | F | G |
|---|---|---|---|---|---|---|---|
| Cocoa butter | 14.49 | 12.99 | 13.41 | 9.48 | 14.00 | 14.48 | 14.498 |
| Cocoa paste | 41.00 | 39.00 | 42.00 | 44.00 | 23.00 | 41.00 | 41.00 |
| Erythritol | — | 47.45 | — | — | — | — | — |
| Maltitol, crystalline | — | — | — | 23.00 | | — | — |
| Inulin | — | — | — | 23.00 | | — | — |
| Sorbitol | — | — | 44.00 | — | — | — | — |
| Lactitol | — | — | — | — | 38.55 | — | — |
| Polydextrose | — | — | — | — | 9.70 | — | — |
| Whole milk powder | — | — | — | — | 14.00 | — | — |
| Sucrose | 43.98 | — | — | — | — | 44.00 | 44.00 |
| Lecitin | 0.48 | 0.48 | 0.40 | 0.48 | 0.50 | 0.48 | 0.48 |
| Vanillin | 0.02 | 0.02 | 0.02 | 0.02 | 0.20 | 0.02 | 0.02 |
| Aspartame | — | 0.03 | 0.06 | — | 0.03 | — | — |
| Capsicum extract | 0.03 | 0.02 | 0.02 | 0.01 | — | 0.01 | — |
| (1,000,000 SHU) | | | | | | | |
| (3,4-Diethoxyphenyl)-[4- | — | 0.01 | — | 0.01 | 0.01 | — | — |
| (2- | | | | | | | |
| phenoxyethyl)piperazin- | | | | | | | |
| [symbol] | | | | | | | |
| [4-(2-cyclohexylethyl)- | — | — | 0.01 | — | 0.01 | — | — |
| piperazin-1-yl]-phenyl- | | | | | | | |
| methanone (9/19) | | | | | | | |
| Phenyl-[4-(2- | — | — | — | — | — | 0.01 | — |
| phenylethyl)-1,4- | | | | | | | |
| diazepan-1-yl]methanon | | | | | | | |
| (4-Methoxyphenyl)-[4- | — | — | — | — | — | — | 0.002 |
| (4- | | | | | | | |
| phenylbutyl)piperazin-1- | | | | | | | |
| Total | 100 | 100 | 100 | 100 | 100 | 100 | 100 |

Note:
*not according to the invention [symbol]
indicates data missing or illegible when filed

The effects found in the above examples of use can be transferred to all products of the respective product group, i.e., in particular to toothpastes, chewing gums, mouthwashes, throat lozenges, gelatine capsules, chewy sweets and tea in sachets, if necessary by means of modifications that are not difficult for the skilled person to make. It is not difficult for a person skilled in the art to recognize from the present description that the compounds and mixtures according to the invention are interchangeable with one another without great effort—possibly with minor modifications. This means that the compound according to the invention used in the products of the application examples must also be understood as a placeholder for the other compounds and mixtures according to the invention.

The concentration of the compound or mixture used according to the invention is also easily recognizable to the skilled person. In addition, the product-specific further components in the respective application example can also be easily replaced or supplemented by further product-typical components in a manner that is easily comprehensible to the skilled person. A large number of such product-specific components are disclosed in the above description.

1. Active ingredient of the general formula (I) 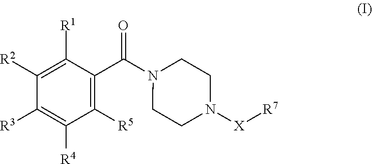
or the general formula (II) 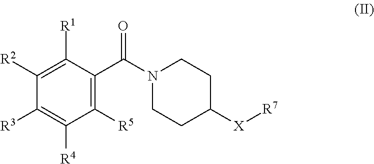
or the general formula (III) 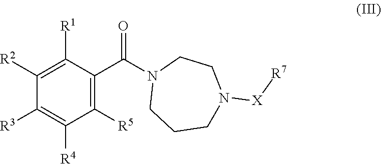
wherein the radicals R1 to R5 and R7 can each be identical or different and independently of one another have the following meanings:
R1
H; or
OH; or
Q; or
an optionally substituted alkyl group; or
an optionally substituted alkenyl group; or
an optionally substituted alkoxy group; or
an optionally substituted alkylthio group; or
an optionally substituted acyl group R—(C═O)—; or
an optionally substituted cycloalkyl group; or
an optionally substituted aryl group; or
an optionally substituted heterocyclyl group; or
is an optionally substituted heteroaryl group; or
R1 forms with the adjacent R2, together with the carbon atoms to which they are attached, an optionally substituted four-, five-, six- or seven-membered cycloalkyl or aryl ring, or forms an optionally substituted four-, five-, six- or seven-membered heterocyclyl or heteroaryl ring, wherein the heterocyclyl or heteroaryl ring may comprise one, two or more identical or different heteroatoms selected from the group consisting of nitrogen, oxygen and sulfur;
R2
H; or
OH; or
Q; or
an optionally substituted alkyl group; or
an optionally substituted alkenyl group; or
an optionally substituted alkoxy group; or
an optionally substituted alkylthio group; or
an optionally substituted acyl group R—(C═O)—; or
an optionally substituted cycloalkyl group; or
an optionally substituted aryl group; or
an optionally substituted heterocyclyl group; or
is an optionally substituted heteroaryl group; or
R2 forms with one of the adjacent radicals R1 or R3, together with the carbon atoms to which they are attached, an optionally substituted four-, five-, six- or seven-membered cycloalkyl or aryl ring, or forms an optionally substituted four-, five-, six- or seven-membered heterocyclyl or heteroaryl ring, wherein the heterocyclyl or heteroaryl ring may comprise one, two or more identical or different heteroatoms selected from the group consisting of nitrogen, oxygen and sulfur; and/or
R3
H; or
OH; or
Q; or
an optionally substituted alkyl group; or
an optionally substituted alkenyl group; or
an optionally substituted alkoxy group; or
an optionally substituted alkylthio group; or
an optionally substituted acyl group R—(C═O)—; or
an optionally substituted cycloalkyl group; or
an optionally substituted aryl group; or
an optionally substituted heterocyclyl group; or
is an optionally substituted heteroaryl group; or
R3 forms with one of the adjacent R2 or R4, together with the carbon atoms to which they are attached, an optionally substituted four-, five-, six- or seven-membered cycloalkyl or aryl ring, or forms an optionally substituted four-, five-, six- or seven-membered heterocyclyl or heteroaryl ring, wherein the heterocyclyl or heteroaryl ring may comprise one, two or more identical or different heteroatoms selected from the group consisting of nitrogen, oxygen and sulfur;
R4
H; or
OH; or
Q; or
an optionally substituted alkyl group; or
an optionally substituted alkenyl group; or
an optionally substituted alkoxy group; or
an optionally substituted alkylthio group; or
an optionally substituted acyl group R—(C═O)—; or
an optionally substituted cycloalkyl group; or
an optionally substituted aryl group; or
an optionally substituted heterocyclyl group; or
is an optionally substituted heteroaryl group; or
R4 forms with one of the adjacent R3 or R5, together with the carbon atoms to which they are attached, an optionally substituted four-, five-, six- or seven-membered cycloalkyl or aryl ring, or forms an optionally substituted four-, five-, six- or seven-membered heterocyclyl or heteroaryl ring, wherein the heterocyclyl or heteroaryl ring may comprise one, two or more identical or different heteroatoms selected from the group consisting of nitrogen, oxygen and sulfur; and/or
R5
H; or
OH; or
Q; or
an optionally substituted alkyl group; or
an optionally substituted alkenyl group; or
an optionally substituted alkoxy group; or
an optionally substituted alkylthio group; or
an optionally substituted acyl group R—(C═O)—; or
an optionally substituted cycloalkyl group; or
an optionally substituted aryl group; or
an optionally substituted heterocyclyl group; or
is an optionally substituted heteroaryl group; or
R5 forms with the adjacent radical R4, together with the carbon atoms to which they are attached, an optionally substituted four-, five-, six- or seven-membered cycloalkyl or aryl ring or forms an optionally substituted four-, five-, six- or seven-membered heterocyclyl or heteroaryl ring, wherein the heterocyclyl or heteroaryl ring may comprise one, two or more identical or different heteroatoms selected from the group consisting of nitrogen, oxygen and sulfur; and/or
R7
an optionally substituted cycloalkyl group; or
an optionally substituted aryl group; or
an optionally substituted heterocyclyl group; or
is an optionally substituted heteroaryl group;
X
an optionally substituted alkyl group; or
an optionally substituted alkenyl group; or
an optionally substituted alkoxy group; or
an optionally substituted alkylthio group; or
is an optionally substituted acyl group R(C═O)—;
optionally one or both nitrogen atom(s) of the saturated six-membered ring in the general formula (I) or the nitrogen atom of the saturated six-membered ring in the general formula (II) or one or both nitrogen atom(s) of the saturated seven-membered ring in the general formula (III) is/are replaced by a C atom;
optionally the saturated six-membered ring in the general formula (I) or (II) or the saturated seven-membered ring in the general formula (III) is mono- or polysubstituted;
wherein
the group Q is a radical selected from the group consisting of: halogen, —OY, —SY, —NZZ, —NY, —N—OY, trihalomethyl, —CF₃, —CN, —OCN, —SCN, —NO, —NO₂, —NH₂, —N₃, —S(O)₂Y, —S(O)₂OY, —OS(O)₂Y, —OS(O)₂OY, —P(O)(OY)₂, —P(O)(OY)(OY), —C(O)Y, —C(S)Y, —C(NY)Y, —C(O)OY, —C(S)OY, —C(O)NZZ, —C(NY)NZZ, —OC(O)Y, —OC(S)Y, —OC(O)OY, —OC(S)OY, —NYC(O)Y, —NYC(S)Y, —NYC(O)OY, —NYC(S)OY, —NYC(O)NZZ, —NYC(NY)Y or —NYC(NY)NZZ;
wherein
Y is selected from the group consisting of:
Hydrogen;
alkyl group;
alkoxy group;
alkylthio group;
Cycloalkyl group;
Aryl Group;
Heterocyclyl group;
heteroaryl group; and/or
Z represents Y or alternatively, two Z's together with the nitrogen atom to which they are attached represent a four, five, six or seven membered heterocyclyl ring or heteroaryl ring, wherein the heterocyclyl ring or heteroaryl ring may comprise one, two or more identical or different heteroatoms selected from the group consisting of nitrogen, oxygen and sulfur;
the optionally substituted alkyl group is an optionally substituted C₁- to C₁₀-alkyl group;
the optionally substituted alkenyl group is an optionally substituted C₁- to C₁₀-alkenyl group;
the optionally substituted alkoxy group is an optionally substituted C₁- to C₁₀-alkoxy group;
the optionally substituted alkylthio group is an optionally substituted C₁- to C₁₀-alkylthio group;
in the acyl group R—(C═O)— the radical R represents hydrogen or an optionally substituted C₁- to C₁₀-alkyl group;
the optionally substituted cycloalkyl group is an optionally substituted four- to ten-membered, monocyclic cycloalkyl group or an optionally substituted nine- to twelve-membered polycyclic cycloalkyl group;
the optionally substituted aryl group is an optionally substituted four- to ten-membered monocyclic aryl group or an optionally substituted nine- to twelve-membered polycyclic aryl group;
the optionally substituted heterocyclyl group is an optionally substituted four-, five-, six- or seven-membered monocyclic heterocyclyl group or an optionally substituted nine- to twelve-membered polycyclic, heterocyclyl group, wherein the heterocyclyl group may comprise one, two, three or four identical or different heteroatoms selected from the group consisting of nitrogen, oxygen and sulfur;
the optionally substituted heteroaryl group is an optionally substituted four-, five-, six- or seven-membered monocyclic heteroaryl group or an optionally substituted nine- to twelve-membered polycyclic, heteroaryl group, wherein the heteroaryl group may comprise one, two, three or four identical or different heteroatoms selected from the group consisting of nitrogen, oxygen and sulfur;
or salts thereof, wherein the active ingredients are present in stereoisomerically pure form or a mixture of different stereoisomers.

2. The active ingredient according to claim 1, wherein in the general formula (I)
R1, R4 and R5 each represent H independently of each other;
R2 and/or R3 are identical or different and each independently represents H, OH or an optionally substituted C₁-, C₂-, C₃- or C₄-alkoxy group;
X stands for an optionally substituted C₁-, C₂-, C₃- or C₄-alkyl group or an optionally substituted C₁-, C₂-, C₃- or C₄-alkenyl group or an optionally substituted C₁-, C₂-, C₃- or C₄-alkoxy group;
R7 represents an optionally substituted five or six membered cycloalkyl group or an optionally substituted five or six membered aryl group or an optionally substituted five or six membered heterocyclyl group having one, two or more identical or different heteroatom(s) or an optionally substituted five or six membered heteroaryl group having one, two or more identical or different heteroatom(s), wherein the heteroatoms are selected from the group consisting of nitrogen, oxygen and sulfur;
optionally one or both nitrogen atom(s) of the saturated six-membered ring in the general formula (I) is/are replaced by a C atom;
optionally the saturated six-membered ring in the general formula (I) is mono- or polysubstituted; or
wherein in the general formula (II)
R1, R4 and R5 each represent H independently of each other;
R2 and/or R3 are identical or different and each independently represents H, OH or an optionally substituted C₁-, C₂-, C₃- or C₄-alkoxy group;
X stands for an optionally substituted C₁-, C₂-, C₃- or C₄-alkyl group or an optionally substituted C₁-, C₂-, C₃- or C₄-alkenyl group or an optionally substituted C₁-, C₂-, C₃- or C₄-alkoxy group;
R7 represents an optionally substituted five or six membered cycloalkyl group or an optionally substituted five or six membered aryl group or an optionally substituted five or six membered heterocyclyl group having one, two or more identical or different heteroatom(s) or an optionally substituted five or six membered heteroaryl group having one, two or more identical or different heteroatom(s), wherein the heteroatoms are selected from the group consisting of nitrogen, oxygen and sulfur;
optionally the nitrogen atom of the saturated six-membered ring in the general formula (II) is replaced by a C atom;
optionally the saturated six-membered ring in the general formula (II) is mono- or polysubstituted; or
wherein in the general formula (III)
R1, R4 and R5 each represent H independently of each other;
R2 and/or R3 are identical or different and each independently represents H, OH or an optionally substituted C₁-, C₂-, C₃- or C₄-alkoxy group;
X stands for an optionally substituted C₁-, C₂-, C₃- or C₄-alkyl group or an optionally substituted C₁-, C₂-, C₃- or C₄-alkenyl group or an optionally substituted C₁-, C₂-, C₃- or C₄-alkoxy group;
R7 represents an optionally substituted five or six membered cycloalkyl group or an optionally substituted five or six membered aryl group or an optionally substituted five or six membered heterocyclyl group having one, two or more identical or different heteroatom(s) or an optionally substituted five or six membered heteroaryl group having one, two or more identical or different heteroatom(s), wherein the heteroatoms are selected from the group consisting of nitrogen, oxygen and sulfur;
optionally one or both nitrogen atom(s) of the saturated seven-membered ring in the general formula (III) is/are replaced by a C atom;
optionally the saturated seven-membered ring in the general formula (III) is mono- or polysubstituted.

3. The active ingredient according to claim 1, wherein in the general formula (I)
R1, R4 and R5 each represent H independently of each other;
R2 and/or R3 are identical or different and each independently represents H, OH or an optionally substituted C₁—, C₂- or C₃-alkoxy group;
X stands for an optionally substituted C₁-, C₂-, C₃- or C₄-alkyl group or an optionally substituted C₁-, C₂-, C₃- or C₄-alkenyl group or an optionally substituted C₁-, C₂-, C₃- or C₄-alkoxy group;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally one or both nitrogen atom(s) of the saturated six-membered ring in the general formula (I) is/are replaced by a C atom;
optionally the saturated six-membered ring in the general formula (I) is mono- or polysubstituted; or
wherein in the general formula (II)
R1, R4 and R5 each represent H independently of each other;
R2 and/or R3 are identical or different and each independently represents H, OH or an optionally substituted C₁—, C₂- or C₃-alkoxy group;
X stands for an optionally substituted C₁-, C₂-, C₃- or C₄-alkyl group or an optionally substituted C₁-, C₂-, C₃- or C₄-alkenyl group or an optionally substituted C₁-, C₂-, C₃- or C₄-alkoxy group;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally the nitrogen atom of the saturated six-membered ring in the general formula (II) is replaced by a C atom;
optionally the saturated six-membered ring in the general formula (II) is mono- or polysubstituted; or
wherein in the general formula (III)
R1, R4 and R5 each represent H independently of each other;
R2 and/or R3 are identical or different and each independently represents OH or an optionally substituted C₁—, C₂- or C₃-alkoxy group;
X stands for an optionally substituted C₁-, C₂-, C₃- or C₄-alkyl group or an optionally substituted C₁-, C₂-, C₃- or C₄-alkenyl group or an optionally substituted C₁-, C₂-, C₃- or C₄-alkoxy group;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally one or both nitrogen atom(s) of the saturated seven-membered ring in the general formula (III) is/are replaced by a C atom;
optionally the saturated seven-membered ring in the general formula (III) is mono- or polysubstituted.

4. The active ingredient according to claim 1, wherein in the general formula (I) or the general formula (II) or the general formula (III)
R1, R4 and R5 each represent H independently of each other;
R2 and/or R3 are the same or different and each independently represents H or OH or C1 alkoxy or C2 alkoxy or C3 alkoxy and X represents C3 alkenyl, or
R2 and/or R3 are the same or different and each independently represents H or OH or C1 alkoxy or C2 alkoxy or C3 alkoxy and X represents C4 alkyl, or
R2 and/or R3 are the same or different and each independently represents H or OH or C1 alkoxy or C2 alkoxy or C3 alkoxy and X represents C3 alkyl, or
R2 and/or R3 are the same or different and each independently represents H or OH or C1-alkoxy or C2-alkoxy or C3-alkoxy and X represents C2-alkyl, or
R2 and/or R3 are the same or different and each independently represents H or OH or C1 alkoxy or C2 alkoxy or C3 alkoxy and X represents C1 alkyl, or
R2 and/or R3 are the same or different and each independently represents H or OH or C1 alkoxy or C2 alkoxy or C3 alkoxy and X represents C2 alkoxy;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally or one or both nitrogen atom(s) of the saturated six-membered ring in the general formula (I) or the nitrogen atom of the saturated six-membered ring in the general formula (II) or one or both nitrogen atom(s) of the seven-membered ring in the general formula (III) is/are replaced by a C atom;
optionally the saturated six-membered ring in the general formula (I) or in the general formula (II) or the saturated seven-membered ring in the general formula (III) is mono- or polysubstituted.

5. The active ingredient according to claim 1, wherein in the general formula (I)
R1, R4 and R5 each independently represent H; and
R2 and/or R3 are the same or different and each independently represents H, OH, C1-alkoxy, C2-alkoxy or C3-alkoxy and X represents C2-alkyl, C3-alkyl, C4-alkyl, C3-alkenyl or C2-alkoxy;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally one or both nitrogen atom(s) of the saturated six-membered ring in the general formula (I) is/are replaced by a C atom;
optionally the saturated six-membered ring in the general formula (I) is mono- or polysubstituted; or
wherein in the general formula (II)
R1, R4 and R5 each represent H independently of each other;
R2 and/or R3 are the same or different and each independently represents H, C1-alkoxy or C2-alkoxy and X represents C3-alkyl;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally the nitrogen atom of the saturated six-membered ring in the general formula (II) is replaced by a C atom;
optionally the saturated six-membered ring in the general formula (II) is mono- or polysubstituted; or
wherein in the general formula (III)
R1, R4 and R5 each represent H independently of each other;
R2 and/or R3 are the same or different and each independently represents H, C1 alkoxy or C2 alkoxy and X represents C2 alkyl;
R7 represents an optionally substituted six-membered cycloalkyl group or an optionally substituted six-membered aryl group or an optionally substituted six-membered heterocyclyl group having one, two or more identical or different heteroatom(s) or an optionally substituted six-membered heteroaryl group having one, two or more identical or different heteroatom(s), wherein the heteroatoms are selected from the group consisting of nitrogen, oxygen and sulfur;
optionally one or both nitrogen atom(s) of the saturated seven-membered ring in the general formula (III) is/are replaced by a C atom;
optionally the saturated seven-membered ring in the general formula (III) is mono- or polysubstituted.

6. The active ingredient according to claim 1, wherein the active ingredient of general formula (I) is selected from the group consisting of:

| Comp. | | | Chemical |
|---|---|---|---|
| No. | | Compound | Designation |
| 1 | 1 | 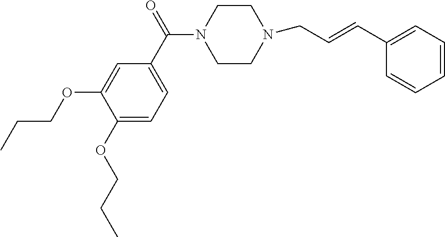 [SMILES: CCCOc1ccc(C(=O)N2CCN(C/C=C/c3ccccc3)CC2)cc1OCCC] | [4-[(E)-cinnamyl]piperazin- 1-yl]-(3,4- dipropoxyphenyl)methanone |
| 2 | 4 | 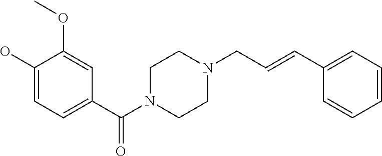 [SMILES: COc1cc(C(=O)N2CCN(C/C=C/c3ccccc3)CC2)ccc1O] | [4-[(E)-cinnamyl]piperazin- 1-yl]-(4-hydroxy-3- methoxy-phenyl)methanone |
| 3 | 6 | 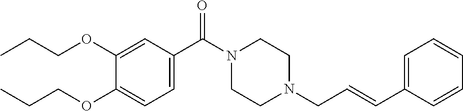 [SMILES: CCCOc1ccc(C(=O)N2CCN(C/C=C/c3ccccc3)CC2)cc1OCCC] | [4-[(E)-cinnamyl]piperazin- 1-yl]-(3,4- dipropoxyphenyl)methanone |
| 4 | 9 | 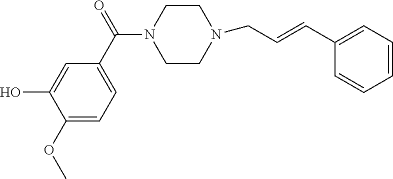 [SMILES: COc1ccc(C(=O)N2CCN(C/C=C/c3ccccc3)CC2)cc1O] | [4-[(E)-cinnamyl]piperazin- 1-yl]-(3-hydroxy-4- methoxy-phenyl)methanone |
| 5 | 10 | 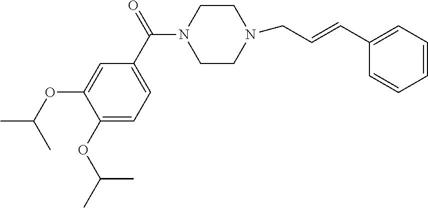 [SMILES: CC(C)Oc1ccc(C(=O)N2CCN(C/C=C/c3ccccc3)CC2)cc1OC(C)C] | [4-[(E)-cinnamyl]piperazin- 1-yl]-(3,4- diisopropoxyphenyl)methan one |
| 6 | 11 | 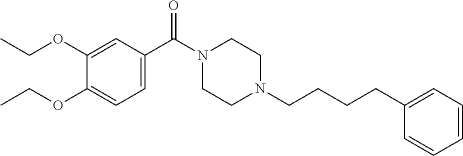 [SMILES: CCOc1ccc(C(=O)N2CCN(CCCCc3ccccc3)CC2)cc1OCC] | (3,4-Diethoxyphenyl)-[4-(4- phenylbutyl)piperazin-1- yl]methanone |
| 7 | 12 | 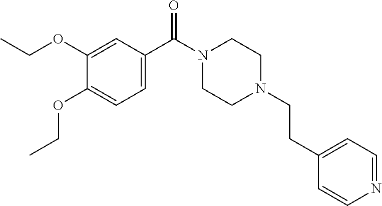 [SMILES: CCOc1ccc(C(=O)N2CCN(CCc3ccncc3)CC2)cc1OCC] | (3,4-Diethoxyphenyl)-[4-[2- (4-pyridyl)ethyl]piperazin-1- yl]methanone |
| 8 | 13 | 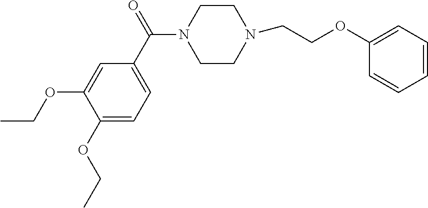 [SMILES: CCOc1ccc(C(=O)N2CCN(CCOc3ccccc3)CC2)cc1OCC] | (3,4-Diethoxyphenyl)-[4-(2- phenoxyethyl)piperazin-1- yl]methanone |
| 9 | 19 | 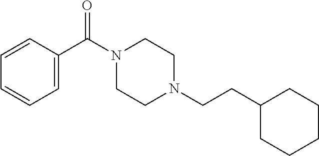 [SMILES: O=C(c1ccccc1)N1CCN(CCC2CCCCC2)CC1] | [4-(2- cyclohexylethyl)piperazin-1- yl]-phenyl-methanone |
| 10 | 24 | 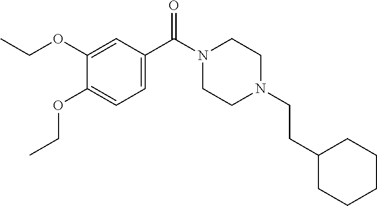 [SMILES: CCOc1ccc(C(=O)N2CCN(CCC3CCCCC3)CC2)cc1OCC] | [4-(2- cyclohexylethyl)piperazin-1- yl]-(3,4- diethoxyphenyl)methanone |
| 11 | 27 | 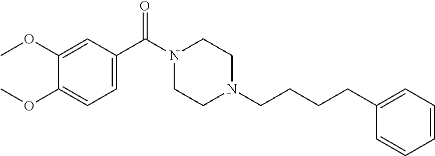 [SMILES: COc1ccc(C(=O)N2CCN(CCCCc3ccccc3)CC2)cc1OC] | (3,4-Dimethoxyphenyl)-[4- (4-phenylbutyl)piperazin-1- yl]methanone |
| 12 | 29 | 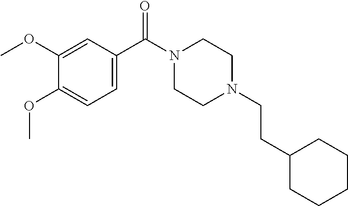 [SMILES: COc1ccc(C(=O)N2CCN(CCC3CCCCC3)CC2)cc1OC] | [4-(2- cyclohexylethyl)piperazin-1- yl]-(3,4- dimethoxyphenyl)methanone |
| 13 | 34 | 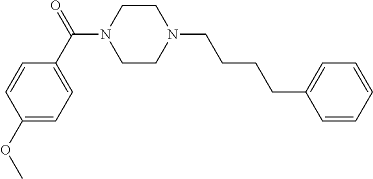 [SMILES: COc1ccc(C(=O)N2CCN(CCCCc3ccccc3)CC2)cc1] | (4-Methoxyphenyl)-[4-(4- phenylbutyl)piperazin-1- yl]methanone |
| 14 | 36 | 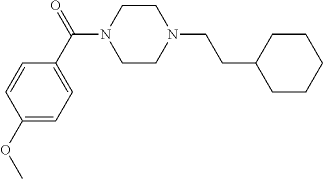 [SMILES: COc1ccc(C(=O)N2CCN(CCC3CCCCC3)CC2)cc1] | [4-(2- cyclohexylethyl)piperazin-1- yl]-(4- methoxyphenyl)methanone |
| 15 | 41 | 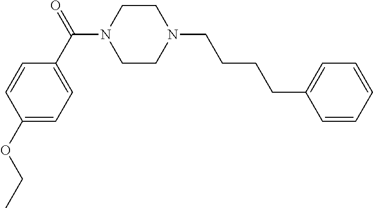 [SMILES: CCOc1ccc(C(=O)N2CCN(CCCCc3ccccc3)CC2)cc1] | (4-ethoxyphenyl)-[4-(4- phenylbutyl)piperazin-1- yl]methanone |
| 16 | 44 | 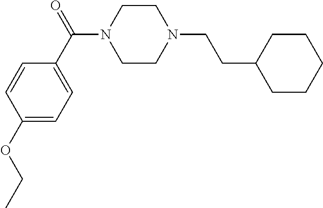 [SMILES: CCOc1ccc(C(=O)N2CCN(CCC3CCCCC3)CC2)cc1] | [4-(2- cyclohexylethyl)piperazin-1- yl]-(4- ethoxyphenyl)methanone |
| 17 | 2 | 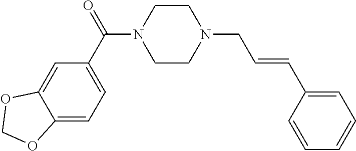 [SMILES: O=C(c1ccc2c(c1)OCO2)N1CCN(C/C=C/c2ccccc2)CC1] | 1,3-benzodioxol-5-yl-[4- [(E)-cinnamyl]piperazin-1- yl]methanone |
| 18 | 3 | 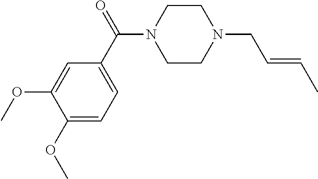 [SMILES: C/C=C/CN1CCN(C(=O)c2ccc(OC)c(OC)c2)CC1] | [4-[(E)-cinnamyl]piperazin- 1-yl]-(3,4- dimethoxyphenyl)methanone |
| 19 | 5 | 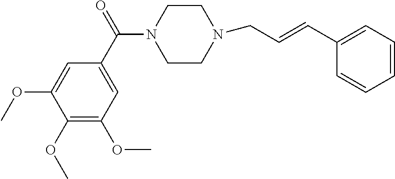 [SMILES: COc1cc(C(=O)N2CCN(C/C=C/c3ccccc3)CC2)cc(OC)c1OC] | [4-[(E)-cinnamyl]piperazin- 1-yl]-(3,4,5- trimethoxyphenyl)methanone |
| 20 | 7 | 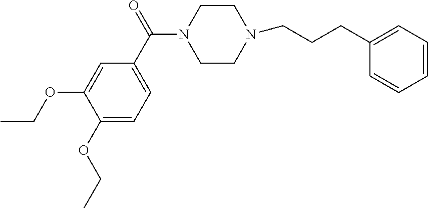 [SMILES: CCOc1ccc(C(=O)N2CCN(CCCc3ccccc3)CC2)cc1OCC] | (3,4-Diethoxyphenyl)-[4-(3- phenylpropyl)piperazin-1- yl]methanone |
| 21 | 8 | 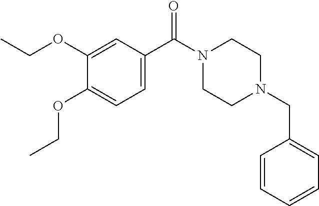 [SMILES: CCOc1ccc(C(=O)N2CCN(Cc3ccccc3)CC2)cc1OCC] | (4-benzylpiperazin-1-yl)- (3,4- diethoxyphenyl)methanone |
| 22 | 14 | 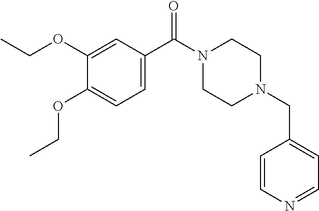 [SMILES: CCOc1ccc(C(=O)N2CCN(Cc3ccncc3)CC2)cc1OCC] | (3,4-Diethoxyphenyl)-[4-(4- pyridylmethyl)piperazin-1- yl]methanone |
| 23 | 15 | 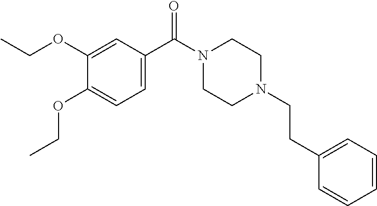 [SMILES: CCOc1ccc(C(=O)N2CCN(CCc3ccccc3)CC2)cc1OCC] | (3,4-Diethoxyphenyl)-[4-(2- phenylethyl)piperazin-1- yl]methanone |
| 24 | 16 | 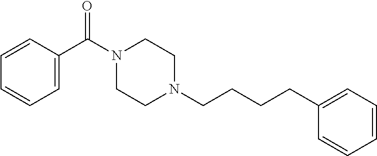 [SMILES: O=C(c1ccccc1)N1CCN(CCCCc2ccccc2)CC1] | Phenyl-[4-(4- phenylbutyl)piperazin-1- yl]methanone |
| 25 | 17 | 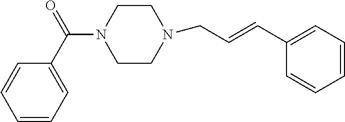 [SMILES: O=C(c1ccccc1)N1CCN(C/C=C/c2ccccc2)CC1] | [4-[(E)-cinnamyl]piperazin- 1-yl]-phenyl-methanone |
| 26 | 18 | 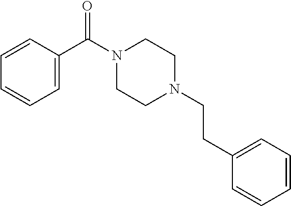 [SMILES: O=C(c1ccccc1)N1CCN(CCc2ccccc2)CC1] | Phenyl-[4-(2- phenylethyl)piperazin-1- yl]methanone |
| 27 | 21 | 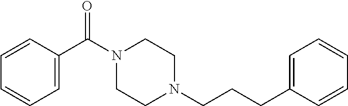 [SMILES: O=C(c1ccccc1)N1CCN(CCCc2ccccc2)CC1] | Phenyl-[4-(3- phenylpropyl)piperazin-1- yl]methanone |
| 28 | 23 | 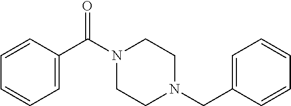 [SMILES: O=C(c1ccccc1)N1CCN(Cc2ccccc2)CC1] | (4-benzylpiperazin-1-yl)- phenyl-methanone |
| 29 | 28 | 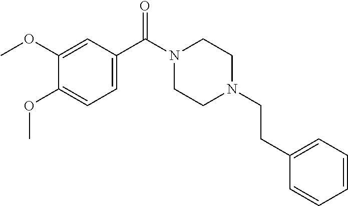 [SMILES: COc1ccc(C(=O)N2CCN(CCc3ccccc3)CC2)cc1OC] | (3,4-Dimethoxyphenyl)-[4- (2-phenylethyl)piperazin-1- yl]methanone |
| 30 | 31 | 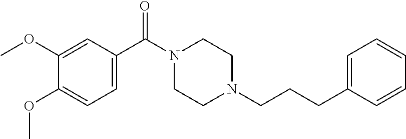 [SMILES: COc1ccc(C(=O)N2CCN(CCCc3ccccc3)CC2)cc1OC] | (3,4-Dimethoxyphenyl)-[4- (3-phenylpropyl)piperazin-1- yl]methanone |
| 31 | 33 | 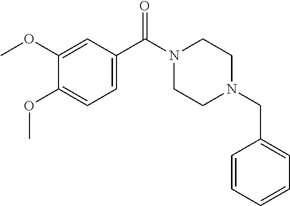 [SMILES: COc1ccc(C(=O)N2CCN(Cc3ccccc3)CC2)cc1OC] | (4-benzylpiperazin-1-yl)- (3,4- dimethoxyphenyl)methanone |
| 32 | 35 | 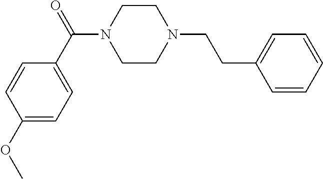 [SMILES: COc1ccc(C(=O)N2CCN(CCc3ccccc3)CC2)cc1] | (4-Methoxyphenyl)-[4-(2- phenylethyl)piperazin-1- yl]methanone |
| 33 | 38 | 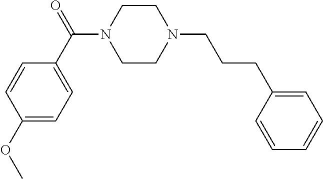 [SMILES: COc1ccc(C(=O)N2CCN(CCCc3ccccc3)CC2)cc1] | (4-Methoxyphenyl)-[4-(3- phenylpropyl)piperazin-1- yl]methanone |
| 34 | 40 | 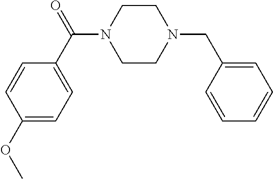 [SMILES: COc1ccc(C(=O)N2CCN(Cc3ccccc3)CC2)cc1] | (4-benzylpiperazin-1-yl)-(4- methoxyphenyl)methanone |
| 35 | 42 | 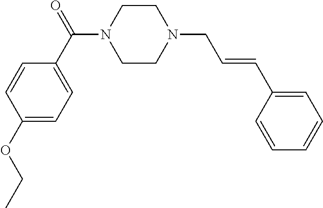 [SMILES: CCOc1ccc(C(=O)N2CCN(C/C=C/c3ccccc3)CC2)cc1] | [4-[(E)-cinnamyl]piperazin- 1-yl]-(4- ethoxyphenyl)methanone |
| 36 | 43 | 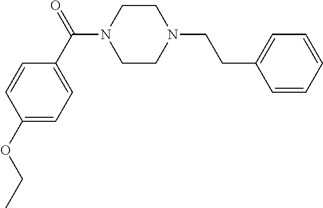 [SMILES: CCOc1ccc(C(=O)N2CCN(CCc3ccccc3)CC2)cc1] | (4-ethoxyphenyl)-[4-(2- phenylethyl)piperazin-1- yl]methanone |
| 37 | 46 | 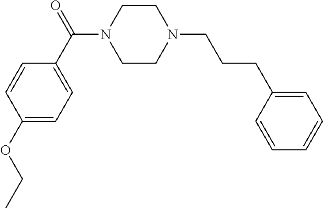 [SMILES: CCOc1ccc(C(=O)N2CCN(CCCc3ccccc3)CC2)cc1] | (4-ethoxyphenyl)-[4-(3- phenylpropyl)piperazin-1- yl]methanone |
| 38 | 48 | 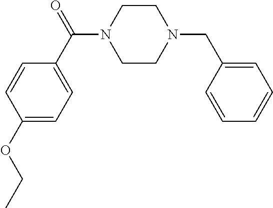 [SMILES: CCOc1ccc(C(=O)N2CCN(Cc3ccccc3)CC2)cc1] | (4-benzylpiperazin-1-yl)-(4- ethoxyphenyl)methanone |

or the active ingredient of the general formula (II) is selected from the group consisting of

| Comp. | | | Chemical |
|---|---|---|---|
| No. | | Compound | Designation |
| 39 | 20 | 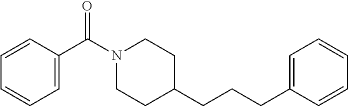 [SMILES: O=C(c1ccccc1)N1CCC(CCCc2ccccc2)CC1] | Phenyl-[4-(3- phenylpropyl)-1- piperidyl]methanone |
| 40 | 25 | 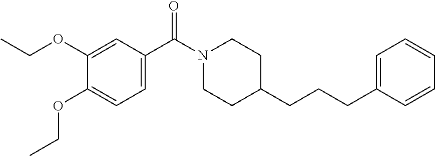 [SMILES: CCOc1ccc(C(=O)N2CCC(CCCc3ccccc3)CC2)cc1OCC] | (3,4-Diethoxyphenyl)- [4-(3-phenylpropyl)-1- piperidyl]methanone |
| 41 | 30 | 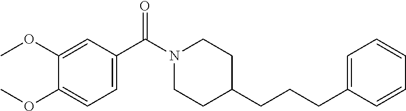 [SMILES: COc1ccc(C(=O)N2CCC(CCCc3ccccc3)CC2)cc1OC] | (3,4- Dimethoxyphenyl)-[4- (3-phenylpropyl)-1- piperidyl]methanone |
| 42 | 37 | 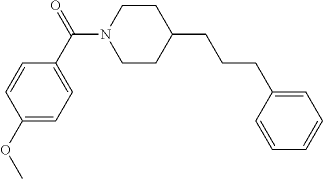 [SMILES: COc1ccc(C(=O)N2CCC(CCCc3ccccc3)CC2)cc1] | (4-Methoxyphenyl)- [4-(3-phenylpropyl)- 1- piperidyl]methanone |
| 43 | 45 | 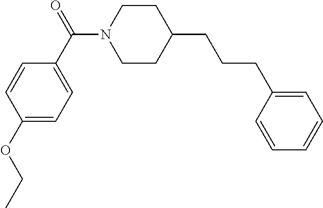 [SMILES: CCOc1ccc(C(=O)N2CCC(CCCc3ccccc3)CC2)cc1] | (4-ethoxyphenyl)-[4- (3-phenylpropyl)-1- piperidyl]methanone |

or the active ingredient of the general formula (III) is selected from the group consisting of

| Comp. | | | Chemical |
|---|---|---|---|
| No. | | Compound | Designation |
| 44 | 22 | 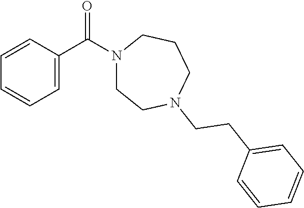 [SMILES: O=C(c1ccccc1)N1CCCN(CCc2ccccc2)CC1] | Phenyl-[4-(2-phenylethyl)- 1,4-diazepan-1- yl]methanone |
| 45 | 26 | 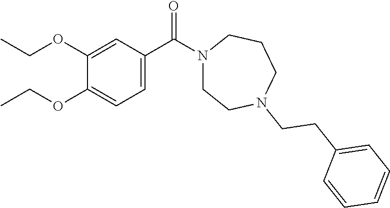 [SMILES: CCOc1ccc(C(=O)N2CCCN(CCc3ccccc3)CC2)cc1OCC] | (3,4-Diethoxyphenyl)-[4-(2- phenylethyl)-1,4-diazepan-1- yl]methanone |
| 46 | 32 | 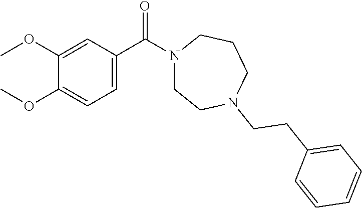 [SMILES: COc1ccc(C(=O)N2CCCN(CCc3ccccc3)CC2)cc1OC] | (3,4-Dimethoxyphenyl)-[4- (2-phenylethyl)-1,4- diazepan-1-yl]methanone |
| 47 | 39 | 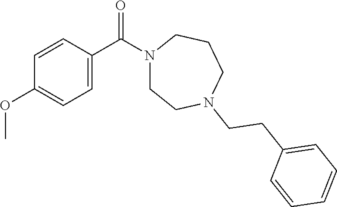 [SMILES: COc1ccc(C(=O)N2CCCN(CCc3ccccc3)CC2)cc1] | (4-Methoxyphenyl)-[4-(2- phenylethyl)-1,4-diazepan-1- yl]methanone |
| 48 | 47 | 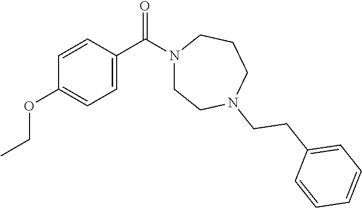 [SMILES: CCOc1ccc(C(=O)N2CCCN(CCc3ccccc3)CC2)cc1] | (4-ethoxyphenyl)-[4-(2- phenylethyl)-1,4-diazepan-1- yl]methanone |

or salts thereof, wherein the active ingredients are present in stereoisomerically pure form or as mixtures of different stereoisomers.

7. The active ingredient according to claim 1, wherein the active ingredient of general formula (I) is selected from the group consisting of:

| Comp. | | | Chemical |
|---|---|---|---|
| No. | | Compound | Designation |
| 1 | 1 | 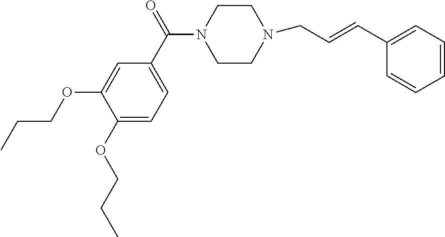 [SMILES: CCCOc1ccc(C(=O)N2CCN(C/C=C/c3ccccc3)CC2)cc1OCCC] | [4-[(E)-cinnamyl]piperazin- 1-yl]-(3,4- dipropoxyphenyl)methanone |
| 2 | 4 | 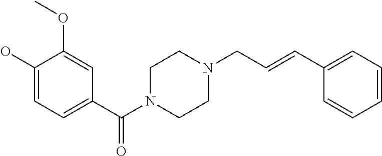 [SMILES: COc1cc(C(=O)N2CCN(C/C=C/c3ccccc3)CC2)ccc1O] | [4-[(E)-cinnamyl]piperazin- 1-yl]-(4-hydroxy-3- methoxy-phenyl)methanone |
| 3 | 6 | 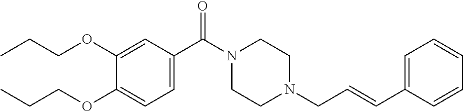 [SMILES: CCCOc1ccc(C(=O)N2CCN(C/C=C/c3ccccc3)CC2)cc1OCCC] | [4-[(E)-cinnamyl]piperazin- 1-yl]-(3,4- dipropoxyphenyl)methanone |
| 4 | 9 | 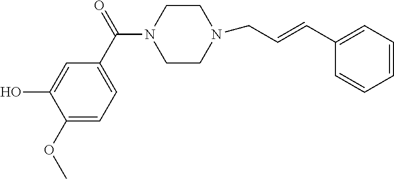 [SMILES: COc1ccc(C(=O)N2CCN(C/C=C/c3ccccc3)CC2)cc1O] | [4-[(E)-cinnamyl]piperazin- 1-yl]-(3-hydroxy-4- methoxy-phenyl)methanone |
| 5 | 10 | 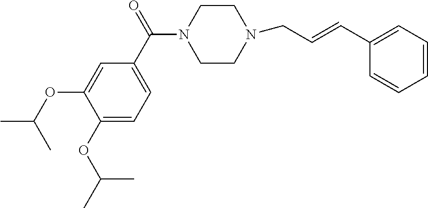 [SMILES: CC(C)Oc1ccc(C(=O)N2CCN(C/C=C/c3ccccc3)CC2)cc1OC(C)C] | [4-[(E)-cinnamyl]piperazin- 1-yl]-(3,4- diisopropoxyphenyl)methanone |
| 6 | 11 | 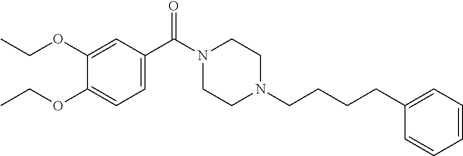 [SMILES: CCOc1ccc(C(=O)N2CCN(CCCCc3ccccc3)CC2)cc1OCC] | (3,4-Diethoxyphenyl)-[4-(4- phenylbutyl)piperazin-1- yl]methanone |
| 7 | 12 | 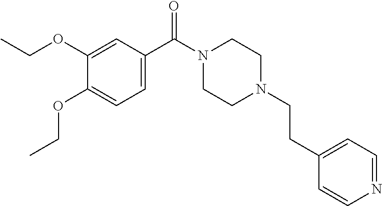 [SMILES: CCOc1ccc(C(=O)N2CCN(CCc3ccncc3)CC2)cc1OCC] | (3,4-Diethoxyphenyl)-[4-[2- (4-pyridyl)ethyl]piperazin-1- yl]methanone |
| 8 | 13 | 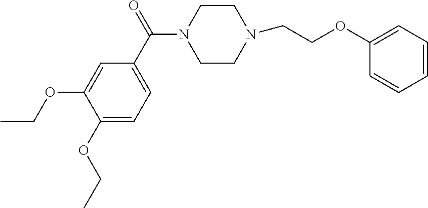 [SMILES: CCOc1ccc(C(=O)N2CCN(CCOc3ccccc3)CC2)cc1OCC] | (3,4-Diethoxyphenyl)-[4-(2- phenoxyethyl)piperazin-1- yl]methanone |
| 9 | 19 | 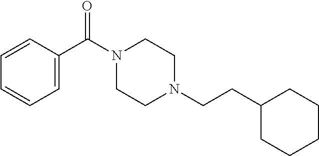 [SMILES: O=C(c1ccccc1)N1CCN(CCC2CCCCC2)CC1] | [4-(2- cyclohexylethyl)piperazin-1- yl]-phenyl-methanone |
| 10 | 24 | 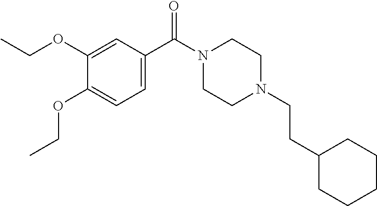 [SMILES: CCOc1ccc(C(=O)N2CCN(CCC3CCCCC3)CC2)cc1OCC] | [4-(2- cyclohexylethyl)piperazin-1- yl]-(3,4- diethoxyphenyl)methanone |
| 11 | 27 | 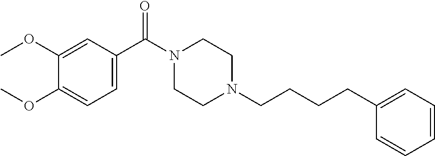 [SMILES: COc1ccc(C(=O)N2CCN(CCCCc3ccccc3)CC2)cc1OC] | (3,4-Dimethoxyphenyl)-[4- (4-phenylbutyl)piperazin-1- yl]methanone |
| 12 | 29 | 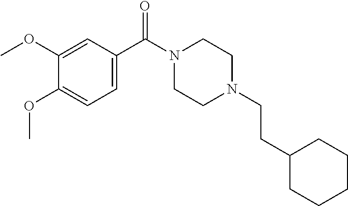 [SMILES: COc1ccc(C(=O)N2CCN(CCC3CCCCC3)CC2)cc1OC] | [4-(2- cyclohexylethyl)piperazin-1- yl]-(3,4- dimethoxyphenyl)methanone |
| 13 | 34 | 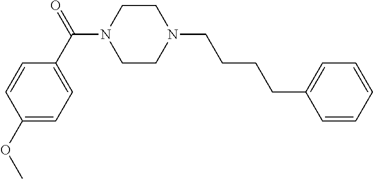 [SMILES: COc1ccc(C(=O)N2CCN(CCCCc3ccccc3)CC2)cc1] | (4-Methoxyphenyl)-[4-(4- phenylbutyl)piperazin-1- yl]methanone |
| 14 | 36 | 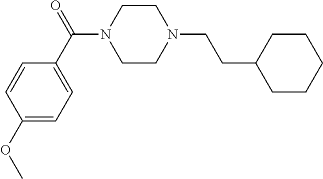 [SMILES: COc1ccc(C(=O)N2CCN(CCC3CCCCC3)CC2)cc1] | [4-(2- cyclohexylethyl)piperazin-1- yl]-(4- methoxyphenyl)methanone |
| 15 | 41 | 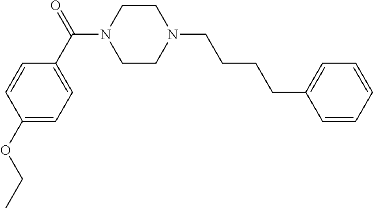 [SMILES: CCOc1ccc(C(=O)N2CCN(CCCCc3ccccc3)CC2)cc1] | (4-ethoxyphenyl)-[4-(4- phenylbutyl)piperazin-1- yl]methanone |
| 16 | 44 | 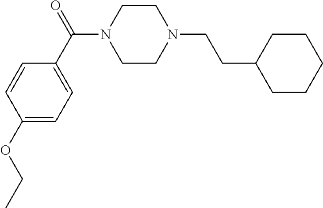 [SMILES: CCOc1ccc(C(=O)N2CCN(CCC3CCCCC3)CC2)cc1] | [4-(2- cyclohexylethyl)piperazin-1- yl]-(4- ethoxyphenyl)methanone |

or the active ingredient of the general formula (II) is selected from the group consisting of

| Comp. | | | Chemical |
|---|---|---|---|
| No. | | Compound | Designation |
| 40 | 25 | 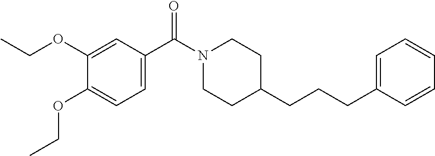 [SMILES: CCOc1ccc(C(=O)N2CCC(CCCc3ccccc3)CC2)cc1OCC] | (3,4-Diethoxyphenyl)-[4-(3- phenylpropyl)-1- piperidyl]methanone |
| 41 | 30 | 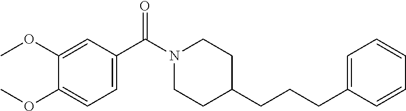 [SMILES: COc1ccc(C(=O)N2CCC(CCCc3ccccc3)CC2)cc1OC] | (3,4-Dimethoxyphenyl)-[4- (3-phenylpropyl)-1- piperidyl]methanone |
| 42 | 37 | 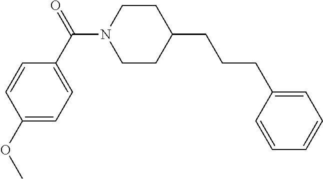 [SMILES: COc1ccc(C(=O)N2CCC(CCCc3ccccc3)CC2)cc1] | (4-Methoxyphenyl)-[4-(3- phenylpropyl)-1- piperidyl]methanone |
| 43 | 45 | 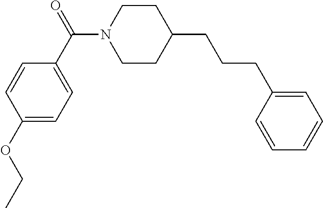 [SMILES: CCOc1ccc(C(=O)N2CCC(CCCc3ccccc3)CC2)cc1] | (4-ethoxyphenyl)-[4-(3- phenylpropyl)-1- piperidyl]methanone |

or the active ingredient of the general formula (III) is selected from the group consisting of

| Comp. | | | Chemical |
|---|---|---|---|
| No. | | Compound | Designation |
| 45 | 26 | 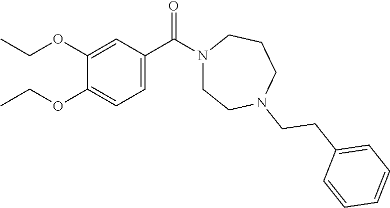 [SMILES: CCOc1ccc(C(=O)N2CCCN(CCc3ccccc3)CC2)cc1OCC] | (3,4-Diethoxyphenyl)-[4-(2- phenylethyl)-1,4-diazepan-1- yl]methanone |
| 46 | 32 | 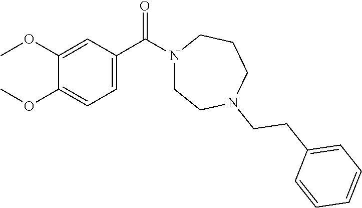 [SMILES: COc1ccc(C(=O)N2CCCN(CCc3ccccc3)CC2)cc1OC] | (3,4-Dimethoxyphenyl)-[4- (2-phenylethyl)-1,4- diazepan-1-yl]methanone |
| 47 | 39 | 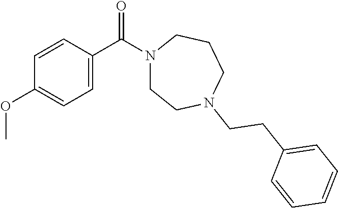 [SMILES: COc1ccc(C(=O)N2CCCN(CCc3ccccc3)CC2)cc1] | (4-Methoxyphenyl)-[4-(2- phenylethyl)-1,4-diazepan-1- yl]methanone |
| 48 | 47 | 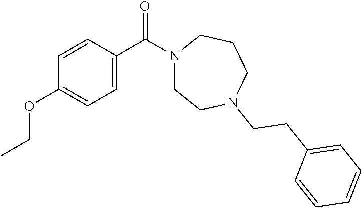 [SMILES: CCOc1ccc(C(=O)N2CCCN(CCc3ccccc3)CC2)cc1] | (4-ethoxyphenyl)-[4-(2- phenylethyl)-1,4-diazepan-1- yl]methanone |

or salts thereof wherein the active ingredients are present in stereoisomerically pure form or as mixtures of different stereoisomers.

8. The active ingredient according to claim 7, wherein the active ingredient of general formula (I) is selected from the group consisting of:

| Comp. | | | Chemical |
|---|---|---|---|
| No. | | Compound | Designation |
| 1 | 1 | 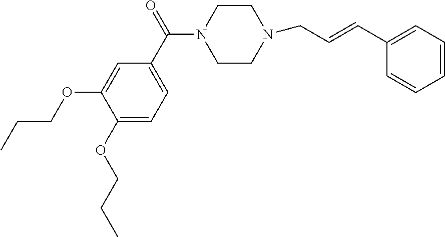 [SMILES: CCCOc1ccc(C(=O)N2CCN(C/C=C/c3ccccc3)CC2)cc1OCCC] | [4-[(E)-cinnamyl]piperazin- 1-yl]-(3,4- dipropoxyphenyl)methanone |
| 6 | 11 | 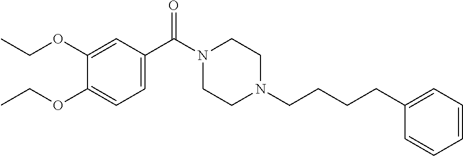 [SMILES: CCOc1ccc(C(=O)N2CCN(CCCCc3ccccc3)CC2)cc1OCC] | (3,4-Diethoxyphenyl)-[4-(4- phenylbutyl)piperazin-1- yl]methanone |
| 7 | 12 | 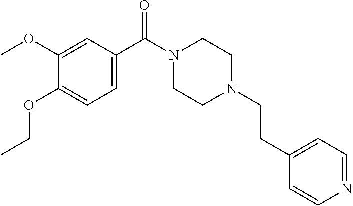 [SMILES: CCOc1ccc(C(=O)N2CCN(CCc3ccncc3)CC2)cc1OC] | (3,4-Diethoxyphenyl)-[4-[2- (4-pyridyl)ethyl]piperazin-1- yl]methanone |
| 8 | 13 | 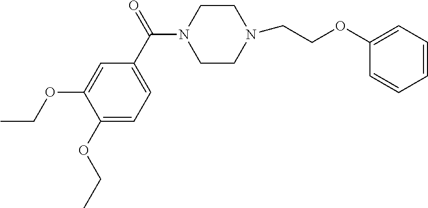 [SMILES: CCOc1ccc(C(=O)N2CCN(CCOc3ccccc3)CC2)cc1OCC] | (3,4-Diethoxyphenyl)-[4-(2- phenoxyethyl)piperazin-1- yl]methanone |
| 9 | 19 | 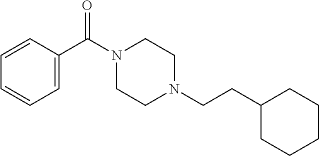 [SMILES: O=C(c1ccccc1)N1CCN(CCC2CCCCC2)CC1] | [4-(2- cyclohexylethyl)piperazin-1- yl]-phenyl-methanone |
| 11 | 27 | 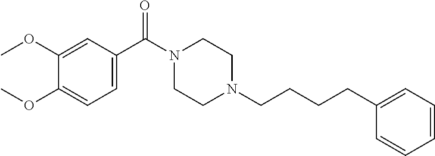 [SMILES: COc1ccc(C(=O)N2CCN(CCCCc3ccccc3)CC2)cc1OC] | (3,4-Dimethoxyphenyl)-[4- (4-phenylbutyl)piperazin-1- yl]methanone |
| 13 | 34 | 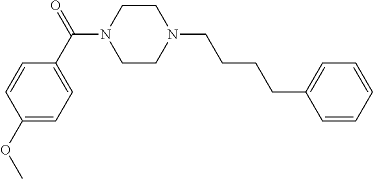 [SMILES: COc1ccc(C(=O)N2CCN(CCCCc3ccccc3)CC2)cc1] | (4-Methoxyphenyl)-[4-(4- phenylbutyl)piperazin-1- yl]methanone |
| 14 | 36 | 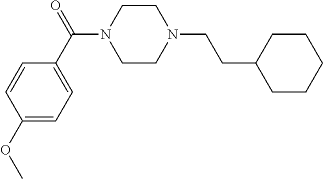 [SMILES: COc1ccc(C(=O)N2CCN(CCC3CCCCC3)CC2)cc1] | [4-(2- cyclohexylethyl)piperazin-1- yl]-(4- methoxyphenyl)methanone |
| 15 | 41 | 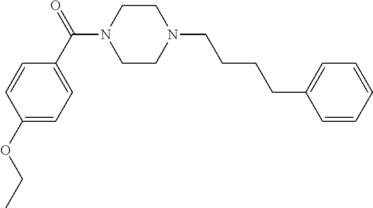 [SMILES: CCOc1ccc(C(=O)N2CCN(CCCCc3ccccc3)CC2)cc1] | (4-ethoxyphenyl)-[4-(4- phenylbutyl)piperazin-1- yl]methanone |
| 16 | 44 | 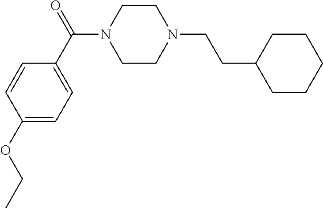 [SMILES: CCOc1ccc(C(=O)N2CCN(CCC3CCCCC3)CC2)cc1] | [4-(2- cyclohexylethyl)piperazin-1- yl]-(4- ethoxyphenyl)methanone |

or the active ingredient of the general formula (II) is selected from the group consisting of

| Comp. | | | Chemical |
|---|---|---|---|
| No. | | Compound | Designation |
| 43 | 45 | 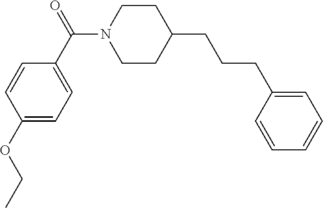 [SMILES: CCOc1ccc(C(=O)N2CCC(CCCc3ccccc3)CC2)cc1] | (4-ethoxyphenyl)-[4-(3- phenylpropyl)-1- piperidyl]methanone |

or the active ingredient of the general formula (III) is selected from the group consisting of

| Comp. | | | Chemical |
|---|---|---|---|
| No. | | Compound | Designation |
| 47 | 39 | 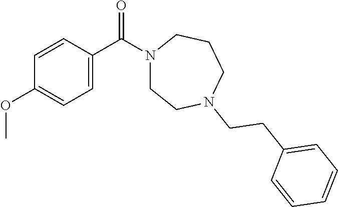 [SMILES: COc1ccc(C(=O)N2CCCN(CCc3ccccc3)CC2)cc1] | (4-Methoxyphenyl)-[4-(2- phenylethyl)-1,4-diazepan- 1-yl]methanone |
| 48 | 47 | 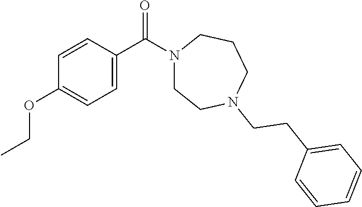 [SMILES: CCOc1ccc(C(=O)N2CCCN(CCc3ccccc3)CC2)cc1] | (4-ethoxyphenyl)-[4-(2- phenylethyl)-1,4-diazepan- 1-yl]methanone |

or salts thereof, whereby the active ingredients are present in stereoisomerically pure form or as mixtures of different stereoisomers.

9. A flavoring preparation comprising
(a) one or more active ingredient(s) according to claim 1:
or salts thereof, whereby the active ingredients are present in stereoisomerically pure form or as mixtures of different stereoisomers.

10. The flavoring preparation according to claim 9, further comprising:
(b) at least one further active ingredient which is different from the active ingredient (a) of the general formulae (I) to (III).

11. The flavoring preparation according to claim 10, wherein the further active ingredient other than the active ingredient (a) of general formulae (I) to (III) is selected from the group consisting of:
Heat-inducing or pungent active ingredients selected from the group consisting of: Capsaicinoids; gingerols; shogaols; gingerdiones; paradols; dehydrogingerdiones; piperine; piperine derivatives; ethyl 2-(4-hydroxy-3-methoxy-phenyl)acetate and 3-phenylpropyl-2-(4-hydroxy-3-methoxy-phenyl)acetate and mixtures thereof;
active ingredients which can be perceived as pungent or acrid, selected from the group consisting of: aromatic isothiocyanates;
Alkamide active ingredients described as tingling, selected from the group consisting of 2E,4E-decadienoic acid-N-isobutylamide (trans-pellitorin); 2E,4Z-decadienoic acid-N-isobutylamide (cis-pellitorin); 2Z,4Z-decadienoic acid-N-isobutylamide; 2Z,4E-decadienoic acid-N-isobutylamide; 2E,4E-decadienoic acid-N-([2S]-2-methylbutyl)amide; 2E,4E-decadienoic acid-N-([2S]-2-methylbutyl)amide; 2E,4E-decadienoic acid-N-([2R]-2-methylbutylamide); 2E,4Z-decadienoic acid-N-(2-methylbutyl)amide; 2E,4E-decadienoic acid-N-piperide (achilleamide); 2E,4E-decadienoic acid-N-piperide (sarmentin); 2E-decenoic acid-N-isobutylamide; 3E-decenoic acid-N-isobutylamide; 3E-nonenoic acid-N-isobutylamide; 2E,6Z,8E-decatrienoic acid-N-isobutylamide (spilanthol); 2E,6Z,8E-decatrienoic acid-N-([2S]-2-methylbutyl)amide (homospilanthol); 2E,6Z,8E-decatrienoic acid-N-([2R]-2-methylbutyl)amide; 2E-decen-4-ynoic acid-N-isobutylamide; 2Z-decen-4-ynoic acid-N-isobutylamide; 2E,6Z,8E,10E-dodecatetraenoic acid-N-(2-methylpropyl)amide (alpha-sanshool); 2E,6Z,8E,10E-dodecatetraenoic acid-N-(2-hydroxy-2-methylpropyl)amide (alpha-hydroxysanshool); 2E,6E,8E,10E-dodecatetraenoic acid-N-(2-hydroxy-2-methylpropyl)amide (gamma-hydroxysanshool); 2E,4E,8Z,10E,12E-tetradecapentaenoic acid-N-(2-hydroxy-2-methylpropyl)amide (gamma-hydroxysanshool); 2E,4E,8E,10E,12E-tetradecapentaenoic acid-N-(2-hydroxy-2-methylpropyl)-amide (gamma-hydroxyisosanshool); 2E,4E,8Z,10E,12E-tetradecapentaenoic acid-N-(2-methyl-2-propenyl)amide (gamma-dehydrosanshool); 2E,4E,8Z,10E,12E-tetradecapentaenoic acid-N-(2-methylpropyl)amide (gamma-sanshool); 2E,4E,8Z,11Z-tetradecatetraenoic acid-N-(2-hydroxy-2-methylpropyl)amide (bungeanool); 2E,4E,8Z,11E-tetradecatetraenoic acid-N-(2-hydroxy-2-methylpropyl)amide (isobungeanool); 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)amide (tetrahydrobungeanool) and mixtures thereof;
Active ingredients with astringent effect, selected from the group consisting of: Catechins; Dihydroflavonoids, flavonols, rutin, gallic acid esters of carbohydrates, pentagalloyl glucose or their reaction products, aluminum salts, as well as their mixtures; and
Active ingredients with physiological cooling effect, selected from the group consisting of: Menthol, menthol methyl ether (FEMA GRAS 4054), monomenthyl glutamate (FEMA GRAS 4006), menthoxy-1,2-propanediol (FEMA GRAS 3784), dimenthyl glutarate (FEMA GRAS 4604), Hydroxymethylcyclohexylethanone (FEMA GRAS 4742), 2-(4-ethylphenoxy)-N-(1H-pyrazol-3-yl)-N-(thiophen-2-ylmethyl)acetamide (FEMA GRAS 4880), WS-23 (2-isopropyl-N,2,3-trimethylbutyramide (FEMA GRAS 3804), N-(4-(cyanomethyl)phenyl)-2-isopropyl-5,5-dimethylcyclohexane carboxamide (FEMA GRAS 4882), N-(3-hydroxy-4-methoxyphenyl)-2-isopropyl-5,5-dimethylcyclohexane carboxamide (FEMA GRAS 4881), N-(2-hydroxy-2-phenylethyl)-2-isopropyl-5,5-dimethylcyclohexane-1-carboxamide (FEMA GRAS 4896), 3,4-methylenedioxy cinnamic acid, (E)-3-benzo[1,3]dioxol-5-yl-N,N-diphenyl-2-propenamide (FEMA GRAS 4788), menthol propylene glycol carbonate (FEMA GRAS 3806), menthyl N-ethyloxamate, monomethyl succinate (FEMA GRAS 3810), WS-3 (N-ethyl-p-menthane-3-carboxamide, FEMA GRAS 3455), menthol ethylene glycol carbonate (FEMA GRAS 3805), WS-5 (ethyl 3-(p-menthane-3-carboxamido)acetate, FEMA GRAS 4309), WS-12 (1R,2S,5R)—N-(4-methoxyphenyl)p-menthanecarboxamide (FEMA GRAS 4681), WS-27 (N-ethyl-2,2-diisopropylbutanamide, FEMA GRAS 4557), N-cyclopropyl-5-methyl-2-isopropylcyclohexanecarboxamide (FEMA GRAS 4693), WS-116 (N-(1,1-dimethyl-2-hydroxyethyl)-2,2-diethylbutanamide, FEMA GRAS 4603), menthoxyethanol (FEMA GRAS 4154), N-(4-cyanomethylphenyl)-p-menthanecarboxamide (FEMA GRAS 4496), N-(2-(pyridin-2-yl)ethyl)-3-p-menthanecarboxamide (FEMA GRAS 4549), N-(2-hydroxyethyl)-2-isopropy-1-2,3-dimethylbutanamide (FEMA GRAS 4602), (2S,5R)—N-[4-(2-amino-2-oxoethyl)phenyl]-p-menthanecarboxamide (FEMA GRAS 4684), N-cyclopropyl-5-methyl-2-isopropylcyclohexanecarboxamide (FEMA GRAS 4693), 2-[(2-p-menthoxy)ethoxy]-ethanol (FEMA GRAS 4718), (2,6-diethyl-5-isopropyl-2-methyltetrahydropyran (FEMA GRAS 4680), trans-4-tert-butylcyclohexanol (FEMA GRAS 4724), 2-(p-Tolyloxy)-N-(1H-pyrazol-5-yl)-N((thiophen-2-yl)methyl)acetamide (FEMA GRAS 4809), menthone glycerol ketal (FEMA GRAS 3807 and 3808), (−)-menthoxypropane-1,2-diol, 3-(1-menthoxy)-2-methylpropane-1,2-diol (FEMA GRAS 3849), isopulegol, (+)-cis and (−)-trans-p-menthane-3,8-diol (62:38, FEMA GRAS 4053), 2,3-dihydroxy-p-menthane, 3,3,5-trimethylcyclohexanone glycerol ketal, menthyl pyrrolidone carboxylate, (1R,3R,4S)-3-menthyl-3,6-dioxaheptanoate, (1R,2S,5R)-3-menthyl methoxyacetate, (1R,2S,5R)-3-menthyl-3,6,9-trioxadecanoate, (1R,2S,5R)-3-menthyl-3,6,9-trioxadecanoate, (1R,2S,5R)-3-menthyl-(2-hydroxyethoxy)acetate, (1R,2S,5R)-menthyl-11-hydroxy-3,6,9-trioxaundecanoate, cubebol (FEMA GRAS 4497), 2-isopropyl-5-methylcyclohexyl-4-(dimethylamino)-4-oxobutanoate (FEMA GRAS 4230), menthyl lactate (FEMA GRAS 3748), 6-isopropyl-3,9-dimethyl-1,4-dioxaspiro[4.5]decan-2-one (FEMA GRAS 4285), N-benzo[1,3]-dioxol-5-yl-3-p-menthanecarboxamide, N-(1-isopropyl-1,2-dimethylpropyl)-1,3-benzodioxole-5-carboxamide, N—(R)-2-oxotetrahydrofuran-3-yl-(1R,2S,5R)-p-menthane-3-carboxamide, mixture of 2,2,5,6,6-pentamethyl-2,3,6,6a-tetrahydropentalene-3a(1H)-ol and 5-(2-hydroxy-2-methylpropyl)-3,4,4-trimethylcyclopent-2-en-1-one; (2S,5R)-2-isopropyl-5-methyl-N-(2-(pyridin-4-yl)ethyl)cyclohexanecarboxamides; (1S,2S,5R)—N-(4-(cyanomethyl)phenyl)-2-isopropyl-5-methylcyclohexanecarboxamide, 1,7-isopropyl-4,5-methyl-bicyclo[2.2.2]oct-5-ene derivatives, 4-methoxy-N-phenyl-N-[2-(pyridin-2-yl)ethyl]benzamide, 4-methoxy-N-phenyl-N-[2-(pyridin-2-yl)ethyl]benzenesulfonamide, 4-chloro-N-phenyl-N-[2-(pyridin-2-yl)ethyl]benzenesulfonamide, 4-cyano-N-phenyl-N-[2-(pyridin-2-yl)ethyl]-benzenesulfonamide, 4-((benzhydrylamino)methyl)-2-methoxyphenol, 4-((bis(4-methoxyphenyl)methylamino)methyl)-2-methoxyphenol, 4-((1,2-diphenylethylamino)methyl)-2-methoxyphenol, 4-((benzhydryloxy)methyl)-2-methoxyphenol, 4-((9H-fluoren-9-ylamino)methyl)-2-methoxyphenol, 4-((benzhydrylamino)methyl)-2-ethoxyphenol, 1-(4-methoxyphenyl)-2-(1-methyl-1H-benzo[d]imidazol-2-yl)vinyl-4-methoxybenzoate, 2-(1-isopropyl-6-methyl-1H-benzo[d]imidazol-2-yl)-1-(4-methoxyphenyl)vinyl-4-methoxybenzoate, (Z)-2-(1-isopropyl-5-methyl-1H-benzo[d]imidazol-2-yl)-1-(4-methoxyphenyl)vinyl-4-methoxybenzoate, 3-alkyl-p-methan-3-ol derivatives, derivatives of fenchyl, D-bornyl, L-bornyl, exo-norbornyl, 2-methylisobornyl, 2-ethylfenchyl, 2-methylbornyl, cis-pinan-2-yl, verbanyl and isobornyl, menthyloxamate derivatives, menthyl 3-oxocarboxylic acid esters, N-alpha-(menthanecarbonyl)amino acid amides, p-menthane carboxamide and WS-23 analogs, (−)-(1R,2R,4S)-dihydroumbellulol, p-menthane alkyloxyamide, cyclohexane derivatives, butanone derivatives, mixture of 3-menthoxy-1-propanol and 1-menthoxy-2-propanol, 1-[2-ydroxyphenyl]-4-[2-nitrophenyl]-1,2,3,6-tetrahydropyrimidin-2-one, 4-methyl-3-(1-pyrrolidinyl)-2-[5H]-furanone and mixtures thereof.

12. The flavor preparation according to claim 9 comprising component (a) in an amount of 0.1 to 20% by weight, based on the total weight of the flavor preparation.

13. The active ingredient according to claim 1 or a flavoring preparation comprising one or more active ingredient(s) according claim 1 in encapsulated form.

14. A method of stimulating salivation in the mouth, producing a tingling/fizzing sensation in the mouth, and/or enhancing a pleasant taste sensation in the mouth comprising formulating a flavoring agent with an active ingredient according to claim 1.

15. A formulation comprising an active ingredient according to claim 1 or a flavoring preparation comprising one or more active ingredient(s) according to claim 1, wherein the formulation is foodstuffs, food supplements, luxury foods, cosmetic or pharmaceutical preparations, tobacco products or animal feed.

16. The formulation of claim 15 wherein the active ingredient is in an amount of 0.001 ppm to 1000 ppm, based on the total weight of the final formulation.

17. A method of preventing and/or treating dry mouth or xerostomia comprising administering a pharmaceutical preparation according to claim 16 to a subject in need thereof.

18. The flavor preparation according to claim 10 comprising component (a) plus component (b) in an amount of 0.1 to 20% by weight based on the total weight of the flavor preparation, and/or wherein component (a) and component (b) are contained in a weight ratio of about 0.1:99.9 to about 99.9:0.1.
